(19) United States
(12) Patent Application Publication
(10) Pub. No.: US 2009/0178158 A1
(43) Pub. Date: Jul. 9, 2009

(54) RESISTANCE AGAINST PARASITIC WEEDS

(75) Inventors: Hendrik Jan Bouwmeester, Renkum (NL); Radoslava Matusova, Nitra (SK); Zhongkui Sun, Tianjin (CN); Michael Beale, Hertfordshire (GB); Kumkum Rani, Meerut (IN)

Correspondence Address: FOLEY AND LARDNER LLP;SUITE 500, 3000 K STREET NW, WASHINGTON, DC, 20007, US

(73) Assignees: Plant Research International B.V.; Rothamsted Research Limited

(21) Appl. No.: 11/908,904

(22) PCT Filed: Mar. 20, 2006
(86) PCT No.: PCT/NL2006/050059

§ 371 Date: Jan. 29, 2008

(30) Foreign Application Priority Data
Mar. 18, 2005 (EP) 05102164.0

(51) Int. Cl. C12N 15/82; A01H 5/00; C12Q 1/68; A01N 43/40; A01N 43/58; A01P 13/02

(52) U.S. Cl. 800/279; 800/301; 435/6; 504/254; 504/237

(57) ABSTRACT

The seeds of parasitic plants of the genera Striga and Orobanche will only germinate after induction by a chemical signal exuded from the roots of their host. Many of these compounds have been isolated and identified from a number of different plant species and are collectively called the strigolactones. Here we show that the strigolactone germination stimulants are derived from the carotenoid pathway. This finding is used to create crop species that do not induce germination of parasitic plant seeds anymore and therefore are resistant to parasitic plants. Also provided is a method to use chemicals and mycorrhizae to inhibit germination stimulant production to control parasitic plants. Also provided are strigolactone overproducing trap and catch crops.

### FIELD OF THE INVENTION

The present invention relates to the field of plant biotechnology and plant breeding. In particular methods for making plants having enhanced resistance to parasitic weeds are provided. Further provided is a method to use specific herbicides and/or mycorrhiza to control parasitic plants through their effect on the host plant. Also provided are methods for making trap plants and catch plants for parasitic weed control, as well as chimeric genes, overexpression vectors and gene silencing vectors for use in any of these methods. Also, recombinant plants and plant cells, tissues and organs are provided.

### BACKGROUND OF THE INVENTION

Parasitic weeds cause enormous yield losses in agriculture. Broomrapes (Orobanche spp., Orobanchaceae) and witchweeds (Striga spp., Scrophulariaceae) are serious pests in many countries. Infected crops can be heavily damaged even before Orobanche; or Striga emerge above the soil. Orobanche spp. are holoparasites that lack chlorophyll and for their development they obtain water and nutrients through the roots of their host. Orobanche cumana Wallr. parasitises sunflower in Spain as well as in Eastern Europe around the Black Sea (Akhtouch et al., 2002, Plant Breeding, 121, 266-268) Orobanche cernua is closely related to O. cumana but parasites a wider range of hosts, mainly Solanaceous species. Orobanche ramosa is widely spread in Southern Europe and the Mediterranean region and was introduced to regions of South Africa, USA and Central America (Musselman, 1994, Biology and management of Orobanche. Procceedings of the Third International Workshop on Orobanche and related Striga research, 27-35). Together with Orobanche aegyptiaca from which it is difficult to distinguish, O. ramosa parasitises a wide range of hosts such as tomato, potato, eggplant, tobacco, cucurbits, crucifers, sunflower and some other vegetables such as carrot, celery, parsnip and lettuce (Press et al., 2001, The World's worst weeds, 71-90) Orobanche crenata is a widespread parasite of legumes all around the Mediterranean (Press et al., 2001, supra). Striga spp. belong to the hemiparasites with lower photosynthetic activity and basically behave as holoparasites (Parker and Riches, 1993, 111-163). Striga spp. are a problem particularly in the African continent, but also extend into Asia (Press et al., 2001, supra). Hosts of the most important agriculturally important Striga spp, S. hermonthica, Striga asiatica, Striga aspera, Striga forbesii include grain cereals such as maize, sorghum, millet and upland rice (Press et al., 2001, supra). Striga gesneroides is a parasite of cowpea, and causes extensive damage in dry areas of Sub-Saharan, particularly West-Africa (Press et al., 2001, supra).

The first critical step in the life cycle of these parasites—germination of their seeds, is regulated by specific chemical signals exuded by the roots of host plants. For Striga spp. several germination stimulants were identified from host and non-host plants. Most of them are known as strigolactones (FIG. 1). Germination stimulants in maize and sorghum were identified as strigol (Siame et al., 1993, J Agr Food Chem, 41, 1486-1491) and sorgolactone (Hauck et al., 1992, J Plant Physiol, 139, 474-478). Alectrol was identified in the root exudate of cowpea (Muller et al., 1992, J Plant Growth Regul, 11, 77-84). Alectrol and orobanchol, germination stimulants for O. minor were isolated and identified from the root exudate of red clover (Yokota et al., 1998, Phytochemistry, 49, 1967-1973). The same group recently reported on the isolation of four novel strigolactones from the root exudate of tomato, and the presence of a novel strigol-isomer in the root exudate of sorghum (Yoneyama et al., 2004, 8th International Parasitic Weed Symposium, 9).

Although Striga and Orobanche spp parasitise different hosts in different parts of the world, their lifecycles are principally similar, and hence we will discuss the two genera together. The important steps in the lifecycle are germination, radicle growth to the host root, haustorium formation and attachment to the host root, the establishment of a xylem connection and compatible interaction, and seed production. In many of the steps there is extensive signalling between the host plant and the parasite. This begins with the secretion of secondary metabolites from the roots of hosts [and some false (non) hosts] that induce the germination of the parasite seeds. For the seeds of Orobanche and Striga spp. to become responsive to these germination stimulants they require a moist environment for a certain period of time at a suitable temperature. This period is described as preconditioning or conditioning and is comparable to what is called (warm) stratification in seeds of non-parasitic plants or release of dormancy (Matusova et al., 2004, Seed Sci Res, 14, 335-344). During preconditioning of parasitic plant seeds the dormancy of the seeds is broken and they become progressively more responsive to germination stimulants. After reaching the maximum sensitivity, the seeds start entering into secondary dormancy and their sensitivity to the germination stimulants gradually decreases. Hence, the length of the preconditioning period has a great effect on the sensitivity of the parasitic weed seeds to germination stimulants, similar to the sensitivity of non-parasitic plant seeds to other external stimuli such as light and nitrate (Matusova et al., 2004, supra).

The adaptation of the parasitic weeds to these germination stimulants is of evolutionary significance as the tiny seeds contain minimal reserves and thus cannot survive for more than a few days after germination unless a host root is invaded (Butler, 1995, Allelopathy: organism, processes and application, 158-168). After germination, further host-derived secondary metabolites are involved in the plant-parasite interaction. For example, sunflower-derived allelochemical coumarins induce necrosis in the germinated seeds of O. cumana (Serghini et al., 2001, J Exp Bot, 52, 2227-2234). Also, the radicle of the parasite must grow towards the host root and this process may be directed by host-root-derived compounds, perhaps by the concentration gradient of germination stimulant (Dube and Olivier, 2001, Can J Bot, 79, 1225-1240). On encountering a host root, attachment to the root and the host xylem vessels is realised by formation of a haustorium, which is also initiated and guided by host-derived secondary metabolites (Hirsch et al., 2003, Ecology, 84, 858-868; Keyes et al., 2001, Plant Physiol., 127, 1508-1512; Yoder, 2001, Curr Opin Plant Biol, 4, 359-365). Finally, after haustorium formation a connection to the host root xylem is established, probably with involvement of hydrolytic enzymes produced by the penetrating parasite (Labrousse et al., 2001, Ann Bot-London, 88, 859-868). Several authors have shown that the success of this process, i.e. the establishment of a xylem connection, is also dependent on the host and can be negated by host produced toxins (Goldwasser et al., 1999, Physiol Mol Plant P, 54, 3-4; Labrousse et al., 2001, supra; Serghini et al., 2001, supra).

### Germination Stimulants

As mentioned above, the strigolactones have been identified in the root exudates of a variety of plant species (FIG. 1). Strigol was first identified in the false host cotton (Cook et al., 1972, J Am Chem Soc, 94, 6198-6199) and later also in the true Striga hosts maize, sorghum and millet (Butler, 1995, supra; Hauck et al., 1992, supra; Siame et al., 1993, supra). The structurally related alectrol was identified in cowpea, a host of S. gesneroides (Muller et al., 1992, supra). The first Orobanche germination stimulants alectrol, orobanchol and a third unidentified germination stimulant have been isolated from root exudate of red clover (Yokota et al., 1998, supra). The same group recently reported on the isolation of four novel strigolactones from the root exudate of tomato, and the presence of a novel strigol-isomer in the root exudate of sorghum (Yoneyama et al., 2004, supra).

The chemical structures of the four strigolactones identified so far are small variations on one molecular backbone (FIG. 1) and it is tempting to speculate that the small variations in structure of these compounds play a role in the host specificity of the parasitic weeds. Recognition of germination stimulants may ensure that seeds of parasites only germinate in the presence of a true host. However, a number of examples show that the specificity may not be very high. Wigchert et al. (Wigchert and Zwanenburg, 1999, J Agr Food Chem, 47, 1320-1325) induced germination of the seeds of O. crenata—that normally parasitises legumes—with sorgolactone, one of the germination stimulants identified in sorghum. Alectrol was identified in cowpea as a germination stimulant for S. gesneroides (Muller et al., 1992, supra), but was also identified in red clover as a germination stimulant for O. minor (Yokota et al., 1998, supra). Finally, the synthetic strigolactone analogue GR 24 (FIG. 1) induces germination of many parasitic weed seeds regardless of parasite or host plant species. On the other hand, there is some degree of host specificity. For example, not all host plant species induce germination of all parasitic weed seeds and not all synthetic germination stimulants induce germination of all parasites to the same extent (Mwakaboko, 2003). Furthermore, different races amongst parasitic weed populations appear to have evolved to recognise germination stimulants from crops parasitised by the parent weed (Gurney et al., 2002, Weed Res, 42, 299-306).

Although there is not a single determinant in a successful interaction between parasite and host plant, the production of a germination stimulant is a prerequisite. Other factors are the presence of ‘conditioned’ parasite seed (determined by suitable environmental conditions such as temperature and moisture) and compatibility between parasite and host. The first step in the interaction between host and parasite—induction of germination—is an important target for improved control measures. In sorghum a selection program for low-germination stimulant formation has resulted in low-stimulant sorghum varieties with improved resistance (or decreased sensitivity) (Mohamed et al., 2001, 7th International Parasitic Weed Symposium, 96-100). Work on synthetic germination stimulants in the group of B Zwanenburg has led to the development of molecules that have potential as parasitic weed control agents through the induction of suicidal germination (Mwakaboko, 2003, supra; Wigchert and Zwanenburg, 1999, supra). Another control strategy, based on the production of germination stimulants by non-hosts, is the use of trap and catch crops in monoculture or in intercropping. Usually, these non-host crops produce germination stimulants, sometimes in high amounts, and hence induce massive germination of the parasite, but they are resistant in a later stage of the parasite's lifecycle (trap crops) or harvested before the seeds of the parasite are shed (catch crops) (Chittapur et al., 2001, Allelopathy J, 8, 147-160).

### Biosynthetic Origin of Germination Stimulants

Surprisingly, little is known about the biochemical pathway and regulation of the biosynthesis of germination stimulants in the roots of the host species. This is without doubt due to the extremely low concentrations of highly active compounds that are produced by and secreted from the host roots. The strigolactones have been described as sesquiterpene lactones by many authors. Thus, control methods have so far focused on modulating the sequiterpene pathway, for example using chemical inhibitors. The present inventors surprisingly found that germination stimulants are synthesized via the carotenoid pathway, which allows for the first time to devise methods for reducing or increasing the production of germination stimulants.

Therefore, in one embodiment, the present invention provides methods of making low-stimulant producing plants or plant varieties, which have enhanced resistance to one or more species of parasitic weeds. In addition, the present invention shows that sub-lethal concentrations of carotenoid biosynthesis inhibitors can reduce the formation of germination stimulants. Therefore, in another embodiment, the present invention provides a method to use herbicides (carotenoid biosynthesis inhibitors, such as fluridone, norflurazone, isoxaflutole, flurtamone, clomazone, fluorochloridone, pyridazinone, nicotinanilide, amitrol, naproxen and/or abamine, or others) to reduce parasitic weed infestation.

The parasitic weed resistance mechanisms of the invention (modulating levels of germination stimulants) may be combined with other resistance mechanisms, such as incompatibility or the presence of phytoalexins, for more durable resistance. Also provided are high stimulant producing plants or varieties that are more efficient trap or catch crops and/or can induce suicidal germination at greater distances from the plant root.

Most agricultural plants form arbuscular mycorrhizas, a beneficial relationship between plant roots and certain root-inhabiting fungi. Interestingly, two groups have reported that mycorrhiza can reduce Striga infection of sorghum and maize (Gworgwor and Weber, 2003, Mycorrhiza, 13, 277-281; Lendzemo, 2004, PhD thesis, ; Lendzemo and Kuyper, 2001, Agr Ecosyst Environ, 87, 29-35). The mechanism of this reduction was so far unknown, and therefore the possibilities to optimise and explore this phenomenon were limited. In the present invention, we show that this reduction is due to a decrease in germination stimulant formation after mycorrhizal colonisation. Therefore, in one embodiment, the present invention provides a method to optimise the use of mycorrhizae for controlling parasitic plants by using a germination bioassay to analyse the effect of mycorrhizae on germination stimulant formation.

### GENERAL DEFINITIONS

“Germination stimulants” or “parasitic weed seed germination stimulants” refer to strigolactones, which are capable of stimulating the seed germination of parasitic weed species, especially Striga and Orobanche species, but are also known to be the “branching factor” that mycorrhizae need to recognise and colonise their host (Akiyama et al., 2005, Nature 435, 824-827).

The strigolactones have the chemical formula as depicted in FIG. 1. Individual members of the strigolactones are strigol, sorgholactone, alectrol and orobanchol. The (tentative) identification of other strigolactones such as 1 dehydro- and 3 tetradehydro-strigol isomers and unknown strigolactones in the root exudates of sorghum and red clover demonstrate that probably (many) more members of this class exist that have not been discovered yet, and which have similar activity as the already described members.

“Isoprenoids” are molecules having a carbon skeleton derived from isoprene [CH2=C(CH3)CH═CH2], and are subdivided into groups based on their carbon number, e.g. C10 monoterpenes, C15 sesquiterpenes, C20 diterpenes, C25 sesterterpenes, C30 triterpenes, C40 tetraterpenes and C5n polyterpenes.

“Carotenoids” are C40 isoprenoids, derived from eight isoprene units whose order is inverted at the molecular center (FIG. 2). Carotenoids are classified by their chemical structure, with carotenes being hydrocarbons and oxycarotenoids or xanthophylls having additional oxygen. In addition, carotenoids can be classified as primary or secondary, where primary carotenoids are required in photosynthesis (β-carotene, violaxanthin and neoxanthin) whereas secondary carotenoids are localised in fruits and flowers (Delgado-Vargas et al., 2000, Crit. Rev Food Sci, 40, 173-289). “Apocarotenoids” are carotenoid degradation products, such as for example the fragrance volatiles α and β-ionone, the pigment bixin (annatto), the mycorrhiza-induced compounds mycorradicin and blumenin and the plant hormone abscisic acid.

“Carotenoid pathway” is the biosynthetic pathway of the carotenoids. This pathway starts from the condensation of two molecules of geranylgeranyl diphosphate to form the first C40 carotenoid molecule phytoene and ends at any enzymatic or non-enzymatic carotenoid cleavage step which leads to the formation of apocarotenoids (see above).

Whenever “Striga” is used we mean one or more of the Striga spp. Whenever “Orobanche” is used we mean one or more of the Orobanche spp. “Parasitic plants” or “parasitic weeds” refers thus to plant species of the genus Striga and/or Orobanche. The term “nucleic acid sequence” (or nucleic acid molecule) refers to a DNA or RNA molecule in single or double stranded form, particularly a DNA encoding a protein or protein fragment according to the invention. An “isolated nucleic acid sequence” refers to a nucleic acid sequence which is no longer in the natural environment from which it was isolated, e.g. the nucleic acid sequence in a bacterial host cell or in the plant nuclear or plastid genome.

“Target enzyme(s)” or “carotenoid pathway enzymes” are enzymes involved in carotenoid biosynthesis or catabolism (e.g. carotenoid catabolism to strigolactone germination stimulants) and wherein the genes encoding these enzymes are suitable targets for changing (e.g. enhancing or reducing) strigolactone germination stimulant formation in a plant cell, tissue or plant, e.g. by modulating their expression using e.g. gene silencing or overexpression approaches. Herein two categories of target enzymes are referred to: (i) “primary carotenoid pathway enzymes”, which are enzymes from the primary carotenoid/ABA biosynthetic pathway (see also FIG. 2), such as, but not limited to, phytoene synthase (EC 2.5.1.-), phytoene desaturase (EC 1.14.99.-), ξ-carotene desaturase (1.14.99.30), carotene isomerase, lycopene cyclase (EC 1.14.-.-), β-carotene hydroxylase (EC 1.14.13.-), zeaxanthin epoxidase (EC 1.14.-.-), neoxanthin synthase and (ii) “strigolactone biosynthetic enzymes” or “secondary carotenoid pathway enzymes” or “enzymes involved in carotenoid catabolism to strigolactone germination stimulants”, which are enzymes from and downstream of the branch point in the carotenoid pathway where germination stimulant formation branches away from the primary carotenoid/ABA biosynthetic pathway (see also FIG. 6), such as, but not limited to, carotenoid cleavage dioxygenases (CCD), 9-cis-epoxycarotenoid dioxygenase (NCED), cytochrome P450 hydroxylases, epoxidases, dehydrogenases, demethylase and the D-ring coupling enzyme. Similarly, the genes encoding these enzymes are referred to as “target genes”, “carotenoid pathway genes”, etc.

The terms “protein” or “polypeptide” are used interchangeably and refer to molecules consisting of a chain of amino acids, without reference to a specific mode of action, size, 3-dimensional structure or origin. A “fragment” or “portion” of a carotenoid cleavage dioxygenase (CCD) protein may thus still be referred to as a “protein”. An “isolated protein” is used to refer to a protein which is no longer in its natural environment, for example in vitro or in a recombinant bacterial or plant host cell. An enzyme is a protein comprising enzymatic activity.

The term “gene” means a DNA sequence comprising a region (transcribed region), which is transcribed into an RNA molecule (e.g. an mRNA) in a cell, operably linked to suitable regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′non-translated sequence comprising e.g. transcription termination sites.

A “chimeric gene” (or recombinant gene) refers to any gene, which is not normally found in nature in a species, in particular a gene in which one or more parts of the nucleic acid sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more coding sequences or to an antisense (reverse complement of the sense strand) or inverted repeat sequence (sense and antisense, whereby the RNA transcript forms double stranded RNA upon transcription).

“Expression of a gene” refers to the process wherein a DNA region, which is operably linked to appropriate regulatory regions, particularly a promoter, is transcribed into an RNA, which is biologically active, i.e. which is capable of being translated into a biologically active protein or peptide (or active peptide fragment) or which is active itself (e.g. in posttranscriptional gene silencing or RNAi). The coding sequence is preferably in sense-orientation and encodes a desired, biologically active protein or peptide, or an active peptide fragment. In gene silencing approaches, the DNA sequence is preferably present in the form of an antisense DNA or an inverted repeat DNA, comprising a short sequence of the target gene in antisense or in sense and antisense orientation. “Ectopic expression” refers to expression in a tissue in which the gene is normally not expressed. A “transcription regulatory sequence” is herein defined as a nucleic acid sequence that is capable of regulating the rate of transcription of a (coding) sequence operably linked to the transcription regulatory sequence. A transcription regulatory sequence as herein defined will thus comprise all of the sequence elements necessary for initiation of transcription (promoter elements), for maintaining and for regulating transcription, including e.g. attenuators or enhancers. Although mostly the upstream (5′) transcription regulatory sequences of a coding sequence are referred to, regulatory sequences found downstream (3′) of a coding sequence are also encompassed by this definition.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is preferentially (not necessarily exclusively) active in specific types of tissues or cells. For example, a root specific promoter is a promoter which is mainly (and preferably exclusively) active in the root tissue.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous and, where necessary to join two protein encoding regions, contiguous and in reading frame so as to produce a “chimeric protein”. A “chimeric protein” or “hybrid protein” is a protein composed of various protein “domains” (or motifs) which is not found as such in nature but which a joined to form a functional protein, which displays the functionality of the joined domains (for example DNA binding or repression leading to a dominant negative function). A chimeric protein may also be a fusion protein of two or more proteins occurring in nature. The term “domain” as used herein means any part(s) or domain(s) of the protein with a specific structure or function that can be transferred to another protein for providing a new hybrid protein with at least the functional characteristic of the domain.

The terms “target peptide” refers to amino acid sequences which target a protein to intracellular organelles such as vacuoles, plastids, preferably chloroplasts, mitochondria, leucoplasts or chromoplasts, the endoplasmic reticulum, or to the extracellular space (secretion signal peptide). A nucleic acid sequence encoding a target peptide may be fused (in frame) to the nucleic acid sequence encoding the amino terminal end (N-terminal end) of the protein or may replace part of the amino terminal end of the protein.

A “nucleic acid construct” or “vector” is herein understood to mean a man-made nucleic acid molecule resulting from the use of recombinant DNA technology and which is used to deliver exogenous DNA into a host cell. The vector backbone may for example be a binary or superbinary vector (see e.g. U.S. Pat. No. 5,591,616, US2002138879 and WO9506722), a co-integrate vector or a T-DNA vector, as known in the art and as described elsewhere herein, into which a chimeric gene is integrated or, if a suitable transcription regulatory sequence is already present, only a desired nucleic acid sequence (e.g. a coding sequence, an antisense or an inverted repeat sequence) is integrated downstream of the transcription regulatory sequence. Vectors usually comprise further genetic elements to facilitate their use in molecular cloning, such as e.g. selectable markers, multiple cloning sites and the like (see below).

A “host cell” or a “recombinant host cell” or “transformed cell” are terms referring to a new individual cell (or organism) arising as a result of at least one nucleic acid molecule, especially comprising a chimeric gene encoding a desired protein or a nucleic acid sequence which upon transcription yields an antisense RNA or an inverted repeat RNA (or hairpin RNA) for silencing of a target gene/gene family, having been introduced into said cell. The host cell may be any eukaryotic or prokaryotic cell e.g. a plant cell, microbial, insect or mammal (including human) cell. The host cell may contain the nucleic acid construct as an extra-chromosomally (episomal) replicating molecule, or more preferably, comprises the chimeric gene integrated in the nuclear or plastid genome of the host cell. Included are any derivatives of the host cell, such as tissues, whole organism, cell cultures, explants, protoplasts, further generations, etc. derived from the cell which retain the introduced gene.

In some embodiments the term “host” is used in the pathological sense and refers to the plant species which is infected by parasitic weed species, but this use of the term will be clear from the context.

The term “selectable marker” is a term familiar to one of ordinary skill in the art and is used herein to describe any genetic entity which, when expressed, can be used to select for a cell or cells containing the selectable marker. Selectable marker gene products confer for example antibiotic resistance, or herbicide resistance or another selectable trait such as a phenotypic trait (e.g. a change in pigmentation) or a nutritional requirement. The term “reporter” is mainly used to refer to visible markers, such as green fluorescent protein (GFP), eGFP, luciferase, GUS and the like. The term “ortholog” of a gene or protein refers herein to the homologous gene or protein found in another species, which has the same function as the gene or protein, but is (usually) diverged in sequence from the time point on when the species harbouring the genes diverged (i.e. the genes evolved from a common ancestor by speciation). Orthologs of the genes of interest may thus be identified in other plant, animal, bacterial or fungal species based on both sequence comparisons (e.g. based on percentages sequence identity over the entire sequence or over specific domains) and/or functional analysis.

The terms “homologous” and “heterologous” refer to the relationship between a nucleic acid or amino acid sequence and its host cell or host organism, especially in the context of transgenic cells/organisms. A homologous sequence is thus naturally found in the host species (e.g. a tomato plant transformed with a tomato gene), while a heterologous sequence is not naturally found in the host cell (e.g. a tomato plant transformed with a sequence from potato plants). Depending on the context, the term “homolog” or “homologous” may alternatively refer to sequences which are descendent from a common ancestral sequence (e.g. they may be orthologs).

“Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) Molecular Cloning: A Laboratory Manual, Second Edition, Cold Spring Harbor Laboratory Press, and Sambrook and Russell (2001) Molecular Cloning: A Laboratory Manual, Third Edition, Cold Spring Harbor Laboratory Press, NY.

“Sequence identity” and “sequence similarity” can be determined by alignment of two peptide or two nucleotide sequences using global or local alignment algorithms. Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length, maximizing the number of matches and minimises the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA or the open-source software Emboss for Windows (current version 2.7.1-07). Alternatively percent similarity or identity may be determined by searching against databases such as FASTA, BLAST, etc.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”, e.g. “a cell” refers also to several cells in the form of cell cultures, tissues, whole organism, etc. It is further understood that, when referring to “sequences” herein, generally the actual physical molecules with a certain sequence of subunits (e.g. amino acids) are referred to.

### DETAILED DESCRIPTION OF THE INVENTION

In a series of experiments with pathway inhibitors and maize mutants the present inventors surpisingly found that the germination stimulants of S. hermonthica present in the root exudates of maize, cowpea and sorghum are derived from the carotenoid biosynthetic pathway. This is unexpected, since the strigolactones have always been classified as sesquiterpenes, which is a principally different chemical class (see e.g. Akiyama et al., 2005, supra; Cook et al., 1972, supra; Butler, 1995, supra; Hauck et al., 1992, supra; Siame et al., 1993, supra; Muller et al., 1992, supra; Yokota et al., 1998, supra; Yoneyama et al., 2004, supra). Further, it was found that this also holds for the germination stimulant(s) of O. crenata in the root exudate of cowpea. For these three host species—maize, cowpea and sorghum—the germination stimulants have been isolated and identified to be strigolactones, viz. strigol, alectrol and sorgolactone (FIG. 1) (Hauck et al., 1992, supra; Muller et al., 1992, supra; Siame et al., 1993, supra). The present results with O. crenata suggest that this species also responds to a strigolactone germination stimulant. O. crenata is not a parasite of cowpea but parasitizes legumes in more-temperate climates such as North-Africa and Spain. The germination stimulant(s) of O. crenata has not been identified yet. Root exudates of cowpea readily induced germination of O. crenata and fluridone treatment decreased this germination by about the same percentage as for S. hermonthica. This makes it likely that the germination stimulants of Orobanche spp. that parasitise legumes in temperate regions of the world are also strigolactones and are hence also derived from the carotenoid pathway. This hypothesis is supported by the detection of orobanchol, alectrol and a third unidentified strigolactone in the legume red clover (Yokota et al., 1998, supra) and alectrol in cowpea (Muller et al., 1992, supra).

The fact that the present inventors demonstrate the carotenoid-origin of the germination stimulants for two parasitic plant species and three mono- and dicotyledonous hosts, and the (tentative) identification of strigolactones in the root exudates of other plant species such as red clover and tomato (Yokota et al., 1998, supra; Yoneyama et al., 2004, supra) suggest that carotenoid-derived germination stimulant formation occurs throughout the plant kingdom. This finding enables for the first time to effectively modify the levels of one or more germination stimulants produced by a plant cell, tissue, organ or whole plant, thereby modifying the resistance to one or more parasitic weed species (see further below).

The finding of the inventors that sorghum root exudate induced S. hermonthica germination is completely blocked by fluridone sheds an interesting light on the discussion about the “true” nature of the sorghum germination stimulant. Lynn and coworkers have claimed that sorgoleone is the natural sorghum germination stimulant but this was disputed by Zwanenburg and coworkers based a.o. on the low water solubility of dihydrosorgoleone (Butler, 1995, supra; Keyes et al., 2001, supra; Wigchert and Zwanenburg, 1999, supra). The finding herein makes it very likely that indeed the natural sorghum germination stimulant is a strigolactone.

The germination stimulants are produced in extremely low concentrations, which causes large difficulties for an analytical approach to discover the biosynthetic origin of the germination stimulants. The present inventors used a germination bioassay-guided approach herein to unravel the true biosynthetic pathway of germination stimulants. For these bioassays, the seeds of Orobanche and Striga spp. were preconditioned for a certain period of time at a suitable temperature such that the seeds become responsive to germination stimulants (Matusova et al., 2004, Seed Sci Res, 14, 335-344) (see Example 1). Seedlings of plant species to be investigated were grown in perlite, in sand or on filter paper, conditions under which the effect of inhibitors and transgenes can be investigated. After 1-2 weeks, seedlings were taken from the growing medium, carefully cleaned if necessary and then put into tap water in glass tubes for 6-24 hrs. After root exudate collection, root weight was determined and root exudates were diluted to equal root weight per volume concentrations. 50 μl of the test solutions were added to duplicate or triplicate disks containing preconditioned seeds and germination evaluated under a disecting microscope. The synthetic germination stimulant GR24 (0.01 and 0.1 mg.L⁻¹ GR24) as a positive and water as a negative control were included in each bioassay. Alternative bioassays used are the in situ assays described for rice (Example 3) and tomato (Example 7). In these bioassays, the germination of Striga and Orobanche seeds was evaluated by placing the seeds close to the roots of plants growing in sand (rice) or growing on filter paper (tomato).

In one embodiment of the invention the above bioassay, and its use for various purposes, such as testing and identifying or selecting plants (especially recombinant or mutant plants) for the production of germination stimulants, is provided. The assay may also be used to identify or select target genes and to verify the function of target genes, as described elsewhere herein or the use of mycorrhizae or chemicals to reduce germination stimulant production as described elsewhere herein. The bioassay preferably involves testing the germination of Orobanche and/or Striga seeds in the presence of roots, root exudates or root extracts and identifying a plant (or a transgene) which results in lower or higher germination compared to a control. Preferably, the roots, root exudates or root extracts are from a recombinant plant overexpressing a target gene or wherein a target gene is down-regulated.

For downregulated target genes (especially under control of a root-specific promoter), it is preferred that those plants are identified and/or selected of which the roots, root exudates or root extracts result in a significantly lower seed germination percentage of Orobanche and/or Striga seeds compared to a suitable control (e.g. a non-recombinant control or an empty-vectro control). A significantly lower germination is a reduced percentage of germination of Orobanche and/or Striga seeds by at least 20% compared to the control, preferably at least 30%, 40%, 50%, or more.

The carotenoid biosynthesis inhibitor fluridone effectively blocks the activity of phytoene desaturase which catalyses two desaturation steps converting phytoene to ξ-carotene and corresponds to the vp5 locus (Hable et al., 1998, Mol Gen Genet, 257, 167-176; Li et al., 1996, Plant Mol. Biol., 30, 269-279). The vp5 mutant of maize has a lesion in the phytoene desaturase gene (Hable et al., 1998, supra) and reduced Pds transcript in maize endosperm was detected in this mutant (Li et al., 1996, supra). The vp5 mutant results in lethality of the plant at the seedling stage. Surprisingly, both fluridone-treated maize and the vp5 mutant root exudates induced significantly lower germination of S. hermonthica (FIGS. 3, 5).

Both mevastatin and fosmidomycin also slightly, but not significantly, reduced root exudate induced germination (FIG. 3A). Both compounds have been demonstrated to be quite efficiently inhibiting the cytosolic or plastidic isoprenoid biosynthetic pathway, respectively (Laule et al., 2003, Proc Natl Acad Sci USA, 100, 6866-6871) and hence one might expect a clear effect of fosmidomycin, which in principle should also block carotenoid biosynthesis. However, fosmidomycin and mevastatin have also been used to prove exchange of isoprenoid intermediates between cytosol and cytoplasm, likely in the form of IPP (Botella-Pavia et al., 2004, Plant J, 40, 188-199; Hemmerlin et al., 2003, J Biol Chem, 278, 26666-26676). Recently, it was also shown that the exchange from cytosol to plastids occurs particularly in the dark (Botella-Pavia et al., 2004, supra). All this suggests that the inhibition by fosmidomycin of carotenoid formation was less effective than by fluridone, perhaps as a consequence of isoprenoid precursor supply from the cytosol. This assumption is supported by the phenotype of the treated seedlings. Fosmidomycin-treated seedlings did not exhibit the bleached phenotype that fluridone-treated seedlings showed. The modest inhibition of root exudate induced germination by mevastatin is less easy to explain. Possibly, inhibition of the cytosolic IPP formation also diminishes plastidic IPP concentrations either because there is no IPP export from the cytosol to the plastids anymore or because IPP is now exported more readily from the plastids into the cytosol. Alternatively, it could be that part of the germination stimulant biosynthesis occurs in the cytosol. In abscisic acid biosynthesis, xanthoxin is transported out of the plastids into the cytosol, where it is further processed to form ABA. Possibly, the strigolactone precursor is also exported to the cytosol after carotenoid cleavage. There, several additional enzymatic reactions would still be required (see below) and the D-ring would have to be coupled. The origin of the D-ring is as yet completely unknown, but it seems to be isoprenoid. It could arise from coupling of the tricyclic A-B-C skeleton to an isoprenoid unit such as dimethylallyl diphosphate or 4-hydroxydimethylallyl diphosphate by the action of a prenyl transferase. If this occurs in the cytosol then mevastatin treatment could also lead to a lower formation of germination stimulants, as the D-ring is required for biological activity (Wigchert and Zwanenburg, 1999, supra).

In addition to fluridone-treated maize seedlings, the root exudates of maize carotenoid mutants lw1, y10, al1y3, vp5 and y9 also induced lower germination of S. hermonthica seeds than their corresponding wild-type phenotype siblings. These results indicate that the biosynthetic pathway leading to the production of the strigolactone germination stimulant branches from the main carotenoid pathway below phytoene (vp5 mutant and fluridone) and ξ-carotene (y9 mutant). In maize, only four genes encoding early steps in carotenoid biosynthesis have been cloned and characterized so far. The maize y1 (yellow 1) gene encoding phytoene synthase, the enzyme that catalyse the first biosynthetic step specific to carotenoid production (Buckner et al., 1990, Plant Cell, 2, 867-876; Buckner et al., 1996, Genetics, 143, 479-488), the maize phytoene desaturase corresponding to the vp5 locus in maize (Hable et al., 1998, supra; Li et al., 1996, supra), the maize ξ-carotene desaturase corresponding to the vp9 (viviparous 9) locus (Luo and Wurtzel, 1999, Plant Physiol., 120, 1206; Matthews et al., 2003, J Exp Bot, 54, 2215-2230) and the Ps1 (Pink scutellum 1) gene corresponding to the vp7 locus encoding lycopene-β-cyclase (Singh et al., 2003, Plant Cell, 15, 874-884). The inventors' results in germination bioassays with root exudates of amitrole treated plants (FIGS. 2, 3C) confirmed that the branch point is below lycopene. Accumulation of lycopene in maize plants treated with amitrole and grown at 20° C. was observed (Dalla Vecchia et al., 2001, supra). In plants, the linear carotene lycopene is the precursor for cyclic carotenoids (reviewed by (Hirschberg, 2001, Curr Opin Plant Biol, 4, 210-218) at the branch point of two pathways. In one branch, the formation of a βionone ring at both ends of lycopene through the action of lycopene β-cyclase leads to the formation of β-carotene and its derivatives, including abscisic acid. In the other branch, the co-action of a lycopene ε-cyclase and lycopene β-cyclase leads to the formation of α-carotene and its derivatives, including lutein.

### Carotenoid Cleavage Enzymes

In a next step the present inventors tested the effect of naproxen, a putative inhibitor of epoxy-carotenoid cleavage (Lee and Milborrow, 1997, Aust J Plant Physiol, 24, 715-726) (FIG. 2). The formation of the germination stimulant of maize was indeed reduced by the action of naproxen (FIG. 3D). This suggests that carotenoid cleavage is involved in its biosynthesis, which is logical as the C40-carotenoids need to be cleaved in order to lead to the C14 (excluding the D-ring) strigolactones. Naproxen has been used to inhibit the 9-cis-epoxycarotenoid dioxygenase (NCED), a sub-category of the carotenoid cleavage dioxygenases (CCDs), that catalyses the oxidative cleavage of 9-cis-epoxy-carotenoids to apocarotenoids (C25) and xanthoxin (C15), the precursor of abscisic acid in higher plants (Schwartz, S. H. et al., 1997, Science, 276, 1872-1874). After cleavage, xanthoxin is exported to the cytosol where further conversion to abscisic acid takes place (FIG. 2) (Seo and Koshiba, 2002, Trends Plant Sci, 7, 41-48). The herein used bioassay with vp14, a mutant of NCED, confirmed the result obtained with naproxen. Also vp14 induced lower germination (FIG. 5). Both naproxen and vp14 were not fully effective in inhibiting root exudate induced germination but only gave about 44% inhibition compared with the corresponding control/wildtype seedlings (FIGS. 3D,5). This agrees quite well with results obtained by others. Lee and Milborrow reported that in a cell-free system of avocado, naproxen reduced radiolabel accumulation from [¹⁴C]-MVA in abscisic acid by 43% (Lee and Milborrow, 1997, supra). In the maize vp14 mutant, the mutation in NCED reduced abscisic acid accumulation in embryos by 40-60%, depending on embryo-developmental stage, and abscisic acid accumulation in water-stressed leaves by 45% (Tan et al., 1997, Proc Natl Acad Sci USA, 94, 12235-12240).

From the maize mutant vp14-2274, NCED was cloned and the substrate specificity characterized (Schwartz, S. H. et al., 2003, Biochim Biophys Acta-Gen Subjects, 1619, 9-14). The enzyme requires a 9-cis double bond adjacent to the site of cleavage (the ii-12 bond) and cleaves 9-cis-violaxanthin, 9-cis-neoxanthin and 9-cis-zeaxanthin (Schwartz et al., 2003, supra). However, the structural requirements in the carotenoid ring for cleavage have not been investigated and thus it is conceivable that NCED is non-specific and can cleave other 9-cis carotenoids such as 9-cis-β-carotene (FIG. 6). Other 9-cis-carotenoids, such as 9-cis-β-carotene and 9-cis-zeaxanthin have been reported to occur in a range of plant species (Ben-Amotz and Fishier, 1998, Food Chem, 62, 515-520). How these cis-isomers arise is unknown. However, recently it has been established that cis-isomers of early intermediates such as phytoene, phytofluene, ξ-carotene, neurosporene and lycopene are naturally occurring isomers in carotenoid biosynthesis (Isaacson et al., 2004, Plant Physiol., 136, 4246-4255; Park et al., 2002, Plant Cell, 14, 321-332). A carotene isomerase has been discovered that is responsible for the cis to trans isomerisation during the process of all-trans-lycopene formation. It is unclear whether cis-isomers of for example β-carotene arise from these early cis-intermediates or from trans to cis isomerisation in a later stage. Vp14 is expressed constitutively in maize embryos and roots and in Southern blots at least 9 bands were detected when hybridizing with vp14 cDNA (Tan et al., 1997, Proc Natl Acad Sci USA, 94, 12235-12240). This may also explain why the mutation in vp14 is not 100% effective in inhibiting abscisic acid (Tan et al., 1997, supra) nor germination stimulant formation (FIG. 5), as NCED family members may partly compensate for the NCED activity lost in vp14. NCED cDNAs have been cloned from several other plant species, such as Phaseolus vulgaris (Qin and Zeevaart, 1999, Proc Natl Acad Sci USA, 96, 15354-15361), cowpea (luchi et al., 2000, Plant Physiol., 123, 553-562), avocado (Chernys and Zeevaart, 2000, Plant Physiol., 124, 343-353) and Arabidopsis (Neill et al., 1998, J Exp Bot, 49, 1893-1894). Just like in maize, in all these species, NCEDs were found to belong to a gene family. Luchi et al. (Luchi et al., 2000, supra) characterized VuNCED1 in cowpea which was strongly induced by drought stress in leaves and stems but not in roots. Under stress conditions abscisic acid accumulated in a similar pattern, i.e. mainly in leaves and stems.

Several authors have also cloned CCDs from a range of plant species. For example, several CCDs were cloned from Arabidopsis (Schwartz, Steven H. et al., 2001, J Biol Chem, 276, 25208-25211; Schwartz, S. H. et al., 2004, J Biol Chem, 279, 46940-46945). AtCCD1 cleaves carotenoids at the 9-10 and 9′-10′ position. A homologue of AtCCD1 was postulated to catalyse the cleavage of a carotenoid precursor to a C14 dialdehyde which is probably the precursor of mycorradicin, the major component of yellow pigment observed in roots colonized by arbuscular mycorrhiza (Fester et al., 2002b, Planta, 216, 148-154; Schwartz et al., 2001, supra) that was first identified in maize roots (Klingner et al., 1995, Phytochemistry, 38, 53-55). The function of these apocarotenoids is unknown but they have been shown to be produced upon mycorrhizal colonization by a multitude of plant species such as maize and other cereals, Medicago truncatula, tomato and tobacco (Fester et al., 2002a, Plant Cell Physiol, 43, 256-265; Strack et al., 2003, J Chem Ecol, 29, 1955-1979; Walter et al., 2000, Plant J, 21, 571-578).

Biosynthetic Fate after Carotenoid Cleavage

The inhibitor of abscisic acid-aldehyde oxidation, sodium tungstate did not have an effect, but abscisic acid strongly reduced root exudate induced S. hermonthica germination (FIG. 3). This showed that the germination stimulants are not derived from intermediates below abscisic acid-aldehyde nor from abscisic acid itself. The reduction of root exudate induced germination by abscisic acid is most probably due to feed back inhibition by the exogenously applied abscisic acid on the carotenoid pathway. The levels of violaxanthin and β-carotene were indeed reduced by about 35 and 65%, respectively, in abscisic acid-treated roots and in the shoot a similar trend was observed (Table 1). Consequently the formation of germination stimulant branching from the carotenoid biosynthetic pathway is also decreased. Very little is known about the effects of exogenously applied abscisic acid on carotenoid and abscisic acid biosynthesis in roots and other non photosynthetic tissues. Feedback regulation of phytoene desaturase in green tissue by accumulation of abscisic acid, the end-product of the carotenoid pathway, was proposed (Corona et al., 1996, Plant J, 9, 505-512). In Arabidopsis seedlings, exogenously applied abscisic acid did not affect the transcript level of NCED (Xiong et al., 2001b, Plant Cell, 13, 2063-2083). In contrast, positive feedback regulation by abscisic acid at the transcriptional level was observed for AAO3 (abscisic acid aldehyde oxidase) (Seo et al., 2000, Proc Natl Acad Sci USA, 97, 12908-12913) and abscisic acid3 (a molybdenum cofactor sulfurase) (Xiong et al., 2001a, Dev Cell, 1, 771-781).

In rice, the inventors used in situ assays to assess the effect of carotenoid biosynthesis inhibitors and also the possibility to apply the inhibitors, in low, sublethal, concentrations, to the root or shoot and hence influence Striga germination. Irrigation with a low concentration of fluridone, significantly reduced the number of germinated/attached Striga seeds. Control experiments, where the synthetic germination stimulant GR24 was used to induce full germination, showed that fluridone was not affecting the attachment phase (but just germination) (data not shown). In a subsequent experiment fluridone was applied by spraying, and also here a strong, significant reduction in the number of germinated/attached seeds/tubercles of Striga was obtained even with very low concentrations. In both experiments, the concentrations of fluridone used were so low that bleaching of the leaves did not occur. Although the rice germination stimulant has sofar not been identified these results clearly demonstrate that they must also be strigolactones, and that hence rice—because the genome is sequenced, it can be easily transformed and because the inventors have herein developed a suitable bioassay to evaluate strigolactone formation on individual plants—is a suitable model to look for strigolactone biosynthetic pathway gene candidates. These experiments show, that herbicides which inhibit carotenoid biosynthesis can be used for significantly reducing the germination of parasitic seeds and that spraying or irrigating plants or soil with such herbicides at one or more time intervals is an effective way for reducing parasitic-weed induced yield losses of crop plants. Especially, the use of low amounts of inhibitors is preferred, so that no bleaching of the leaves occurs but still a significant reduction in the percentage of parasitic weed germination and/or attachment is seen compared to controls (see FIG. 7). Depending on the activity of the inhibitor, for example, amounts of less than 2 μM or less than 1 μM, such as equal to or less than 0.1 μM, 0.0 μM, 0.00 μM may be used.

The present results demonstrate that the strigolactone germination stimulants of S. hermonthica and O. crenata in the exudates of maize, sorghum and cowpea—strigol, sorgolactone and alectrol—and the unknown strigolactone germination stimulant of rice are derived from the carotenoid biosynthetic pathway. Considering the high structural similarity of orobanchol with these other strigolactones, also orobanchol must be derived from this pathway and this will also hold for the tentative strigol-analogues, recently reported for tomato (Yoneyama et al., 2004, supra). The results with the present bioassays with inhibitors and mutants suggest that the biosynthesis of germination stimulants branches off from the carotenoid pathway at an intermediate that is a product of NCED or CCD action. This may be xanthoxin but could more likely be an analogue derived from cleavage of other substrates such as 9-cis-β-carotene. A biogenetic scheme can be postulated starting from such a 9-cis-β-carotene cleavage products, but also from xanthoxin (FIG. 6). However, downstream derivatives of xanthoxin or alternative carotenoids could also serve as substrate in this biogenetic scheme.

Starting from 9-cis-β-carotene, C11-12 cleavage by a dioxygenase leads to a C15-aldehyde, which upon hydroxylation yields intermediate a (FIG. 6). Alternatively, xanthoxin, upon opening of the epoxide ring followed by protonation, elimination of water at C3 followed by hydrogenation, attack of water and loss of water could also lead to intermediate a. Oxidation followed by epoxidation and decarboxylation, protonation and elimination of water leads to intermediate b. This intermediate, upon attack of water at C7, cyclizes to intermediate c or its tautomeric aldehyde. After oxidation of the methyl at C9 to an acid function, a lactone ring will be formed with the C7 hydroxyl group to produce intermediate d. Alternatively, the methyl at C9 could already be oxidized to form a carboxyl group in compound a (not shown in FIG. 6). Instead of attack of water at C7, that carboxyl group could then attack the carbocation at C7 leading directly to intermediate d. As an alternative to lactone ring formation as described above, intermediate c could undergo keto-enol tautomerization, followed by oxidation to form a carboxyl group at C10, which can then form a lactone ring with the hydroxy at C7 (not shown in FIG. 6). Oxidation of the methyl at C9 to an aldehyde, would then also lead to intermediate d. From intermediate d, allylic hydroxylation in ring A or B or demethylation in ring A and coupling of the D-ring will lead to strigol, orobanchol and sorgolactone, respectively (FIG. 6). The structure of alectrol is still under debate. It is not unlikely that our biogenetic scheme may help to postulate a new structure for alectrol which should subsequently be proven using chemical synthesis. Of course the order of the reactions as proposed herein may in vivo be different. In addition, it is not unlikely that the D-ring is coupled to for example intermediate d before hydroxylation/demethylation to form the three different—and other—germination stimulants.

In conclusion, the present results with mutants and inhibitors, and the postulated biogenetic scheme lead to the conclusion that a carotenoid cleaving enzyme such as CCD or NCED, is involved in the biosynthesis of the strigolactone germination stimulants in hosts of Orobanche and Striga spp. After this cleavage step, a number of other enzymatic reactions, such as hydroxylation, epoxydation, oxidation, demethylation, D-ring coupling, etc are involved in the further modification of the primary apocarotenoid skeleton to the different strigolactones.

### Target Genes According to the Invention

In one embodiment of the invention a method for modulating (i.e. reducing or enhancing) the amount of germination stimulant produced by a plant cell, tissue or plant is provided (see further below). This method involves either expression vectors or gene silencing vectors, comprising a suitable promoter operably linked to one or more nucleic acid sequences, whereby the introduction into a host cell results in either overexpression of the recombinant gene or silencing of the endogenous target gene or gene family. In turn, the recombinant cell, tissue or plant produces either significantly enhanced amounts of one or more germination stimulants (overexpression) or significantly reduced amounts of one or more germination stimulants (downregulation), compared to the non-recombinant control (or control plants transformed with a control vector, e.g. an empty vector). Cell, tissues or plants with modified formation of germination stimulants can be easily selected using a germination bioassay, as described above and in the Examples.

As it was found that germination stimulants are synthesized via the carotenoid pathway, all nucleic acid sequences encoding carotenoid pathway enzymes (either primary carotenoid pathway enzymes or secondary carotenoid pathway enzymes, as defined) are target DNAs for genetic engineering, breeding or selection of plant cell, tissues or whole plants with reduced or increased germination stimulant formation. Moreover, it has been suggested that direction of radicle growth after germination of parasitic plant seeds is along the germination stimulant gradient (Dube and Olivier, 2001, supra). Therefore, in addition to a direct negative effect on germination, a reduced formation of germination stimulants may also contribute in reduced success of radicle growth in the right direction.

Suitable target enzymes of the primary carotenoid pathway are, but are not limited to, the enzymes indicated in FIG. 2, such as phytoene synthase, phytoene desaturase, ξ-carotene desaturase, carotene isomerase, lycopene cyclase, β-carotene hydroxylase, zeaxanthin epoxidase and neoxanthin synthase. The nucleic acid sequences may be derived from various sources, such as monocotyledonous or dicotyledonous plants, bacteria, fungi, etc. Nucleic acid sequences encoding such enzymes are widely available in the art and may be synthesized or may be easily cloned using existing DNA and protein information. For example, sequence information either from maize and other plant species is available in Genbank and other databases. Suitable examples are herein provided (nucleic acid sequence ID 1, 3, 5, 7, 9, 11, 13 and 15 and amino acid sequences 2, 4, 6, 8, 10, 12, 14 and 16; see sequence listing) and their use in the methods according to the invention is one embodiment. Obviously, sequences essentially similar to these (variants), such as for example orthologs from various species, and fragments of any of these sequences are also suitable.

A preferred embodiment is the use of these cDNAs or fragments thereof (e.g. sense and/or antisense fragments) in antisense or RNAi constructs (or vectors) to knock-down/downregulate the endogenous target gene(s) and thereby to reduce the formation of carotenoid intermediates supplying the substrate for germination stimulant formation. Preferably, these constructs are expressed in an organ-specific and/or development-specific way, such that the inhibition of carotenoid/ABA biosynthesis is restricted to time and place necessary to obtain resistance against parasitic plants and to avoid too much side-effects on mycorrhizal colonisation. Thus, in one embodiment an gene silencing vector is provided comprising a suitable promoter which is active in the host cell (preferably a tissue specific, e.g. root specific promoter) operably linked to a sense and/or antisense nucleic acid fragment of a carotenoid pathway gene. Hosts of parasitic plants (e.g. maize, tomato, sunflower, rice, etc) can be transformed with these constructs and primary, rooted, transformants easily assayed for their induction of germination of seeds of the corresponding parasitic plants using the bioassay described in this invention (Example 1). Successful inhibition of germination stimulant formation can thus be easily identified.

Also apocarotenoid pathway enzymes, i.e. the enzymes involved in cleavage and the postulated enzymes after cleavage, such as cytochrome P450 hydroxylases and epoxidases, dehydrogenaes, demethylase, a D-ring transferase etc are suitable targets for engineering, breeding or selection for reduced or increased germination stimulant formation. For genetic engineering a skilled person can clone the corresponding genes from parasitic plant hosts, such as maize, sunflower, tomato, tobacco, sorghum, millet, cowpea and rice (see below). Alternatively, the nucleic acid sequences provided herein e.g. the nucleic acid sequences encoding CCDs and NCEDs (sequence ID 17 and 19), and nucleic acid sequences encoding the proteins of sequence ID 18 and 20 from maize and the nucleic acid sequences encoding CCDs and NCEDs (sequence ID 21, 23, 25, 27, 29, 31, 33, 35, 37, 39, 41, 43, 45, 47 and 49) and nucleic acid sequences encoding the proteins of sequence ID 22, 24, 26, 28, 30, 32, 34, 36, 38, 40, 42, 44, 46, 48 and 50 from rice or sequences essentially similar thereto or encoding proteins with similar function or fragments thereof, may be used to make overexpression or gene silencing vectors. Several groups are investigating these enzymes usually in relation to the formation of flavour and fragrance compounds or pigments (Bouvier et al., 2003, Plant Cell, 15, 47-62) or in ABA biosynthesis (Schwartz et al., 2003, supra). Many CCDs and NCEDs have already been cloned or were identified from genome sequencing projects, eg on Arabidopsis and rice (Tan et al., 2003). In one embodiment of this invention a method for cloning of all rice CCDs and NCEDs is provided, which are used to knock-out their activity and to study the consequences of the knock-out for the induction of parasitic seed germination. In this way the rice gene that is involved in strigolactone formation is identified (see Example 7). Also, rice transposon and tDNA mutants in the CCDs and NCEDs can be used to this goal. From maize the present inventors have cloned two root-expressed target gene cDNAs (Sequence ID 17-20), as well as various CCD and NCED sequences from rice, which are one embodiment of the invention. A preferred embodiment is the use of these cDNAs in antisense or RNAi constructs to knock-down the cleavage of carotenoid intermediates supplying the apocarotenoid substrate for germination stimulant formation. Preferably, these constructs are expressed in an organ-specific and/or development-specific way, such that the inhibition of cleavage is restricted to time and place necessary to obtain resistance against parasitic plants.

In addition to the CCDs and NCEDs, there are several other enzyme classes that can be used as targets (FIG. 6). This concerns the genes encoding enzymes that are probably more specific only for germination stimulant formation such as the, but not limited to, as yet unidentified hydroxylases, epoxidases, of which some likely belong to the cytochrome P450s, dehydrogenases, demethylase and D-ring transferase. The genes encoding these enzymes can be readily cloned using molecular and biochemical methods known to persons skilled in the art and can then be used to modify germination stimulant formation as described herein. Strategies to clone these genes include subtractive techniques or transcriptomics approaches and thus identifying genes that are downregulated by mycorrhizal colonisation, as in the present invention we show that mycorrhizal colonisation reduces strigolactone formation (Example 3). Alternatively, the negative effect of phosphate on orobanchol production as reported by Yoneyama (personal communication) may be exploited in subtractive and/or transcriptomics approaches to pinpoint genes that are down-regulated by high phosphate and/or up-regulated by low phosphate and that fit into the herein presented biosynthetic scheme. The first cytochrome P450 encoding gene from this pathway may be cloned based on the homology expected to exist with MAX1, a cytochrome P450 from Arabidopsis and oxidising the carotenoid cleavage product of a CCD (CCD7/MAX3) (Booker et al., 2005, Developmental Cell 8, 443-449; Booker et al., 2004, Current Biology 14, 1232-1238). Cloning can also be achieved using enzyme purification and de novo sequencing, nucleic acid hybridization methods, degenerate primer PCR approaches or using genomics approaches such as cDNA micro-array analysis metabolomics and proteomics. Successful inhibition after transformation could be easily identified using the germination assays described in Examples 1, 3 and 7.

Thus, in one embodiment a method for identifying an enzyme involved in carotenoid catabolism to strigolactones (and cloning the gene encoding it and verifying its function) is provided, comprising the steps of
a) identifying a gene which is downregulated by mycorrhizal colonization and/or by high phosphate and/or which is up-regulated by low phosphate (phosphate limitation); and
b) transforming a plant, plant cell or plant tissue with the nucleotide sequence operably linked to a promoter active in plants (i.e. with an expression or silencing construct) and obtaining a transformed plant thereform; and optionally
c) testing the germination of Orobanche and/or Striga seeds in the presence of roots, root exudates or root extracts of the recombinant plant (e.g. using the bioassay described); and optionally
d) selecting a plant which results in lower or higher seed germination compared to a control plant or selecting the gene whose downregulation/silencing in the recombinant plant results in significantly lower or whose overexpression results in significantly higher seed germination compared to the control plant for further use, e.g. for generating a transgenic plant or for identifying herbicides or compounds which specifically inhibit the activity of the enzyme encoded by the gene.

In another preferred embodiment, the above mentioned known or still to be obtained target gene sequences can be used in TILLING approaches (Targeting Induced Local Lesions IN Genomics; McCallum et al., 2000, Nat Biotech 18:455, and McCallum et al. 2000, Plant Physiol. 123, 439-442, both incorporated herein by reference), where in induced-mutant collections of plants or plant tissues (e.g. seeds), are screened for mutations in target genes using PCR-based methods. Such an induced-mutant collection can also be used to screen more untargeted for mutants affected in any of the genes described above, and the genes can then be cloned. Also transposon-knock out mutant collections provide a suitable source to look for mutants affected in any of the target genes. Because of the transposon, the affected genes can easily be cloned. Plants can be grown in a high-throughput manner, and root exudates collected after 10 days and tested for induction of germination (see Example 1) or under greenhouse or field conditions on soil infested with parasitic plant seeds. These mutants can then be characterised and the mutated genes cloned and used to knock out germination stimulant formation in crop species. Another preferred embodiment is the selection of natural (spontaneous) mutants in crop species affected in carotenoid formation or cleavage in their roots, whereby the plant roots produce either enhanced or reduced amounts of one or more germination stimulants. Such plants can be obtained by screening a plurality of plants, e.g. germbank collection, for modified expression of target genes and/or modified germination stimulant levels using e.g. the germination bioassays described in the present invention (Examples 1, 3 and 7).

As an alternative to knocking out enzymes from the germination stimulant pathway, overexpression of key-enzymes of competing pathways to channel away substrate can be considered as a strategy to reduce germination stimulant formation. Also, the strong inhibiting effect of externally applied ABA can be used as a control strategy. Alternative production pathways for ABA exist in micro-organisms (Siewers et al., 2004, Appl. Environ. Microbiol, 70, 3868-3876). There, ABA biosynthesis proceeds from farnesyl diphosphate and not geranylgeranyl diphosphate. In a preferred embodiment such microbial ABA biosynthesis is introduced into parasitic plant hosts, preferably with root-specific expression and preferably under the control of gene promoters involved in normal ABA-biosynthesis. This will lead to the formation of ABA and subsequent feedback inhibition of the endogenous carotenoid/ABA biosynthetic pathway as demonstrated in FIG. 3 and Table 1, and hence to a reduction in germination stimulant formation. More preferred, this could be combined with knocking out carotenoid/ABA biosynthesis in roots, and hence to an even more efficient reduction in germination stimulant formation.

Alternatively, ABA catabolism in roots could be blocked, also leading to feed-back inhibition of the carotenoid/ABA pathway. Enzymes responsible for catabolism of ABA are known, for example the cytochrome P450 enzyme ABA 8′-hydroxylase, but also other catabolic pathways exist, such as 4′-reduction, 7′-hydroxylation and conjugation (Cutler and Krochko, 1999). In a preferred embodiment these catabolic enzymes are knocked-out preferably with root-specific expression. This will inhibit the catabolism of ABA and hence will lead to feedback inhibition of the endogenous carotenoid/ABA biosynthetic pathway as demonstrated in FIG. 3 and Table 1, and hence to a reduction in germination stimulant formation.

In another preferred embodiment chemicals that inhibit the activity of any of the target enzymes are used to block germination stimulant formation. Examples of this are—but are not limited to—fluridone, norflurazone, isoxaflutole, flurtamone, clomazone, fluorochloridone, pyridazinone, nicotinanilide, amitrole, naproxen or abamine, or cheap analogs of this, inhibitors of P450s or cheap analogs of ABA that very effectively blocks germination stimulant formation (FIG. 3). In the present invention we show that sub-lethal concentrations of fluridone—applied to the soil or sprayed on the leafs of the host plant, can be used to reduce germination stimulant formation and hence infection with parasitic weeds (Example 3).

In another preferred embodiment, to make the resistance mechanism based on reduced germination stimulant formation durable it is combined with other resistance mechanisms. For example, a low production of germination stimulants is combined with a parasite-inducible toxin such as the parasite-inducible promoter-sarcotoxin IA (WO 02/094008). The transgenic tobacco plants described in that invention are protected against Orobanche infection but only to a limited level. In combination with the present invention a more solid and durable resistance may be obtained, not only in tobacco but in all plant species that are parasitised by Orobanche and Striga spp.

Resistance levels of recombinant plants compared to controls can be tested in in vitro assays, green-house bioassays or preferably in field trials with a high parasitic plant pressure. “Resistance” refers herein to both a significant reduction in damage caused when exposed to the parasitic plant, as well as a complete absence of damage. Resistant plants have the advantage that less or no chemicals need to be used to protect the crop plants. Recombinant or mutant plants according to the invention produce at least less germination stimulants and have enhanced resistance to one or more parasite species compared to control plants. Especially, enhanced resistance against S. hermonthica, S. gesneroides, S. asiatica, S. aspera, S. forbesii, O. ramosa, O. crenata, O. cernua, O. cumana and O. aegyptiaca are provided.

In another embodiment, the fact that plants respond upon mycorrhization with reduced germination stimulant formation is used to control Orobanche and Striga spp. Most agricultural plants form arbuscular mycorrhizas, a beneficial relationship between plant roots and certain root-inhabiting fungi. Interestingly, two groups have reported that mycorrhiza can reduce Striga infection of sorghum and maize (Gworgwor and Weber, 2003, supra; Lendzemo, 2004, PhD thesis, supra; Lendzemo and Kuyper, 2001, supra). However, it was not known that mycorrhiza reduce the formation of germination stimulants. Also, it has been shown that micro-organisms may produce ABA and this has also been hypothesised to be true for mycorrhiza (Cutler and Krochko, 1999, Trends Plant Sci, 4, 472-478; Hartung and Gimmler, 1994, Prog Bot, 55, 157-173). In the present invention it was found that mycorrhizal colonisation reduces the production of germination stimulants (Example 4). This relationship now enables the inventors to explain and therefore also exploit the positive effect of mycorrhiza on parasitic plant infection. For example, much simpler and more efficient bioassays are provided herein, based on the finding that mycorrhiza reduce the formation of germination stimulants in the roots. Such bioassays were not possible before.

Without limiting the scope of the invention, a possible explanation is that due to the formation of mycorrhiza-specific apocarotenoids or ABA the formation of the Striga germination stimulant is reduced. Apocarotenoid formation (probably from β-carotene) may be competing for substrate with germination stimulant formation. Alternatively, mycorrhizal colonisation may be down-regulating the strigolactone production pathway. Interestingly, the apocarotenoids have been shown to be produced upon mycorrhization in a number of Orobanche hosts as well, such as Medicago truncatula, tomato and tobacco (Fester et al., 2002a, supra; Fester et al., 2002b, supra; Strack et al., 2003, supra; Walter et al., 2000, supra). In the present invention evidence is provided that also the germination stimulants for Orobanche spp. are derived from the carotenoid/ABA pathway. This is for example clear from the identification of strigol-analogs in tomato (Yoneyama et al., 2004, supra) and the present results with O. crenata (see above and FIG. 3). In one embodiment of the invention this knowledge is used to optimise this positive interaction between mycorrhizae and parasitic weed hosts by screening for successful host-mycorrhizal species combinations in which maximal reduction in germination stimulant formation is obtained using the bioassays described in the present invention (e.g. Example 4).

The fact that in for example tomato the germination stimulants are carotenoid-derived and that tomato is a host for mycorrhizae can now be used to investigate whether selective mycorrhization of tomato could be used as a control measure and to select suitable mycorrhizae-host combinations. This will also hold for many other crop species that can be colonised by mycorrhizae and are hosts of Orobanche spp., such as sunflower, and crop species from the Fabaceae and Solanaceae (such as tobacco and potato) (Fester et al., 2002b, supra).

Thus, in one embodiment a method for identifying and selecting a mycorrhiza -parasitic weed host plant combination, which results in a significant reduction of strigolactone germination stimulants in the mycorrhiza colonized plant is provided, comprising the steps of a) inoculating a plurality of parasitic weed host species and/or varieties with one or more mycorrhizal species and b) testing the germination of Orobanche and/or Striga seeds in the presence of roots, root exudates, or root extracts from the mycorrhiza-colonized plants (i.e. using a germination bioassay as described elsewhere herein) and c) identifying and optionally selecting the mycorrhiza-parasitic weed host plant combination which results in significantly lower seed germination compared to a suitable control, such as the plant lacking mycorrhiza or a plant-mycorrhiza combination inducing a specific percentage of parasitic seed germination. The method above is, thus, also a method for selecting plant host-mycorrhizae species combinations having enhanced resistance to one or more Orobanche and/or Striga species. Also provided is the use of mycorrhizae species for reducing germination stimulant production of an Orobanche and/or Striga host species and, thus, for enhancing resistance of the host plant to one or more Orobanche and/or Striga species. The host species or varieties may be any species which is a host of one or more parasitic weed species. In the bioassay, a reduction in the percentage of seed germination of at least 5%, 10%, 20%, 30%, 40%, 50%, or more compared to the control is a significant reduction. See FIG. 8 and Example 4.

In another embodiment, recombinant plant cells, tissues and organs overexpression one or more target genes are provided (see further below). This can be done by transforming so-called catch and trap crop species, i.e. crops that are removed after they have induced a lot of parasitic plant germination (catch crops) or crops that do induce germination but are no host to the parasite that is present. However, also in host crops, a very high germination stimulant production may be beneficial, if this induces germination (only) at sufficient distance from the root, so that the germinating radicle can not reach the host root. In addition, overexpression of strigolactone formation may improve mycorrhizal colonisation efficiency.

Also provided are nucleic acid sequences (genomic DNA, cDNA, RNA) encoding target genes (including any chimeric or hybrid genes). Due to the degeneracy of the genetic code various nucleic acid sequences may encode the same amino acid sequence, e.g. various nucleic acid sequences encode the proteins depicted in Seq Id No's 2, 4, 6, 8, 10, 12, 14, 16, 18, 20, 22, 24, 26, 28, 30, 32, 34, 36, 38, 40, 42, 44, 46, 48 and 50. SEQ ID NO 1 depicts the Zea mays phytoene synthase 1 cDNA. SEQ ID NO 2 depicts the Zea mays phytoene synthase 1 protein. SEQ ID NO 3 depicts the Zea mays phytoene desaturase cDNA. SEQ ID NO 4 depicts the Zea mays phytoene desaturase protein. SEQ ID NO 5 depicts the Zea mays ξ-carotene desaturase cDNA. SEQ ID NO 6 depicts the Zea mays ξ-carotene desaturase protein. SEQ ID NO 7 depicts the Lycopersicon esculentum carotene isomerase cDNA. SEQ ID NO 8 depicts the Lycopersicon esculentum carotene isomerase protein. SEQ ID NO 9 depicts the Zea mays lycopene-β-cyclase cDNA. SEQ ID NO 10 depicts the Zea mays lycopene-β-cyclase protein. SEQ ID NO 11 depicts the Oryza sativa β-carotene hydroxylase cDNA. SEQ ID NO 12 depicts the Oryza sativa β-carotene hydroxylase protein. SEQ ID NO 13 depicts the Oryza sativa zeaxanthin epoxidase cDNA. SEQ ID NO 14 depicts the Oryza sativa zeaxanthin epoxidase protein. SEQ ID NO 15 depicts the Solanum tuberosum neoxanthin synthase cDNA. SEQ ID NO 16 depicts the Solanum tuberosum neoxanthin synthase protein. SEQ ID NO 17 depicts the Zea mays vp14=9-cis-epoxycarotenoid dioxygenase (NCED) cDNA. SEQ ID NO 18 depicts the Zea mays vp14=9-cis-epoxycarotenoid dioxygenase (NCED) protein. SEQ ID NO 19 depicts the Zea mays carotenoid cleavage dioxygenase 1 cDNA (CCD1). SEQ ID NO 20 depicts the Zea mays carotenoid cleavage dioxygenase 1 protein (CCD1). SEQ ID NO 21: Oryza sativa 9-cis-epoxycarotenoid dioxygenase (NCED-3) cDNA. SEQ ID NO 22 depicts the Oryza sativa 9-cis-epoxycarotenoid dioxygenase (NCED-3) protein. SEQ ID NO 23 depicts the Oryza sativa 9-cis-epoxycarotenoid dioxygenase (NCED-5) cDNA. SEQ ID NO 24 depicts the Oryza sativa 9-cis-epoxycarotenoid dioxygenase (NCED-5) protein. SEQ ID NO 25 depicts the Oryza sativa 9-cis-epoxycarotenoid dioxygenase (NCED-4) cDNA. SEQ ID NO 26 depicts the Oryza sativa 9-cis-epoxycarotenoid dioxygenase (NCED-4) protein. SEQ ID NO 27 depicts the Oryza sativa 9-cis-epoxycarotenoid dioxygenase (NCED-1) cDNA. SEQ ID NO 28 depicts the Oryza sativa 9-cis-epoxycarotenoid dioxygenase (NCED-1) protein. SEQ ID NO 29 depicts the Oryza sativa 9-cis-epoxycarotenoid dioxygenase (NCED-2) cDNA. SEQ ID NO 30 depicts the Oryza sativa 9-cis-epoxycarotenoid dioxygenase (NCED-2) protein. SEQ ID NO 31 depicts the Oryza sativa 9-cis-epoxycarotenoid dioxygenase (NCED-like) cDNA. SEQ ID NO 32 depicts the Oryza sativa 9-cis-epoxycarotenoid dioxygenase (NCED-like) protein. SEQ ID NO 33 depicts the Oryza sativa lignostilbene 1 cDNA. SEQ ID NO 34 depicts the Oryza sativa lignostilbene 1 protein. SEQ ID NO 35 depicts the Oryza sativa carotenoid cleavage dioxygenase 8 (CCD8) cDNA. SEQ ID NO 36 depicts the Oryza sativa carotenoid cleavage dioxygenase 8 (CCD8) protein. SEQ ID NO 37 depicts the Oryza sativa neoxanthin 1 cleavage enzyme cDNA. SEQ ID NO 38 depicts the Oryza sativa neoxanthin 1 cleavage enzyme protein. SEQ ID NO 39 depicts the Oryza sativa neoxanthin 2 cleavage enzyme cDNA. SEQ ID NO 40 depicts the Oryza sativa neoxanthin 2 cleavage enzyme protein. SEQ ID NO 41 depicts the Oryza sativa lignostilbene 2 cDNA. SEQ ID NO 42 depicts the Oryza sativa lignostilbene 2 protein. SEQ ID NO 43 depicts the Oryza sativa crocetin cDNA. SEQ ID NO 44 depicts the Oryza sativa crocetin protein. SEQ ID NO 45 depicts the Oryza sativa dioxygenase cDNA. SEQ ID NO 46 depicts the Oryza sativa dioxygenase protein. SEQ ID NO 47 depicts the Oryza sativa carotenoid cleavage dioxygenase 7 (CCD7) cDNA. SEQ ID NO 48 depicts the Oryza sativa carotenoid cleavage dioxygenase 7 (CCD7) protein. SEQ ID NO 49 depicts the Oryza sativa carotenoid cleavage dioxygenase 1 (CCD1) cDNA. SEQ ID NO 50 depicts the Oryza sativa carotenoid cleavage dioxygenase 1 (CCD1) protein.

The nucleic acid sequences provided herein include naturally occurring, artificial or synthetic nucleic acid sequences. Included are also sequences generated from the provided sequences by e.g. gene shuffling methods as described in U.S. Pat. No. 5,811,238, WO97/20078, U.S. Pat. No. 6,180,406 and U.S. Pat. No. 6,117,679, which encode target proteins comprising higher or modified catalytic activity and methods of using the nucleic acid sequences of the invention for generating such “evolved” sequences. It is understood that when sequences are depicted as DNA sequences while RNA is referred to, the actual base sequence of the RNA molecule is identical with the difference that thymine (T) is replace by uracil (U).

Also included are variants and fragments of any of the target gene nucleic acid sequences, such as nucleic acid sequences hybridizing to target gene nucleic acid sequences under stringent hybridization conditions as defined, and fragments of these. Variants of target gene nucleic acid sequences also include essentially similar nucleic acid sequences, which have a nucleotide sequence identity (see definitions) to any target nucleic acid sequence, especially to any one of SEQ ID NO: 1, 3, 5, 7, 9, 11, 13, 15, 17, 19, 21, 23, 25, 27, 29, 31, 33, 35, 37, 39, 41, 43, 45, 47 and 49, of at least 30%, 40%, 50%, 60%, 70%, 80%, 85%, 90%, 95%, 98%, 99% or more. Further, variants of target nucleic acid sequences also include nucleic acid sequence encoding any target protein according to the invention, especially any one of the proteins of SEQ ID NO: 2, 4, 6, 8, 10, 12, 14, 16, 18, 20, 22, 24, 26, 28, 30, 32, 34, 36, 38, 40, 42, 44, 46, 48 and 50, and variants of any of these proteins. Variants of target proteins (i.e. proteins essentially similar to any of the target proteins) are proteins having an amino acid sequence identity (see definitions) of at least 30%, 40%, 50%, 60%, 70%, 80%, 85%, 90%, 95%, 98%, 99% or more to the target protein, especially to any one of the proteins of SEQ ID NO: 2, 4, 6, 8, 10, 12, 14, 16, 18, 20, 22, 24, 26, 28, 30, 32, 34, 36, 38, 40, 42, 44, 46, 48 and 50.

It is clear that many methods can be used to identify, synthesise or isolate variants or fragments of target gene nucleic acid sequences, such as nucleic acid hybridization, PCR technology, in silico analysis and nucleic acid synthesis, and the like.

The codon usage may be adapted to plant genes native to the plant genus or species of interest (Bennetzen & Hall, 1982, J. Biol. Chem. 257, 3026-3031; Itakura et al., 1977 Science 198, 1056-1063) using available codon usage tables (e.g. more adapted towards expression in cotton, soybean corn or rice). Codon usage tables for various plant species are published for example by Ikemura (1993, In “Plant Molecular Biology Labfax”, Croy, ed., Bios Scientific Publishers Ltd.) and Nakamura et al. (2000, Nucl. Acids Res. 28, 292.) and for various organisms in the major DNA sequence databases (e.g. EMBL at Heidelberg, Germany). Accordingly, synthetic DNA sequences can be constructed so that the same or substantially the same proteins are produced. Several techniques for modifying the codon usage to that preferred by the host cells can be found in patent and scientific literature. The exact method of codon usage modification is not critical for this invention. Likewise, codon usage of a monocot derived target gene may be (partially) adapted to a dicot preferred codon usage for expression in dicots, and vice versa (see Batard et al. 2000, Arch Biochem Biophys 379, 161-169).

Small modifications to a DNA sequence such as described above can be routinely made, i.e., by PCR-mediated mutagenesis (Ho et al., 1989, Gene 77, 51-59., White et al., 1989, Trends in Genet. 5, 185-189). More profound modifications to a DNA sequence can be routinely done by de novo DNA synthesis of a desired coding region using available techniques.

Also, the target gene nucleic acid sequences can be modified so that the N-terminus of the proteins has an optimum translation initiation context, by adding or deleting one or more amino acids at the N-terminal end of the protein. Often it is preferred that the proteins of the invention to be expressed in plants cells start with a Met-Asp or Met-Ala dipeptide for optimal translation initiation. An Asp or Ala codon may thus be inserted following the existing Met, or the second codon can be replaced by a codon for Asp (GAT or GAC) or Ala (GCT, GCC, GCA or GCG). The DNA sequences may also be modified to remove illegitimate splice sites.

### Target Gene Silencing Methods and Recombinant Plants

It is an object of the invention to provide a method for making a recombinant plant having one or more tissues (especially roots) which produce reduced amounts of one or more strigolactone germination stimulants compared to controls. This method comprises the steps of:
(a) generating a gene silencing vector comprising a promoter sequence operably linked to a sense and/or antisense nucleic acid sequence of a carotenoid pathway gene (target gene),
(b) transforming a plant or plant cell with the vector of step a), and
(c) regenerating a plant comprising at least one tissue which produces (significantly) reduced amounts of strigolactone germination stimulants compared to control plants.

Preferably, the root tissue or part of the root tissue produces (significantly) reduced amounts of the target enzyme (or family) and thus also of one or more strigolactone germination stimulants.

Also provided is a recombinant plant, plant cell or tissue comprising, integrated in its genome, a sense and/or antisense fragment of a nucleic acid sequence encoding a a carotenoid pathway enzyme operably linked to a promoter and optionally a 3′ non-translated nucleic acid sequence comprising a polyadenylation signal, whereby the recombinant plant, plant cell or tissue produces (significantly) reduced amounts of one or more strigolactone germination stimulants compared to a control plant, plant cell or tissue.

Preferably, if the target gene is a gene family, the whole gene family is silenced. Effective silencing can be analysed by for example determining the mRNA and/or protein levels of the target gene being produced. Silencing results in plants and/or tissues which produce significantly reduced amounts of the target-gene-derived product, the strigolactones. Any plant that is parasitised by Orobanche or Striga spp. may be a suitable host for transformation in order to silence germination stimulant formation. Suitable host plants are for example monocotyledonous plants or dicotyledonous plants, for example maize/corn (Zea species), pearl millet (Pennisetum spp. e.g. P. glaucum), rice (Oryza species, e.g. O. sativa indica cultivar-group or japonica cultivar-group), sorghum (Sorghum bicolor), soybean (Glycine spp, e.g. G. max), sunflower (Helianthus annuus), Brassica spp. (e.g. B. napus, B. juncea, B. oleracea, B. rapa, etc), tobacco (Nicotiana species), alfalfa (Medicago sativa), forage grasses, vegetable species, such as tomato (Lycopersicon ssp e.g. Lycopersicon esculentum), potato (Solanum tuberosum, other Solanum species), eggplant (Solanum melongena), peppers (Capsicum annuum, Capsicum frutescens), beans (e.g. Phaseolus species), zucchini, cucumber, melon, squash, artichoke, asparagus, broccoli, garlic, leek, lettuce, onion, radish, turnip, Brussels sprouts, carrot, cauliflower, chicory, celery, spinach, endive, fennel, beet, parsnip, herbs (mint, parsley, basil, thyme, etc.), other legumes such as pea, vetch, broadbean, faba bean, lentil, chickpea, cowpea, groundnut, bambara groundnut, fibre species e.g. flax (Linum usitatissimum) and hemp (Cannabis sativa).

Plant species transformed with a gene silencing construct under control of a suitable promoter, such as constitutive or a root specific promoter induce significantly lower germination of parasitic weed seeds due to a significant reduction (or complete absence) of strigolactones. A “significant reduction” (or “significantly reduced”) refers herein to a reduction of at least 5%, 10%, 20%, 30%, 40%, 50%, 70%, 80%, 90%, 95% or 100% less strigolactone(s) being produced, than produced by the control plant/cell/tissue. It is understood that endogenous expression of other enzymes involved in strigolactone biosynthesis or catabolism may additionally be silenced using an appropriate gene silencing construct, for example endogenous P450s may be silenced. The reduction in strigolactone production is easily assayed by, for example, using a germination bioassay, as described in the Examples. This assay is an indirect assay which however, due to its sensitivity, provides a good indication of the amounts of germination inhibitors being produced. Thus, the percentage of germination of the parasitic seeds is preferably at least 5%, 10%, 20%, 30%, 40%, 50%, 70%, 80%, 90%, 95% or 100% less in recombinant cells, tissues or plants, than in control plant/cell/tissue. Alternatively, biochemical or molecular assays may be used.

“Gene silencing” refers to the down-regulation or complete inhibition of gene expression of one or more target genes. The use of inhibitory RNA to reduce or abolish gene expression is well established in the art and is the subject of several reviews (e.g Baulcombe, 1996, Plant Cell 8: 1833-1844, Stam et al., 1997, Ann. Botan. 79:3-12, Depicker and Van Montagu, 1997, Curr. Opin. Cell. Biol. 9: 373-382). There are a number of technologies available to achieve gene silencing in plants, such as chimeric genes which produce antisense RNA of all or part of the target gene (see e.g. EP 0140308 B1, EP 0240208 B1 and EP 0223399 B1), or which produce sense RNA (also referred to as co-suppression), see EP 0465572 B1.

The most successful approach so far has however been the production of both sense and antisense RNA of the target gene (“inverted repeats”), which forms double stranded RNA (dsRNA) in the cell and silences the target gene. Methods and vectors for dsRNA production and gene silencing have been described in EP 1068311, EP 983370 A1, EP 1042462 A1, EP 1071762 A1 and EP 1080208 A1. A vector according to the invention may therefore comprise a transcription regulatory region which is active in plant cells operably linked to a sense and/or antisense DNA fragment of a target gene according to the invention. Generally short (sense and antisense) stretches of the target gene sequence, such as 17, 18, 19, 20, 21, 22 or 23 nucleotides of coding or non-coding sequence are sufficient. Longer sequences can also be used, such as about 100, 200, 250, 300, 500 nucleotides or more. Thus, sense and/or antisense fragments of these lengths of any of the target nucleic acids are provided. Preferably, the short sense and antisense fragments are separated by a spacer sequence, such as an intron, which forms a loop (or hairpin) upon dsRNA formation. Any short stretch of a target gene, especially of SEQ ID NO: 1, 3, 5, 7, 9, 11, 13, 15, 17, 19, 21, 23, 25, 27, 29, 31, 33, 35, 37, 39, 41, 43, 45, 47 and 49 or of any nucleic acid sequence encoding SEQ ID NO: 2, 4, 6, 8, 10, 12, 14, 16, 18, 20, 22, 24, 26, 28, 30, 32, 34, 36, 38, 40, 42, 44, 46, 48 and 50 (or any sequence essentially similar to these sequences ) may be used to make a target gene silencing vector and a transgenic plant in which one or more target genes are silenced in all or some tissues or organs. A convenient way of generating hairpin constructs is to use generic vectors such as pHANNIBAL and pHELLSGATE, vectors based on the Gateway® technology (see Wesley et al. 2004, Methods Mol. Biol. 265:117-30; Wesley et al. 2003, Methods Mol. Biol. 236:273-86 and Helliwell & Waterhouse 2003, Methods 30(4):289-95.), all incorporated herein by reference.

By choosing conserved nucleic acid parts of the target genes, target gene family members in a host plant can be silenced. Encompassed herein are also transgenic plants comprising a transcription regulatory element operably linked to a sense and/or antisense DNA fragment of a target gene and exhibiting a target gene silencing phenotype (i.e. reduced germination stimulant(s) being produced). Gene silencing constructs may also be used in reverse genetic approaches, to elucidate or confirm the function of a target gene or gene family in a host species.

The construction of chimeric genes and vectors for, preferably stable, introduction of the nucleic acid sequences into the genome of host cells is generally known in the art. To generate a chimeric gene the sense and/or antisense nucleic acid sequence (or, in overexpression approaches, the nucleic acid sequence encoding a target protein) according to the invention is operably linked to a promoter sequence, suitable for expression in the host cells, using standard molecular biology techniques. The promoter sequence may already be present in a vector so that the target gene nucleic acid sequence is simply inserted into the vector downstream of the promoter sequence. The vector is then used to transform the host cells and the chimeric gene is inserted in the nuclear genome or into the plastid, mitochondrial or chloroplast genome and expressed there using a suitable promoter (e.g., Mc Bride et al., 1995 Bio/Technology 13, 362; U.S. Pat. No. 5,693,507). In one embodiment a chimeric gene comprises a suitable promoter for expression in plant cells, operably linked thereto a nucleic acid sequence suitable for silencing the target gene(s) or encoding a functional target protein according to the invention (overexpression, see below), optionally followed by a 3′nontranslated nucleic acid sequence.

The target gene nucleic acid sequence, preferably the target chimeric gene, comprising the sense and/or antisense nucleic acid sequence (or encoding a functional target protein in overexpression approaches as described below), can be stably inserted in a conventional manner into the nuclear genome of a single plant cell, and the so-transformed plant cell can be used in a conventional manner to produce a transformed plant that has an altered phenotype due to the presence of the gene silencing nucleic acid sequence (or target protein) in certain cells at a certain time. In this regard, a T-DNA vector, comprising a nucleic acid sequence encoding a target protein, in Agrobacterium tumefaciens can be used to transform the plant cell, and thereafter, a transformed plant can be regenerated from the transformed plant cell using the procedures described, for example, in EP 0 116 718, EP 0 270 822, PCT publication WO84/02913 and published European Patent application EP 0 242 246 and in Gould et al. (1991, Plant Physiol. 95, 426-434). The construction of a T-DNA vector for Agrobacterium mediated plant transformation is well known in the art. The T-DNA vector may be either a binary vector as described in EP 0 120 561 and EP 0 120 515 or a co-integrate vector which can integrate into the Agrobacterium Ti-plasmid by homologous recombination, as described in EP 0116 718.

Preferred T-DNA vectors each contain a promoter operably linked to the target gene (sense and/or antisense) nucleic acid sequence between T-DNA border sequences, or at least located to the left of the right border sequence. Border sequences are described in Gielen et al. (1984, EMBO J. 3, 835-845). Of course, other types of vectors can be used to transform the plant cell, using procedures such as direct gene transfer (as described, for example in EP 0 223 247), pollen mediated transformation (as described, for example in EP 0 270 356 and WO85/01856), protoplast transformation as, for example, described in U.S. Pat. No. 4,684,611, plant RNA virus-mediated transformation (as described, for example in EP 0 067 553 and U.S. Pat. No. 4,407,956), liposome-mediated transformation (as described, for example in U.S. Pat. No. 4,536,475), and other methods such as those described methods for transforming certain lines of corn (e.g., U.S. Pat. No. 6,140,553; Fromm et al., 1990, Bio/Technology 8, 833-839; Gordon-Kamm et al., 1990, The Plant Cell 2, 603-618) and rice (Shimamoto et al., 1989, Nature 338, 274-276; Datta et al. 1990, Bio/Technology 8, 736-740) and the method for transforming monocots generally (PCT publication WO92/09696). The most widely used transformation method for dicot species is Agrobacterium mediated transformation. For cotton transformation see also WO 00/71733. Brassica species (e.g. cabbage species, broccoli, cauliflower, rapeseed etc.) can for example be transformed as described in U.S. Pat. No. 5,750,871 and legume species as described in U.S. Pat. No. 5,565,346. Musa species (e.g. banana) may be transformed as described in U.S. Pat. No. 5,792,935. Agrobacterium-mediated transformation of strawberry is described in Plant Science, 69, 79-94 (1990). Likewise, selection and regeneration of transformed plants from transformed cells is well known in the art. Obviously, for different species and even for different varieties or cultivars of a single species, protocols are specifically adapted for regenerating transformants at high frequency.

Besides transformation of the nuclear genome, also transformation of the plastid genome, preferably chloroplast genome, is included in the invention. One advantage of plastid genome transformation is that the risk of spread of the transgene(s) can be reduced. Plastid genome transformation can be carried out as known in the art, see e.g. Sidorov V A et al. 1999, Plant J. 19: 209-216 or Lutz K A et al. 2004, Plant J. 37(6):906-13, U.S. Pat. No. 6,541,682, U.S. Pat. No. 6,515,206, U.S. Pat. No. 6,512,162 or U.S. Pat. No. 6,492,578.

The target gene nucleic acid sequence is inserted in a plant cell genome so that the inserted sense and/or antisense sequence is downstream (i.e. 3′) of, and under the control of, a promoter which can direct the expression in the plant cell. This is preferably accomplished by inserting the chimeric gene in the plant cell genome, particularly in the nuclear or plastid (e.g. chloroplast) genome.

Preferred promoters include: the strong constitutive 35S promoters or (double) enhanced 35S promoters (the “35S promoters”) of the cauliflower mosaic virus (CaMV) of isolates CM 1841 (Gardner et al., 1981, Nucleic Acids Research 9, 2871-2887), CabbB-S (Franck et al., 1980, Cell 21, 285-294) and CabbB-JI (Hull and Howell, 1987, Virology 86, 482-493); the 35S promoter described by Odell et al. (1985, Nature 313, 810-812) or in U.S. Pat. No. 5,164,316, promoters from the ubiquitin family (e.g. the maize ubiquitin promoter of Christensen et al., 1992, Plant Mol. Biol. 18, 675-689, EP 0 342 926, see also Cornejo et al. 1993, Plant Mol. Biol. 23, 567-581), the gos2 promoter (de Pater et al., 1992 Plant J. 2, 834-844), the emu promoter (Last et al., 1990, Theor. Appl. Genet. 81, 581-588), Arabidopsis actin promoters such as the promoter described by An et al. (1996, Plant J. 10, 107.), rice actin promoters such as the promoter described by Zhang et al. (1991, The Plant Cell 3, 1155-1165) and the promoter described in U.S. Pat. No. 5,641,876 or the rice actin 2 promoter as described in WO070067; promoters of the Cassaya vein mosaic virus (WO 97/48819, Verdaguer et al. 1998, Plant Mol. Biol. 37, 1055-1067), the pPLEX series of promoters from Subterranean Clover Stunt Virus (WO 96/06932, particularly the S7 promoter), a alcohol dehydrogenase promoter, e.g., pAdh1S (GenBank accession numbers X04049, X00581), and the TR1′ promoter and the TR2′ promoter (the “TR1′promoter” and “TR2′promoter”, respectively) which drive the expression of the 1′ and 2′ genes, respectively, of the T-DNA (Velten et al., 1984, EMBO J. 3, 2723-2730), the Figwort Mosaic Virus promoter described in U.S. Pat. No. 6,051,753 and in EP426641, histone gene promoters, such as the Ph4a748 promoter from Arabidopsis (PMB 8: 179-191), or others.

Alternatively, a promoter can be utilized which is not constitutive but rather is specific for one or more tissues or organs of the plant (tissue preferred/tissue specific, including developmentally regulated promoters), for example root preferred whereby the CCD or NCED gene is expressed only in cells of the specific tissue(s) or organ(s) and/or only during a certain developmental stage, for example during the early stages of plant development when the plant is most vulnerable to parasite attack.

For root specific expression a promoter preferentially active in roots is described in WO00/29566. Another promoter for root preferential expression is the ZRP promoter (and modifications thereof) as described in U.S. Pat. No. 5,633,363. Other suitable root-specific promoters are MtPT1, expressed in roots and root hairs in red clover (pers. comm. Zengyu Wang, The Samuel Roberts Noble Foundation, Ardmore, USA), pHM62, pHM58, pHM72, pHM78 (pers. comm. Hiromi Higo, Plant Functional genomics Co., Ibaraki, Japan) and Catb delt9 (pers. comm. Masao Iwamoto, National Institute of Agrobiological Sciences, Ibaraki, Japan).

Another alternative is to use a promoter whose expression is inducible. The reduction of carotenoid and/or germination stimulant formation may thus only develop after induction of target gene expression, for example upon a change in temperature, wounding, chemical treatment (e.g. substrate-inducible) etc. Examples of inducible promoters are wound-inducible promoters, such as the MPI promoter described by Cordera et al. (1994, The Plant Journal 6, 141), which is induced by wounding (such as caused by insect or physical wounding), or the COMPTII promoter (WO0056897) or the promoter described in U.S. Pat. No. 6,031,151. Alternatively the promoter may be inducible by a chemical, such as dexamethasone as described by Aoyama and Chua (1997, Plant Journal 11: 605-612) and in U.S. Pat. No. 6,063,985 or by tetracycline (TOPFREE or TOP 10 promoter, see Gatz, 1997, Annu Rev Plant Physiol Plant Mol Biol. 48: 89-108 and Love et al. 2000, Plant J. 21: 579-88). Other inducible promoters are for example inducible by a change in temperature, such as the heat shock promoter described in U.S. Pat. No. 5,447,858, by anaerobic conditions (e.g. the maize ADH1S promoter), by light (U.S. Pat. No. 6,455,760), by pathogens (e.g. EP759085 or EP309862) or by senescence (SAG12 and SAG13, see U.S. Pat. No. 5,689,042). Obviously, there are a range of other promoters available.

The target gene sense and/or antisense sequence is inserted into the plant genome so that the sense and/or antisense sequence is upstream (i.e. 5′) of suitable 3′ end transcription regulation signals (“3′end”) (i.e. transcript formation and polyadenylation signals). Polyadenylation and transcript formation signals include those of the CaMV 35S gene (“3′ 35S”), the nopaline synthase gene (“3′nos”) (Depicker et al., 1982 J. Molec. Appl. Genetics 1, 561-573.), the octopine synthase gene (“3′ocs”) (Gielen et al., 1984, EMBO J. 3, 835-845) and the T-DNA gene 7 (“3′ gene 7″) (Velten and Schell, 1985, Nucleic Acids Research 13, 6981-6998), which act as 3′-untranslated DNA sequences in transformed plant cells, and others.

Introduction of the T-DNA vector into Agrobacterium can be carried out using known methods, such as electroporation or triparental mating.

A target gene nucleic acid sequence can optionally be inserted in the plant genome as a hybrid gene sequence whereby the target gene sequence is linked in-frame to a (U.S. Pat. No. 5,254,799; Vaeck et al., 1987, Nature 328, 33-37) gene encoding a selectable or scorable marker, such as for example the neo (or nptII) gene (EP 0 242 236) encoding kanamycin resistance, so that the plant expresses a fusion protein which is easily detectable.

Preferably, for selection purposes but also for weed control options, the transgenic plants of the invention are also transformed with a DNA encoding a protein conferring resistance to herbicide, such as a broad-spectrum herbicide, for example herbicides based on glufosinate ammonium as active ingredient (e.g. Liberty® or BASTA; resistance is conferred by the PAT or bar gene; see EP 0 242 236 and EP 0 242 246) or glyphosate (e.g. RoundUp®; resistance is conferred by EPSPS genes, see e.g. EP0 508 909 and EP 0 507 698). Using herbicide resistance genes (or other genes conferring a desired phenotype) as selectable marker further has the advantage that the introduction of antibiotic resistance genes can be avoided. Alternatively, other selectable marker genes may be used, such as antibiotic resistance genes. As it is generally not accepted to retain antibiotic resistance genes in the transformed host plants, these genes can be removed again following selection of the transformants. Different technologies exist for removal of transgenes. One method to achieve removal is by flanking the chimeric gene with lox sites and, following selection, crossing the transformed plant with a CRE recombinase-expressing plant (see e.g. EP506763B1). Site specific recombination results in excision of the marker gene. Another site specific recombination systems is the FLP/FRT system described in EP686191 and U.S. Pat. No. 5,527,695. Site specific recombination systems such as CRE/LOX and FLP/FRT may also be used for gene stacking purposes. Further, one-component excision systems have been described, see e.g. WO9737012 or WO9500555).

When reference to “a transgenic plant cell” or “a recombinant plant cell” is made anywhere herein, this refers to a plant cell (or also a plant protoplast) as such in isolation or in tissue/cell culture, or to a plant cell (or protoplast) contained in a plant or in a differentiated organ or tissue, and these possibilities are specifically included herein. Hence, a reference to a plant cell in the description or claims is not meant to refer only to isolated cells in culture, but refers to any plant cell, wherever it may be located or in whatever type of plant tissue or organ it may be present. Also, parts removed from the recombinant plant, such as harvested fruit, seeds, cut flowers, pollen, etc. as well as cells derived from the recombinant cells, such as seeds derived from traditional breeding (crossing, selfing, etc.) which retain the chimeric target gene are specifically included.

### Overexpression of Target Genes and Methods for Making Catch and Trap Crops

In principle, any plant that is parasitised by Orobanche or Striga spp. or could be used as a trap or catch crop may be a suitable host for overexpression. Target genes as defined above can be overexpressed using methods as described above, with the difference that the whole coding sequence (cDNA or genomic) of one or more target genes is used to make an expression vector, which is then used in the transformation method. Thus, in the above description reference to sense and/or antisense sequences is simply replaced by sense sequences encoding the target enzyme(s). A higher production of germination stimulants can be easily assessed using for example the germination bioassay provided in the present invention (Example 1, 3 and 7). A higher production of the strigolactones may also improve mycorrhizal colonisation. Overexpression leads to (significantly) enhanced amounts of one or more germination stimulants being produced by the recombinant cells, which in turn leads to (significantly) enhanced germination of the parasitic seeds. “Significantly enhanced amount(s)” refers herein to an increase of at least 5%, 10%, 20%, 30%, 40%, 50%, 70%, 80%, 90%, 95% or 100% of one or more strigolactones being produced, than produced by the control plant/cell/tissue. The enhanced amount of strigolactone production is easily assayed by, for example, using a germination bioassay, as described in the Examples. This assay is an indirect assay which however, due to its sensitivity, provides a good indication of the amounts of germination inhibitors being produced. Thus, the percentage of germination of the parasitic seeds is preferably at least 5%, 10%, 20%, 30%, 40%, 50%, 70%, 80%, 90%, 95% or 100% higher in recombinant cells, tissues or plants, than in control plant/cell/tissue. Alternatively, biochemical or molecular assays may be used.

### Uses of the Recombinant Plants and Plant Host Cells

The resulting transformed plant can be used in a conventional plant breeding scheme to produce more transformed plants with the same characteristics or to introduce the transgene into other varieties of the same or related plant species. Seeds, which are obtained from the transformed plants, preferably contain the chimeric target gene (resulting in overexpression or silencing) as a stable genomic insert, and optionally one or more other chimeric genes as described above. Preferred host plants are crop plants, i.e. plants cultivated by humans for food, feed or ornamental purposes.

### Spontaneous and Induced Mutant Plants

As described herein above, it is also an object of the invention to provide a method for making non-recombinant plants which produce significantly reduced or significantly enhanced amounts of one or more germination stimulants. The method comprises selecting plants having one or more mutations in at least one carotenoid pathway gene (target gene) and producing significantly reduced levels or significantly enhanced levels of one or more germination stimulants. It is noted that when referring to a mutation in a gene, the mutation need not be in the coding sequence of the gene, but may for example be in the promoter region. Also, “mutation” refers to insertion, deletion and/or replacement of one or more nucleotides. The mutation may be in any gene coding for an enzyme involved in carotenoid biosynthesis or carotenoid catabolism to the strigolactone germination stimulants.

The selection may be carried out using various known methods, such as molecular or biochemical methods, followed by e.g. a germination bioassay. Optionally, selection of cells, tissues or plants may be preceded by mutagenesis, such as chemical mutagenesis (e.g. as done in TILLING), transposon mutagenesis, UV- or gamma-radiation treatment, etc.

### SEQUENCES

SEQ ID NO 1: Zea mays phytoene synthase 1 cDNA
SEQ ID NO 2: Zea mays phytoene synthase 1 protein
SEQ ID NO 3: Zea mays phytoene desaturase cDNA
SEQ ID NO 4: Zea mays phytoene desaturase protein
SEQ ID NO 5: Zea mays ξ-carotene desaturase cDNA
SEQ ID NO 6: Zea mays ξ-carotene desaturase protein
SEQ ID NO 7: Lycopersicon esculentum carotene isomerase cDNA
SEQ ID NO 8: Lycopersicon esculentum carotene isomerase protein
SEQ ID NO 9: Zea mays lycopene-β-cyclase cDNA
SEQ ID NO 10: Zea mays lycopene-β-cyclase protein
SEQ ID NO 11: Oryza sativa β-carotene hydroxylase cDNA
SEQ ID NO 12: Oryza sativa β-carotene hydroxylase protein
SEQ ID NO 13: Oryza sativa zeaxanthin epoxidase cDNA
SEQ ID NO 14: Oryza sativa zeaxanthin epoxidase protein
SEQ ID NO 15: Solanum tuberosum neoxanthin synthase cDNA
SEQ ID NO 16: Solanum tuberosum neoxanthin synthase protein
SEQ ID NO 17: Zea mays vp14=9-cis-epoxycarotenoid dioxygenase (NCED) cDNA
SEQ ID NO 18: Zea mays vp14=9-cis-epoxycarotenoid dioxygenase (NCED) protein
SEQ ID NO 19: Zea mays carotenoid cleavage dioxygenase 1 cDNA (CCD1)
SEQ ID NO 20: Zea mays carotenoid cleavage dioxygenase 1 protein (CCD1)
SEQ ID NO 21: Oryza sativa 9-cis-epoxycarotenoid dioxygenase (NCED-3) cDNA
SEQ ID NO 22: Oryza sativa 9-cis-epoxycarotenoid dioxygenase (NCED-3) protein
SEQ ID NO 23: Oryza sativa 9-cis-epoxycarotenoid dioxygenase (NCED-5) cDNA
SEQ ID NO 24: Oryza sativa 9-cis-epoxycarotenoid dioxygenase (NCED-5) protein
SEQ ID NO 25: Oryza sativa 9-cis-epoxycarotenoid dioxygenase (NCED-4) cDNA
SEQ ID NO 26: Oryza sativa 9-cis-epoxycarotenoid dioxygenase (NCED-4) protein
SEQ ID NO 27: Oryza sativa 9-cis-epoxycarotenoid dioxygenase (NCED-1) cDNA
SEQ ID NO 28: Oryza sativa 9-cis-epoxycarotenoid dioxygenase (NCED-1) protein
SEQ ID NO 29: Oryza sativa 9-cis-epoxycarotenoid dioxygenase (NCED-2) cDNA
SEQ ID NO 30: Oryza sativa 9-cis-epoxycarotenoid dioxygenase (NCED-2) protein
SEQ ID NO 31: Oryza sativa 9-cis-epoxycarotenoid dioxygenase (NCED-like) cDNA
SEQ ID NO 32: Oryza sativa 9-cis-epoxycarotenoid dioxygenase (NCED-like) protein
SEQ ID NO 33: Oryza sativa lignostilbene 1 cDNA
SEQ ID NO 34: Oryza sativa lignostilbene 1 protein
SEQ ID NO 35: Oryza sativa carotenoid cleavage dioxygenase 8 (CCD8) cDNA
SEQ ID NO 36: Oryza sativa carotenoid cleavage dioxygenase 8 (CCD8) protein
SEQ ID NO 37: Oryza sativa neoxanthin 1 cleavage enzyme cDNA
SEQ ID NO 38: Oryza sativa neoxanthin 1 cleavage enzyme protein
SEQ ID NO 39: Oryza sativa neoxanthin 2 cleavage enzyme cDNA
SEQ ID NO 40: Oryza sativa neoxanthin 2 cleavage enzyme protein
SEQ ID NO 41: Oryza sativa lignostilbene 2 cDNA
SEQ ID NO 42: Oryza sativa lignostilbene 2 protein
SEQ ID NO 43: Oryza sativa crocetin cDNA
SEQ ID NO 44: Oryza sativa crocetin protein
SEQ ID NO 45: Oryza sativa dioxygenase cDNA
SEQ ID NO 46: Oryza sativa dioxygenase protein
SEQ ID NO 47: Oryza sativa carotenoid cleavage dioxygenase 7 (CCD7) cDNA
SEQ ID NO 48: Oryza sativa carotenoid cleavage dioxygenase 7 (CCD7) protein
SEQ ID NO 49: Oryza sativa carotenoid cleavage dioxygenase 1 (CCD1) cDNA SEQ ID NO 50: Oryza sativa carotenoid cleavage dioxygenase 1 (CCD1) protein

### FIGURE LEGENDS

FIG. 1

Structure of strigolactone germination stimulants. (a) (+)-strigol, (b) orobanchol, (c) sorgolactone, (d) synthetic germination stimulant GR24. A, B, C and D indicate the A-, B-, C- and D-ring.

FIG. 2

Carotenoid and abscisic acid biosynthetic pathway. Intermediates that are not shown are in square brackets. The 15-cis and 9,9′-cis between brackets for phytoene and ξ-carotene refer to new insights in carotene isomerisation during early steps of the pathway (Park et al., 2002; Isaacson et al., 2004). Carotenoid maize mutants (italics) and inhibitors (underlined) at different steps in the pathway that are described in the present invention are indicated with [symbol] . Redrawn from (Cunningham and Gantt, 1998, Annu Rev Plant Physiol Plant Mol Biol, 49, 557-583; Hirschberg, 2001, supra) and (Seo and Koshiba, 2002, supra).

FIG. 3

Germination of S. hermonthica seeds induced by root exudates of maize (W22 or Dent, as indicated in the graphs) (A-D), cowpea (E) and sorghum (G) and germination of O. crenata induced by the root exudates of cowpea (F) as affected by treatment of the host seedlings.

A, maize: 100 μM fosmidomycin (Fos), 25 μM fluridone (Flu), 10 μM mevastatin (Me), untreated control (C). B, maize: 10 μM fluridone (Flu) applied to plants growing under normal light (Flu) and dim light (FluD), untreated controls (C and CD) C, maize: 200 μM amitrole (Ami), untreated control (C). Seedlings were grown at 21° C. D, maize: untreated control (C), 10 μM fluridone (Flu), 0.1 mM naproxen (0.1 N), 1 mM naproxen (1 N), 0.1 mM sodium tungstate (0.1 ST), 1 mM sodium tungstate (1 mM ST), 0.02 mM abscisic acid plus 10 μM fluridone (0.02 ABA Flu), 0.02 mM abscisic acid (0.02 ABA), 0.2 mM abscisic acid plus 10 μM fluridone (0.2 ABA Flu), 0.2 mM abscisic acid (0.2 ABA). E, cowpea: 10 μM fluridone, nontreated control (C), 10 μM fluridone (Flu), root exudate of fluridone treated seedlings plus 0.01 mg.L⁻¹ GR24 (Flu GR 0.01), 0.01 and 0.1 mg.L⁻¹ GR24 alone (GR 0.01 and GR 0.1). F, cowpea (with O. crenata): 10 μM fluridone (Flu), nontreated control (C). G, sorghum: 4 μM fluridone (Flu), nontreated control (C).

Within each experiment, the concentrations of root exudates were equalized, by dilution, for differences in root fresh weight. Data represent average germination±standard error of 8 to 10 individual plants and 2 or 3 disks per plant (indicated in each diagram by 8/3 etc), with about 50-100 S. hermonthica or O. crenata seeds each. Statistical analysis showed significant differences (P<0.05) for all treatments, except the treatments with 100 μM fosmidomycin, 10 μM mevastatin and 0.1 mM naproxen.

FIG. 4

A, Phenotypes of mutant y10 seedlings (white) and corresponding wild type siblings (grey) and germination of S. hermonthica induced by the root exudates of these seedlings. B, Phenotypes of mutant cl1 311AA seedlings (white) and corresponding wild type siblings (grey) and germination of S. hermonthica induced by root exudates of these seedlings. N=non-mutant phenotype, M=mutant phenotype. Bars indicate average germination±standard error of 3 disks per plant with about 50-100 S. hermonthica seeds each.

FIG. 5

Average germination of S. hermonthica induced by root exudates of maize mutants Iwl, y10, al1y3, vp5, y9, cl1 311AA and vp14 and their corresponding wild type siblings (the number of seedlings used for each bioassay are indicated between brackets). Statistical analysis showed that lw1, y10, al1y3, vp5, y9 mutant phenotype induced significantly lower (P<0.05) germination than the corresponding non-mutant phenotype seedlings. The difference in germination induced by the vp14 mutant was not significant (P=0.09). N=non-mutant phenotype, M=mutant phenotype. Bars indicate average germination±standard error of 5 to 8 individual plants (indicated below the X-axis) and 3 disks per plant with about 50-100 S. hermonthica seeds each.

FIG. 6

Postulated biogenetic scheme for the formation of strigolactones.

FIG. 7

Effect of application of the inhibitor fluridone to rice roots (A) or leafs (B) on the germination/attachment of Striga hermonthica seeds. Data presented are the mean of six independent replicates.

FIG. 8

Effect of maize root exudates of roots colonized by G. intraradices on germination of Striga hermonthica seeds as induced by root exudates collected from these plants in two independent experiments (A and B). The concentrations of all root exudates were adjusted to reflect the same weight of roots per mL of exudates. Germination percentages are averages of exudates of 3 individual plants. Average colonization percentages of roots by mycorrhiza were: A: 10% (12d); 10% (19d); 52% (26d); 63% (33d); 57% (40d) and B: 11% (14d); 30% (21d); 28% (28d); 47% (34d). GR24 (0.1/0.01 mg.L⁻¹) was used as positive control, H₂O as negative control. Error bars indicate standard error.

FIG. 9

Diagram describing the RNAi constructs that are used for transformation of maize, rice and tomato.

TABLE 1
Carotenoid composition of shoots and roots of 10-day old maize seedlings
treated with inhibitors of abscisic acid biosynthesis or abscisic
acid itself and carotenoid mutants al1y3 and cl1 311AA.

| | | | |
|---|---|---|---|
| neoxanthin | violaxanthin | lutein | β-carotene |

Shoots

| | | | | |
|---|---|---|---|---|
| control | 153.40 | 72.65 | 88.56 | 65.51 |
| 10 μM fluridone | n.d. | n.d. | n.d. | n.d. |
| 1 mM naproxen | 111.81 | 52.08 | 68.58 | 47.56 |
| 0.1 mM sodium tungstate | 147.58 | 72.19 | 83.94 | 64.29 |
| 0.02 mM abscisic acid | 115.29 | 50.84 | 73.01 | 47.35 |

Roots

| | | | | |
|---|---|---|---|---|
| control | n.d. | 0.11 | n.d. | 0.02 |
| 10 μM fluridone | n.d. | n.d. | n.d. | n.d. |
| 1 mM naproxen | n.d. | 0.29 | n.d. | 0.04 |
| 0.1 mM sodium tungstate | n.d. | 0.22 | n.d. | 0.03 |
| 0.02 mM abscisic acid | n.d. | 0.07 | n.d. | 0.01 |

Carotenoid mutants
Shoots

| | | | | |
|---|---|---|---|---|
| Al1y3 | 32.43 | 31.06 | 55.72 | 46.47 |
| Mutant al1y3 | 2.64 | 3.15 | 6.17 | 4.68 |
| Cl1 311AA | 28.06 | 26.77 | 50.95 | 36.44 |
| Mutant cl1 311AA | n.d. | 1.14 | 1.08 | n.d. |

Roots

| | | | | |
|---|---|---|---|---|
| Al1y3 | n.d. | n.d. | n.d. | n.d. |
| Mutant al1y3 | n.d. | n.d. | n.d. | n.d. |
| Cl1 311AA | n.d. | 0.07 | n.d. | 0.01 |
| Mutant cl1 311AA | n.d. | 0.08 | n.d. | 0.01 |

Carotenoid content in μg · g⁻¹ fwt; n.d. - below detection limit.

The following non-limiting Examples illustrate the different embodiments of the invention. Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, and Sambrook and Russell (2001) Molecular Cloning: A Laboratory Manual, Third Edition, Cold Spring Harbor Laboratory Press, NY; and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK.

### EXAMPLES

### Example 1

### General Material and Methods

### 1.1 Plant Material and Chemicals

Maize inbred W22 was obtained from Vicky Child, IACR, UK and inbred line Dent was obtained from J C Robinson Seeds, The Netherlands. Maize seeds deficient in chlorophyll and carotenoid biosynthesis were obtained from the Maize Genetics COOP Stock Center, Urbana, Ill. (mutants lw1, y10, vp5, vp14, y9, all-y3, cl1 311AA). Cowpea (Vigna unguiculata (L.) Walp) seeds were obtained from Seriba Katilé, Institut d'Economie Rurale, Mali. Sorghum bicolor, variety CSH-1 was obtained from Bob Vasey, Sheffield University, UK. Striga (Striga hermonthica (Del.) Benth) seeds were collected from a maize field in 1994, Kibos, Kenya and Striga hermonthica seeds used in germination bioassay with sorghum root exudates were collected from a sorghum field in Sudan in 1995 and were obtained from Bob Vasey. Orobanche crenata seeds were obtained from D. M. Joel, Newe-Ya'ar Research Center, Israel.

The following inhibitors of isoprenoid pathways were used: mevastatin (Sigma-Aldrich), fosmidomycin (Molecular Probes, Inc.), fluridone (Ducheva), amitrole (3-amino-1,2,4-triazole), naproxen (S)-6-methoxy-α-methyl-2-naphthaleneacetic acid sodium salt), sodium tungstate, dihydrate) and (±)-abscisic acid (all from Sigma-Aldrich). The synthetic germination stimulant strigolactone analogue GR24 was kindly provided by prof. B. Zwanenburg, University of Nijmegen, The Netherlands.

### 1.2 Root Exudate Collection

Seeds of maize were sterilized in 4% sodium hypochlorite containing 0.02% (v/v) Tween 20, rinsed thoroughly with sterile water and imbibed for 24 hrs on moistened filter paper at 25° C. Plants were grown in autoclaved perlite in separate tubes or in 1-L pots (ø15 cm) at 25° C. in a climate room with 16/8 hrs photoperiod at 28 μmol photons.m⁻².sec⁻¹. To grow plants under dim light, plants were grown in a box covered by several layers of cheesecloth and aluminum foil with small holes (0.5 μmol photons. m⁻².sec⁻¹). The plants were watered with tap water (control, carotenoid mutants) or with solutions of abscisic acid or inhibitors. Depending on the vigor, mutants were grown for 8-11 days. After several days, depending on the experiment, seedlings were removed from perlite and carefully cleaned from perlite. Plants were put into about 8 mL tap water in glass tubes at 25° C. to collect root exudates. Tubes were covered with aluminum foil to exclude light. After 6-24 hrs root exudates were collected and root fresh weight of each seedling was determined. Exudates were diluted to equal concentrations (mL of exudate per gram of roots) within each experiment and subsequently used in a bioassay. Seeds of cowpea were imbibed for 24 hrs on moistened filter paper at 25° C. in darkness. Imbibed seeds were sown in pots containing a mixture of vermiculite and perlite (1:1). Plants were grown in a climate room with 16/8 hrs photoperiod at 28 μmol photons.m⁻².sec⁻¹ at 25° C. The plants were regularly watered for 4 days. Subsequently, seedlings were watered with water (control) or with 10 μM fluridone for 5 days. Root exudates were collected as described for maize. Seeds of sorghum were germinated for 3 days at 28° C. in autoclaved vermiculate, watered with 40% modified Long Ashton nutrient solution (Gurney et al., 2002, Weed Res, 42, 317-324). Small seedlings were transferred to glass tubes to grow hydroponically in a phytotron 14/10 hrs photoperiod at 250 μmol photons.m⁻².sec-1 at 28/23° C. 12 days old seedlings were transferred into clean tubes, containing 40% Long Ashton nutrient solution without/with 4 μM fluridone. 5 days later root exudates were collected during 24 h as described for maize.

### 1.3 Germination Bioassay

Root exudates produced by individual plants were tested by bioassay. The seeds of Orobanche and Striga spp. require preconditioning (or warm stratification) for a certain period of time at a suitable temperature before the seeds become responsive to germination stimulants (Matusova et al., 2004, Seed Sci Res, 14, 335-344). Preconditioning was performed under sterile conditions. The seeds were surface sterilized in 2% sodium hypochlorite containing 0.02% (v/v) Tween 20 for 5 min and rinsed thoroughly with sterile demineralized water. Subsequently the seeds were dried for 30 min in a laminar air flow cabinet. Approximately 50-100 seeds were spread on a glass fiber filter paper (GFFP) disk (Ø 9 mm) and put into sterile Petri dishes (Ø 9 cm) lined with Whatman filter paper wetted with 3 mL of demineralized water. Petri dishes were sealed with parafilm and incubated for preconditioning. S. hermonthica seeds were preconditioned at 30° C. in darkness for 10-12 days, O. crenata seeds were preconditioned at 21° C. in darkness for 14 days. After the preconditioning period the GFFP disks with S. hermonthica or O. crenata seeds were removed from the Petri dish and dried for 20 min to remove surplus moisture. The disks were transferred to another Petri dish within a filter paper ring (outer Ø 9 cm, inner Ø 8 cm) wetted with 0.9 ml of H₂O, which maintained a moist environment during the germination bioassay. 50 μl of the test solutions were added to duplicate or triplicate disks, depending on the experiment. The synthetic germination stimulant GR24 (0.01 and 0.1 mg.L⁻¹ GR24) as a positive and water as a negative control were included in each bioassay. Seeds were incubated at 30° C. (S. hermonthica) or 25° C. (O. crenata) in darkness for 2 days (S. hermonthica) or 6 days (O. crenata). The germinated and non-germinated seeds were counted using a binocular. Seeds were considered germinated when the radicle protruded the seed coat. To test for an inhibitory effect of root exudates from fluridone-treated seedlings, a control combining root exudates of maize, cowpea or sorghum in combination with GR24 was included in the germination bioassays. Root exudates plus GR24 were diluted with water to the same final concentration of root exudate that was used without GR24. We used a non-saturating concentration of GR24 to ensure that any inhibitory (negative) effect of the root exudate of fluridone-treated cowpea seedlings would be visible. The Generalized Linear Mixed Model method was used for statistical analysis of all germination bioassays using Genstat, procedure IRREML (Payne and Lanne, 1993).

### Example 2

### Elucidation of Strigolactone Formation in Maize and Cowpea

### 2.1 The Use of Inhibitors Early in the Pathway

W22 seedlings were grown in perlite in separate tubes for 3 days at 25° C. Tubes were covered with aluminum foil and the plants were watered with tap water. Then solutions of inhibitors were applied and seedlings were grown in the presence of inhibitors for an additional 5 days. 10 μM mevastatin and 100 μM fosmidomycin were used to inhibit isoprenoid formation in the cytosol and plastids, respectively and 25 μM fluridone was used to block carotenoid formation. After 5 days, seedlings were taken from the perlite and any perlite clinging to the roots was carefully removed. Plants were put into tap water in glass tubes for 6 hrs at 25° C. A dilution of 70 mg root fresh weight /mL root exudate was used for the bioassay. Root exudates from 10 individual plants for each treatment were tested. For amitrole experiments, germinated seeds of maize inbred line DENT were sown in perlite in pots and grown at 21° C. for 11 days. Control plants were watered with tap water, amitrole-treated plants were watered with 200 μM amitrole. Root exudates were collected in tap water for 8 hrs. Naproxen and sodium tungstate treated maize inbred line DENT plants were sown in perlite in pots and grown at 25° C. for 10 days. Control plants were watered with tap water, naproxen-treated plants were watered with 0.1 and 1 mM naproxen. Sodium tungstate-treated plants were watered with 0.1 and 1 mM solutions. Abscisic acid treated plants were watered with 0.02 and 0.2 mM abscisic acid without/with 10 μM fluridone. Root exudates from 8 individual seedlings/treatment were collected in tap water for 18 hrs.

Root exudates of control maize seedlings always induced germination of preconditioned S. hermonthica seeds regardless of lines and cultivars used. Water alone did not induce any germination of S. hermonthica seeds. Because the plants were grown in different experiments under different conditions, the maize-induced germination of striga seeds was analyzed within each experiment separately.

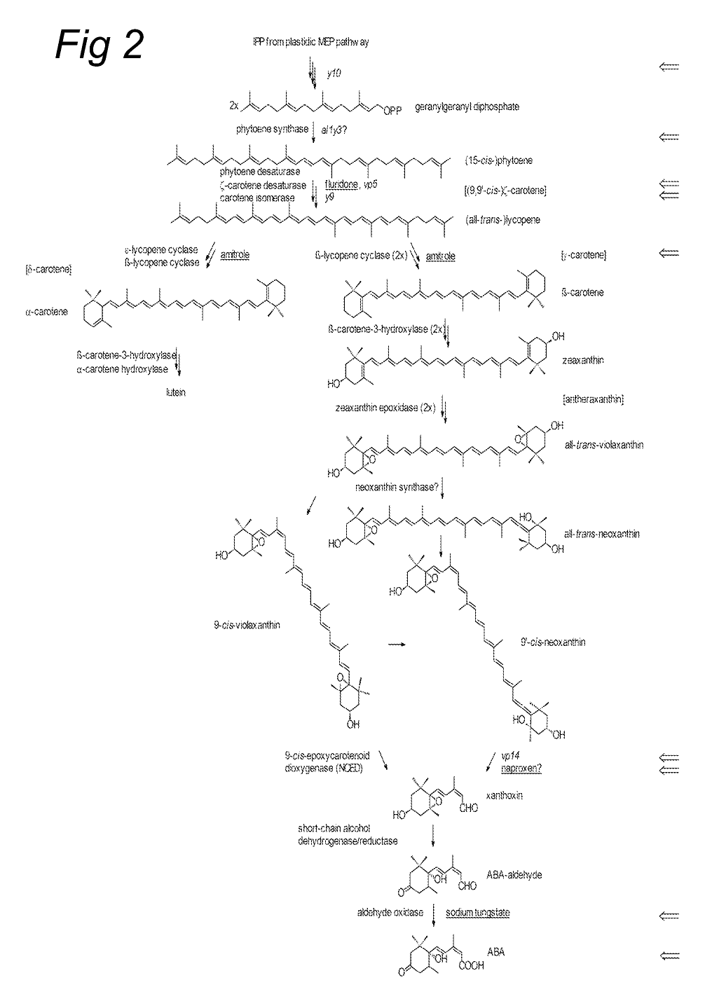

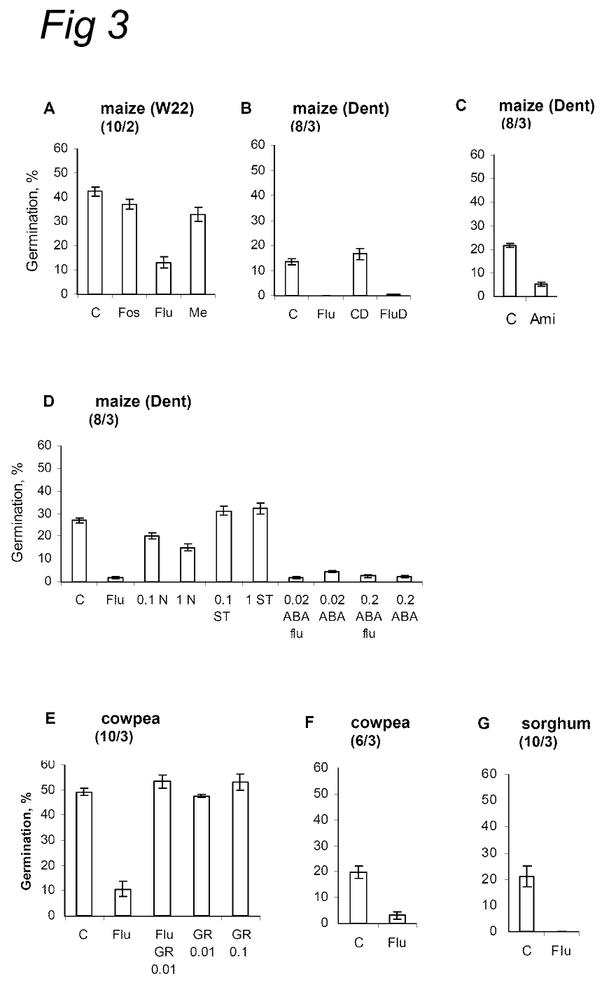

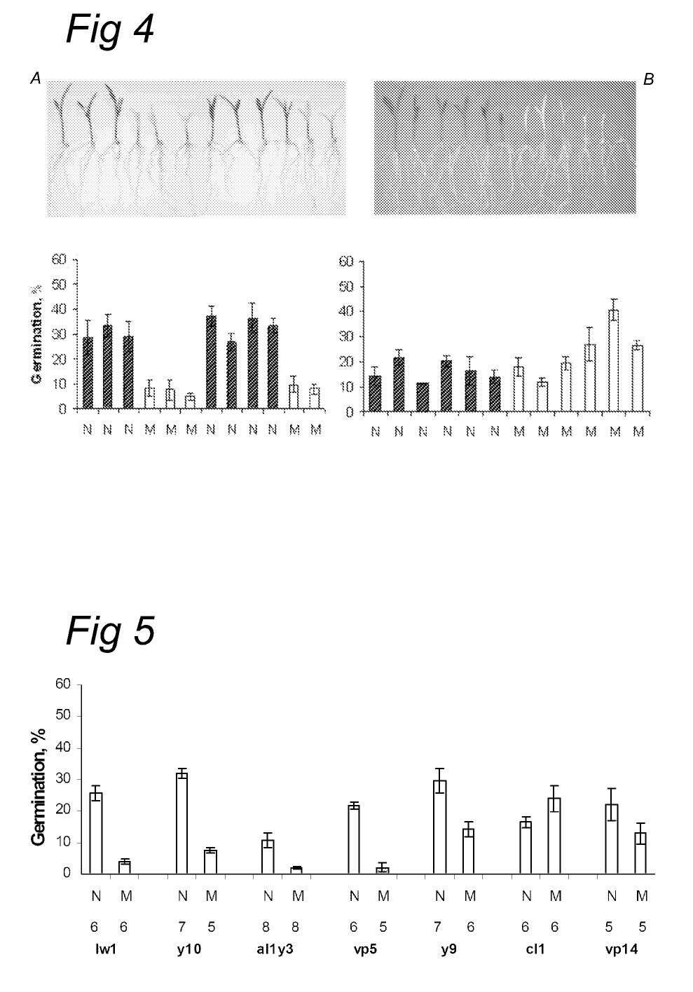

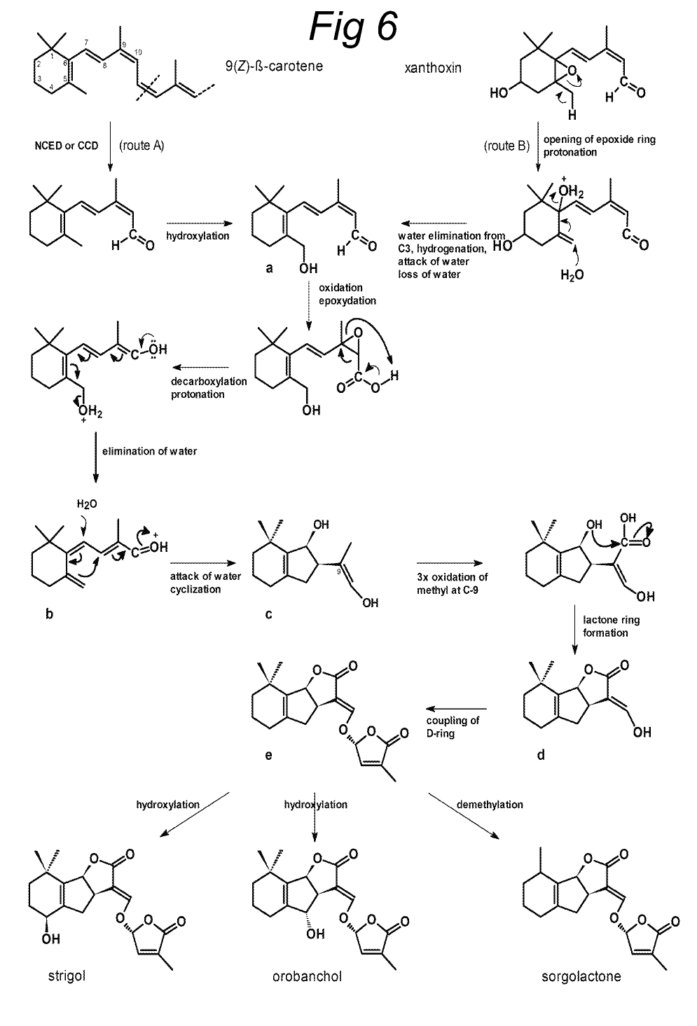

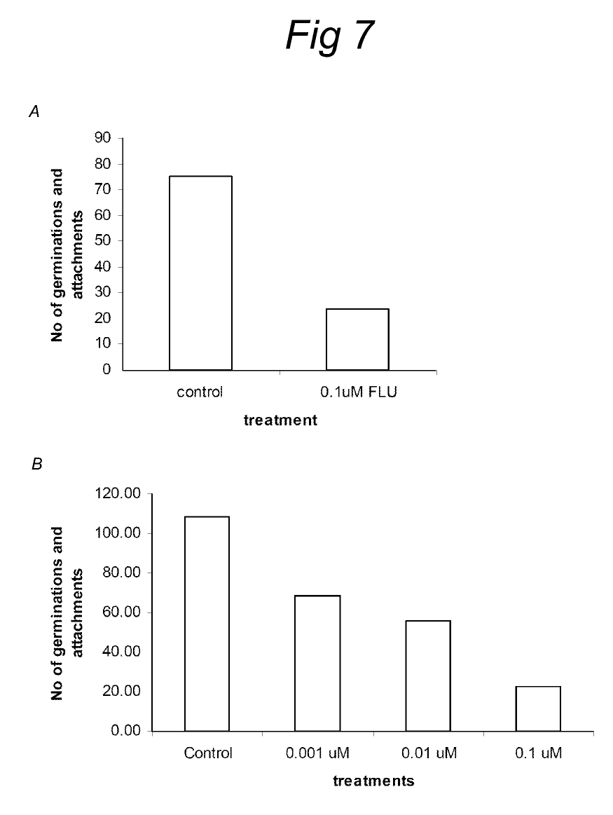

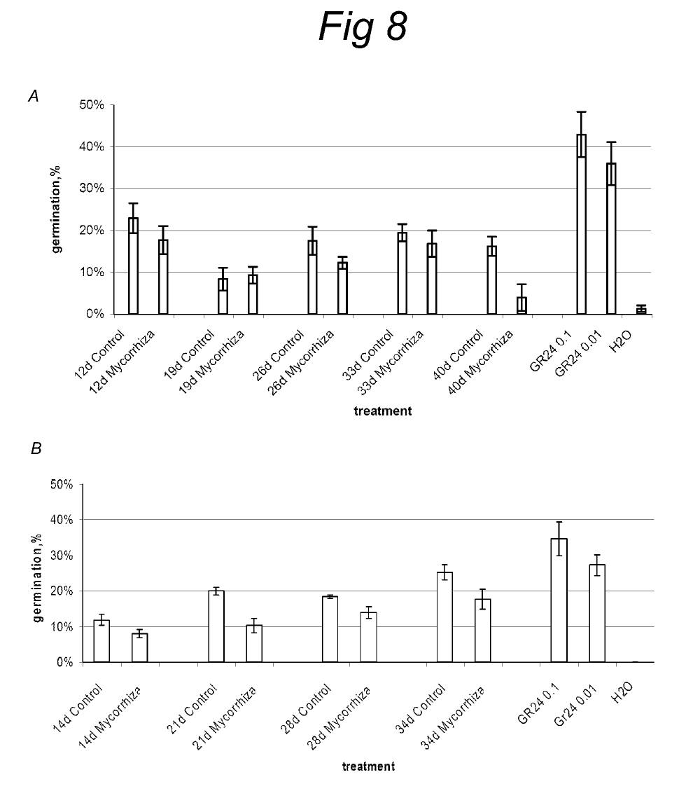

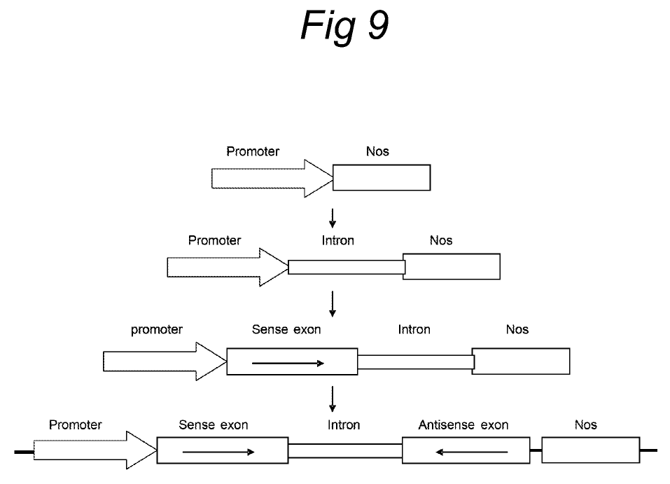

Mevastatin, an inhibitor of 3-hydroxy-3-methylglutaryl (HMG) CoA reductase, catalyzing the formation of mevalonic acid from HMG, was used to reduce cytosolic isoprenoid formation (FIG. 2). Fosmidomycin, blocking 1-deoxy-D-xylulose 5-phosphate reductoisomerase was used to inhibit plastidic isoprenoid formation and fluridone was used to block carotenoid formation in the plastids (FIG. 2). Inhibitors were applied from 3 days after germination when the root length was about 3-5 cm. Exudates from individual control plants of inbred line W22 induced a high and reproducible germination score (42±2%) (FIG. 3A). Mevastatin did not affect the morphology or appearance of the plants in contrast to fosmidomycin that induced slightly pale-yellow leaves. Mevastatin and fosmidomycin slightly but non-significantly reduced maize root exudate-induced S. hermonthica germination (37±2% for fosmidomycin and 33±3% for mevastatin-treated plants (FIG. 3A). Leaves of maize seedlings grown in the presence of the carotenoid inhibitor fluridone were white with green tips showing the proportion of newly formed tissue that was affected by fluridone treatment. Germination induced by the root exudates of fluridone-treated maize was about 80% lower than for control maize (FIG. 3A). Fluridone specifically inhibits the second dedicated enzyme in the carotenoid pathway, phytoene desaturase (PDS) (Li et al., 1996, supra) (FIG. 2). The highly reduced root exudate-induced germination of S. hermonthica by fluridone-treated plants strongly suggests that the germination stimulant produced by maize is derived from the carotenoid pathway.

Germination of S. hermonthica is also induced by cowpea root exudates and a strigolactone germination stimulant of S. gesneroides, alectrol, has been identified in cowpea root exudates (Muller et al., 1992, supra). Therefore we assayed the effect of fluridone also on cowpea. The root exudates of non-treated cowpea induced 49±2% germination of S. hermonthica seeds compared with 11±3% by fluridone-treated cowpea root exudates (FIG. 3E). To exclude the possibility that fluridone treatment leads to the formation of inhibitors of germination (rather than a lower production of the germination stimulant), the root exudates of fluridone-treated cowpea were also assessed in combinations with suboptimal concentrations of the synthetic germination stimulant GR24. The resulting germination of S. hermonthica induced by fluridone-treated cowpea root exudates with GR24 was slightly higher 53±3% than germination induced by GR24 alone (48±1%) (FIG. 3E). These results show that the germination stimulant exuded from the roots of cowpea is also derived from the carotenoid pathway and that lower germination induced by fluridone-treated root exudates is caused by a lower concentration of germination stimulants in the root exudate and not by the production of inhibitors in fluridone-treated plants. In addition to S. hermonthica, cowpea root exudates also induced the germination of seeds of O. crenata (FIG. 3F). Just like for S. hermonthica, germination of O. crenata seeds with fluridone-treated cowpea root exudate was lower than that induced by the control (FIG. 3F) showing that also for O. crenata the germination stimulant(s) in cowpea root exudate is (are) carotenoid-derived.

Fluridone, through the inhibition of carotenoid formation in the leaves, also caused photodestruction of the chlorophyll. Therefore, in a next experiment, maize seedlings were grown under dim light in order to prevent photodestruction of chlorophyll while carotenoid biosynthesis was inhibited. Control plants grown under normal light were green and root exudates induced germination of S. hermonthica seeds (13±1%) (FIG. 3B). Fluridone-treated plants exhibited the typical white phenotype and induced virtually no germination. Control seedlings grown under dim light were pale-green but they induced similar germination of S. hermonthica seeds as control seedlings grown under normal light conditions. Under dim light conditions, fluridone-treated seedlings did not show the typical bleaching visible under normal light but exhibited a pale-green phenotype similar to control seedlings under dim light. Nevertheless germination of S. hermonthica seeds induced by root exudates of fluridone-treated seedlings was also negligible (FIG. 3B). This demonstrates that the degradation/absence of chlorophyll itself is not causing the reduced germination induced by root exudates of maize in which carotenoid formation is disturbed. An additional possibility to discriminate between the direct effect of the absence of carotenoids and the indirect effect through destruction of chlorophyll is the use of chlorophyll mutants. The maize mutant cl1 (chlorophyll) (311AA) exhibits an albino phenotype and was first described by Everett (see Robertson, 1966) to be a chlorophyll mutant. Later, Robertson et al. (1966) described a group of cl1 mutants as mutants that are impaired in both chlorophyll as well as carotenoid formation to different levels depending on a clm (gene modifier) (Robertson et al., 1966, Photochem Photobiol, 5, 797-805). Therefore we analyzed both the induction of germination by the c/1311AA mutant as well as its carotenoid content. For carotenoid analysis, the shoots and roots of maize seedlings were frozen in liquid nitrogen and ground to a fine powder. Carotenoids were extracted from 1.5 g of roots and 0.5 g of shoots essentially as described before (Bino et al., New Phytologist, in press). HPLC analysis was performed according to (Fraser et al., 2000, Plant J, 24, 551-558). Identification of carotenoids was based on retention time and spectral characteristics of the standards.

Mutant phenotype siblings contained strongly reduced carotenoid levels in the shoots (Table 1). In the roots, there was no difference in the concentrations of violaxanthin and β-carotene between wildtype and mutant phenotype siblings (Table 1). The root exudate of c/1311AA seedlings induced normal germination, comparable to the wild-type phenotype seedlings (FIG. 4B).

An additional interesting inhibitor of carotenoid biosynthesis is the bleaching herbicide amitrole that inhibits carotenoid biosynthesis by blocking lycopene cyclase in maize seedlings (FIG. 2) (Dalla Vecchia et al., 2001, supra). Amitrole is only effective at a relatively low temperature (Dalla Vecchia et al., 2001, supra). When grown at 21° C., amitrole-treated inbred Dent plants had a yellow-green phenotype. Root exudates of amitrole-treated seedlings induced lower germination of S. hermonthica seeds than control seedlings that were also grown at 21° C. (FIG. 3C).

### 2.2 Using Maize Mutants

To further investigate the germination stimulant biosynthetic pathway, we tested several other maize carotenoid mutants and inhibitors for their effect on induction of S. hermonthica seed germination (FIG. 2). From the Maize Genetic COOP Stock Center we obtained a number of mutants that are characterized by a change in the accumulation of carotenoids.
The mutation causing the reduced carotenoid level in the lw1 (lemon white) carotenoid mutant is not defined yet. Seedlings of lw1 exhibit an albino phenotype. Root exudates collected from lw1 albino seedlings induced significantly lower germination of S. hermonthica in comparison to corresponding seedlings with non-mutant phenotype (FIG. 5).
Carotenoid mutant y10 is characterised by a pale-yellow endosperm color and albino seedlings. Although the exact position of the mutation in the carotenoid pathway has not been clarified, the defect in the y10 mutant is supposed to affect a step in the isoprenoid pathway preceding the biosynthesis of geranylgeranyl diphosphate (FIG. 2), which results in a reduced content of carotenoids in the endosperm and carotenoids and chlorophylls in the leaves of the mutant seedlings (Janick-Buckner et al., 1999, J Hered, 90, 507-513). Root exudates of y10 mutant seedlings induced a significantly lower germination than wild-type sibling plants (FIGS. 4,5).
The al1-y3 mutant (variation of all albescent plant 1) seedlings have a white to pale-green phenotype. According to Singh et al (Singh et al., 2003, supra) the mutation is associated with β-carotene-3-hydroxylase, but according to (Wurtzel, Recent Advances in Phytochemistry, vol. 38, in press) it is associated with phytoene synthase (FIG. 2). The root exudates of mutant al1y3 seedlings induced lower germination of S. hermonthica seeds than the sibling seedlings from a segregating ear that exhibit a non-mutant phenotype (FIG. 5). Carotenoid analysis showed 10-fold lower concentration of all detected carotenoids in the shoots of mutant seedlings than in seedlings with non-mutant phenotype (Table 1). Since also lutein content is strongly reduced in the mutant phenotype it is highly likely that al1y3 is indeed a mutant associated with phytoene synthase. In roots of mutant as well as wildtype phenotype seedlings, carotenoids were all below the detection level (Table 1).

Mutant vp5 (viviparous 5) is known to be associated with phytoene C-12,13 desaturation (FIG. 2), which causes an accumulation of phytoene (Li et al., 1996, supra). Vp5 mutant seedlings are white and root-exudate induced S. hermonthica germination was much lower than for wild-type seedlings (FIG. 5).
Mutant y9 (pale yellow9) exhibits a slightly pale-green phenotype. Y9 is associated with the conversion of ξ-carotene to lycopene in the maize endosperm (FIG. 2). Chlorophyll and carotenoids could still be detected in pale-green leaves of homozygous y9 plants, albeit in reduced amounts (Janick-Buckner et al., 2001, Maydica, 46, 41-46). Although y9 mutant seedlings also induced significantly lower germination of S. hermonthica seeds than seedlings with non-mutant phenotype, germination was higher than for the albino mutants, lw1, y10 and vp5, described above showing that the mutation in y9 indeed is not 100% effective (FIG. 5). Finally, maize mutant vp14-2274 was assessed. Vp14 is a mutation in 9-cis-epoxycarotenoid dioxygenase (NCED), an important step in the abscisic acid biosynthetic pathway (Schwartz, S. H. et al., 1997, Plant Physiol., 114, 798-798) (FIG. 2). The mutation is slightly leaky. Vp14 does not have an albino phenotype, suggesting that carotenoid formation is not affected by the mutation. Nevertheless, the mutant induced lower germination than the control (P=0.09) (FIG. 5).

### 2.3 Elucidation of Later Steps

To assess whether abscisic acid is a precursor of the germination stimulants, maize seedlings were grown in the absence or presence of fluridone in combination with 0.02 or 0.2 mM (±)-abscisic acid. The roots of abscisic acid treated plants were slightly thicker and more brittle than in non-treated seedlings and the shoots were green. Independent of the presence of fluridone, plants supplied with abscisic acid induced very low germination of S. hermonthica (FIG. 3D). Subsequently, the effects of naproxen, a putative inhibitor of NCED (Lee and Milborrow, 1997, Aust J Plant Physiol, 24, 715-726) and sodium tungstate, an inhibitor blocking oxidation of the C-1 aldehyde group of xanthoxin (Lee and Milborrow, 1997, supra) were assessed (FIG. 2). Plants treated with naproxen had a normal green phenotype with slight inhibition of root growth at the highest concentration. Naproxen reduced germination of S. hermonthica seeds to 20±1% (0.1 mM) or 15±2% (1 mM), which represents a 44% reduction compared with non-treated plants (27±2%) (FIG. 3D). Sodium tungstate used in concentrations of 0.1 mM and imM (FIGS. 4m,n) did not affect germination (FIG. 3D) even though 1 mM sodium tungstate did affect maize root morphology. Roots were much shorter, thicker and more fragile than in control seedlings.

Plants treated with fluridone, naproxen, sodium tungstate and abscisic acid were analysed for carotenoid contents of roots and shoots. In fluridone treated roots phytoene accumulated but other carotenoids could not be detected (Table 1). In naproxen treated roots the concentration of violaxanthin was about 3-fold and of β-carotene 2-fold higher than in control roots (Table 1). In sodium tungstate treated roots the amount of violaxanthin was 2-fold higher than in control plants. In contrast, in abscisic acid treated roots violaxanthin and β-carotene concentrations were 37 and 50% lower than in control roots, respectively. Also in the shoot of fluridone treated plants phytoene accumulated whereas other carotenoids were below detection level (Table 1). Sodium tungstate did not affect carotenoid accumulation in shoots. Both naproxen and abscisic acid decreased accumulation of β-carotene, violaxanthin, neoxanthin and lutein (Table 1).

### 2.4 Conclusions

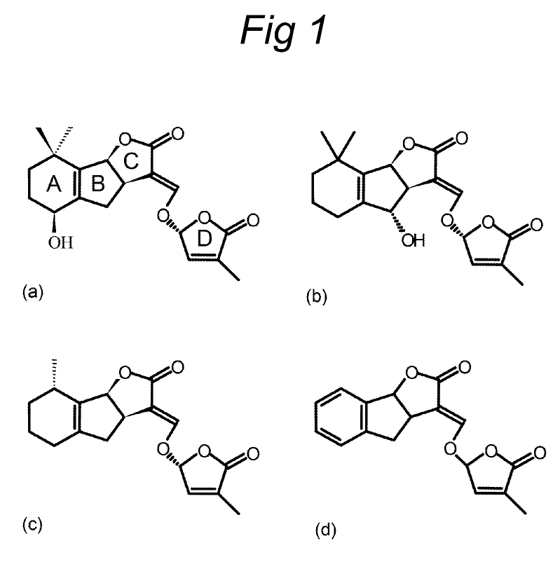

The results demonstrate that the germination stimulants of S. hermonthica present in the root exudates of maize, cowpea and sorghum are derived from the carotenoid biosynthetic pathway. We showed that this also holds for the germination stimulant(s) of O. crenata in the root exudate of cowpea. For these three host species—maize, cowpea and sorghum—the germination stimulants have been isolated and identified to be strigolactones, viz. strigol, alectrol and sorgolactone (FIG. 1) (Hauck et al., 1992, supra; Muller et al., 1992, supra; Siame et al., 1993, supra). Our results with O. crenata suggest that this species also responds to a strigolactone germination stimulant. O. crenata is not a parasite of cowpea but parasitizes legumes in more-temperate climates such as North-Africa and Spain. The germination stimulant(s) of O. crenata has not been identified yet. Root exudates of cowpea readily induced germination of O. crenata and fluridone treatment decreased this germination by about the same percentage as for S. hermonthica. This makes it likely that the germination stimulants of Orobanche spp. that parasitise legumes in temperate regions of the world are also strigolactones and are hence also derived from the carotenoid pathway. This hypothesis is supported by the detection of orobanchol, alectrol and a third unidentified strigolactone in the legume red clover (Yokota et al., 1998, supra) and alectrol in cowpea (Muller et al., 1992, supra). The fact that we demonstrate the carotenoid-origin of the germination stimulants for two parasitic plant species and three mono- and dicotyledonous hosts, and the (tentative) identification of strigolactones in the root exudates of other plant species such as red clover and tomato (Yokota et al., 1998, supra; Yoneyama et al., 2004, supra) suggest that carotenoid-derived germination stimulant formation occurs throughout the plant kingdom. The finding that sorghum root exudate induced S. hermonthica germination is completely blocked by fluridone sheds an interesting light on the discussion about the “true” nature of the sorghum germination stimulant. Lynn and coworkers have claimed that sorgoleone is the natural sorghum germination stimulant but this was disputed by Zwanenburg and coworkers based a.o. on the low water solubility of dihydrosorgoleone (Butler, 1995, supra; Keyes et al., 2001, supra; Wigchert and Zwanenburg, 1999, supra). The present results make it very likely that indeed the natural sorghum germination stimulant is sorgolactone.

The results of the present bioassays with inhibitors and mutants suggest that the biosynthesis of germination stimulants branches of from the carotenoid pathway at an intermediate that is a product of NCED action. This may be xanthoxin but could more likely be an analogue derived from cleavage of other substrates such as 9-cis-β-carotene. A biogenetic scheme can be postulated starting from such a 9-cis-β-carotene cleavage products, but also from xanthoxin (FIG. 6). However, downstream derivatives of xanthoxin or alternative carotenoids could also serve as substrate in this biogenetic scheme.

Starting from 9-cis-β-carotene, C11-12 cleavage by a dioxygenase leads to a C15-aldehyde, which upon hydroxylation yields intermediate a (FIG. 6). Alternatively, xanthoxin, upon opening of the epoxyde ring followed by protonation, elimination of water at C3 followed by hydrogenation, attack of water and loss of water could also lead to intermediate a. Oxidation followed by epoxydation and decarboxylation, protonation and elimination of water leads to intermediate b. This intermediate, upon attack of water at C7, cyclizes to intermediate c or its tautomeric aldehyde. After triple oxidation of the methyl at C9, a lactone ring will be formed with the C7 hydroxyl group to produce intermediate d. Alternatively, the methyl at C9 could already be oxidized to form a carboxyl group in compound a (not shown in FIG. 6). Instead of attack of water at C7, that carboxyl group could then attack the carbocation at C7 leading directly to intermediate d. As an alternative to lactone ring formation as described above, intermediate c could undergo keto-enol tautomerization, followed by oxidation to form a carboxyl group at C10, which can then form a lactone ring with the hydroxy at C7 (not shown in FIG. 6). Double oxidation of the methyl at C9, would then also lead to intermediate d. From intermediate d, allylic hydroxylation in ring A or B or demethylation in ring A and coupling of the D-ring will lead to strigol, orobanchol and sorgolactone, respectively (FIG. 6). The structure of alectrol is still under debate. It is not unlikely that our biogenetic scheme may help to postulate a new structure for alectrol which should subsequently be proven using chemical synthesis. Of course the order of the reactions as we propose it may in vivo be different. In addition, it is not unlikely that the D-ring is coupled to for example intermediate d before hydroxylation/demethylation to form the three different germination stimulants.

In conclusion, our results with mutants and inhibitors, and the postulated biogenetic scheme lead to the conclusion that a carotenoid cleavage dioxygenase, most likely NCED, is involved in the biosynthesis of the strigolactone germination stimulants in hosts of Orobanche and Striga spp. After this cleavage step, a number of other enzymatic reactions, such as hydroxylation, epoxydation, oxidation, etc are involved in the further modification of the primary apocarotenoid skeleton to the different strigolactones.

### Example 3

### Use of Inhibitors to Decrease Parasitic Weed Infestation

In the above examples, inhibitors of carotenoid biosynthesis were used to proof the biosynthetic origin of the germination stimulants. In this example, we describe the use of these inhibitors to reduce the formation of germination stimulants in situ, in planta and the consequences this has for parasitic weed infestation. Hereto, rice seeds (Taichung Native 1, Tn 1) were surface sterilized with 70% ethanol for 1 minute, and then washed immediately with demi water. Subsequently, 2% sodium hypochlorite plus 0.02% Tween20 were added. After 30 minutes the seeds were washed 5 times during 10 minutes in sterile demi water. The seeds were sown in a 9 cm petridish with filter paper wetted with 4 ml sterile demi water and incubated at 28° C. for two days. Then the seeds were placed in washed silver sand in pots (24 cm high×10 cm diameter). Two seeds were sown in each pot and seedlings were thinned to one per pot 7 days after sowing. Treatments with fluridone started two weeks after sowing. Fluridone was either applied directly into the sand (irrigation with 100 mL of 0.1 μM in nutrient solution) or sprayed to the leaves (sprayed until run-off with 0.001, 0.01 and 0.1 μM fluridone in water with 0.01% Tween-20). Irrigation was applied once, spraying was repeated during 3 consecutive days. Five days later, preconditioned Striga hermonthica seeds (35 mg) were applied to the roots of the rice plants. Hereto, the sand was carefully removed from the roots of the seedlings and then the Striga seeds (dispersed in water) were applied onto the root system using a syringe. Subsequently, the roots were covered with sand. Control plants were treated similarly but without fluridone treatments. After that, the pots were placed in a controlled environment greenhouse (12 hr photoperiod, 28° C. day/night, additional light and relative humidity 85%). Five replicates were used in each experiment. Throughout the experiment pots were irrigated with 40% full strength Long Ashton solution containing 20% of the normal nitrogen rate. After 6 weeks, the sand was carefully washed from the roots and the number of germinated and/or attached Striga seeds/tubercles determined using a binocular.

Irrigation with 0.1 μM of fluridone significantly reduced the number of germinated/attached Striga seeds (FIG. 7A). In control experiments, where the synthetic germination stimulant GR24 was used to induce full germination, we could conclude that fluridone was not affecting the attachment phase (but just germination) (data not shown). In the subsequent experiment fluridone was applied by spraying, and also here a strong, significant reduction in the number of germinated/attached seeds/tubercles of Striga was obtained even at 0.001 and 0.01 μM (FIG. 7B). In both experiments, the concentrations of fluridone used were so low that bleaching of the leaves did not occur.

### Example 4

### The Effect of Mycorrhizae on Germination Stimulant Formation

To investigate the effect of mycorrhizae on strigolactone production, we inoculated maize plants with Glomus intaradices, evaluated mycorrhizal colonisation at different time points after inoculation and collected exudates that we assayed for germination stimulant content using a bioassay with Striga hermonthica seeds. To ensure synchronous mycorrhization of the roots, the maize plants were grown in pots filled with 70% (v/v) expanded clay mixed with 30% (v/v) expanded clay containing Glomus intraradices Schenck & Smith inoculum/propagules in which leek had been growing, obtained from Michael Walter and Thomas Fester (Fester et al., 2002a, supra). The plants were watered with 0.5 strength Hoagland's nutrient solution with reduced phosphate content (10% of phosphate in a full strength nutrient solution). Starting after 12 days of growth under controlled conditions in a greenhouse, the maize seedlings were harvested weekly. The root and shoot weight were recorded. The level of colonization of the roots was determined by tryphan blue staining. In roots, hyphae, vesicles and arbuscules were identified. After 33 days a yellow colour was seen on the roots that were colonized by mycorrhizal fungi. This colour probably indicates the presence of mycorradicin. Root exudates were collected and germination bioassays carried out as described in Example 1 to determine the induction of Striga hermonthica germination. In both experiments mycorrhizal root exudates consistently induced lower germination than the exudates of control roots and the effect was particularly large in the first experiment 40 days after inoculation (FIG. 8).

### Example 5

### Cloning of Target Genes Involved in Strigolactone Formation

### 5.1 Introduction

Because the biosynthetic origin of the germination stimulants has now been elucidated herein, it is possible to design strategies to reduce or increase the production of germination stimulants, so crop varieties can be developed with resistance against Striga and Orobanche spp, or with improved trap/catch crop performance. Target genes for such a strategy are all the genes involved in carotenoid metabolism, carotenoid cleavage and modification of that cleavage product to the germination stimulant. The proof that this works comes from our inhibitor and mutant studies described in Example 2. In these studies the down-regulation of germination stimulant formation was obtained in a rather crude way: carotenoid biosynthesis was blocked relatively early in the pathway and throughout the plant which in most cases led to a letal or in any case growth-retarded phenotype. Nevertheless, the genes/enzymes affected in these mutants and/or by our inhibitors are suitable candidates to target, provided that the regulation is subtle. Possibilities to achieve this are the use of specific promoters, active only in the (root) tissues involved in germination stimulant formation and/or only during a limited developmental time span and/or promoters of which activity can be induced using the application of external signals, such as chemicals.

In addition to target genes/enzymes involved in the primary carotenoid/ABA pathway, such as phytoene synthase, phytoene desaturase, ξ-carotene desaturase, carotene isomerase, lycopene cyclase, β-carotene hydroxylase, zeaxanthin epoxidase and neoxanthin synthase, enzymes involved in the branching and/or the further modifications leading to the germination stimulants are suitable targets for engineering, breeding or selection for reduced or increased germination stimulant formation too. These enzymes include CCDs, NCEDs, cytochrome P450 hydroxylases and epoxidases, dehydrogenaes, demethylase, a D-ring transferase etc. To clone these genes we follow several strategies. These include subtractive techniques and transcriptomics approaches to identify genes that are downregulated by mycorrhizal colonisation, as in the present invention we show that mycorrhizal colonisation reduces strigolactone formation (Example 4). In addition, we exploit the negative effect of phosphate on orobanchol production (Koichi Yoneyama, personal communication) in subtractive and transcriptomics approaches to pinpoint genes that are down-regulated by high phosphate and/or up-regulated by low phosphate and that fit in our postulated biosynthetic scheme. In another strategy, we are cloning the first cytochrome P450 encoding gene from this pathway for example using the expected homology expected to exist with MAX1, a cytochrome P450 from Arabidopsis and oxidising the carotenoid cleavage product of a CCD (CCD7/MAX3) (Booker et al., 2005, Developmental Cell 8, 443-449; Booker et al., 2004, Current Biology 14, 1232-1238).

All the target genes/enzymes involved can be used for a transgenic approach but can also be used as markers for selection of low or high producers.

As an example, below the isolation and characterisation of a CCD and NCED from maize is described and their use to make transgenic plants with decreased germination stimulant formation. In other examples below we describe how we use rice to screen all possible carotenoid cleaving enzymes and tomato for fast evaluation.

### 5.2 Cloning of Target Genes

RNA Isolation and cDNA Synthesis

RNA from roots of maize cultivar Dent MBS847 was extracted by the SV total RNA Isolation kit from Promega, according to the manufacturer's instructions. 1 ug of total RNA was used in a volume of 3 ul, and mixed with 1 ul polyT primer (10 uM). The mixture was incubated at 70° C. for 2 minutes, and immediately put on ice for 2 minutes. Then 2 ul 5×1st strand buffer (Invitrogen), 1 ul 100 mM DTT, 1 ul 10 mM dNTP, 1 ul Rnasin (Invitrogen) and 1 ul SST Reverse Transcriptase (Invitrogen) were added and the mixture was incubated at 42° C. for 90 minutes. After this, the mixture was inactivated for 7 minutes at 70°, and stored on ice.

Amplification of cDNAs

cDNAs of target genes can be amplified using the sequences available in the databases (see e.g. sequence ID 1-18) and/or using the high homology between these sequences and genes encoding the same protein in different plant species that has been shown to be present. As an example we describe the cloning of a carotenoid cleavage dioxygenase (ZmCCD1) and a 9-cis-epoxycarotenoid dioxygenase (ZmNCED=vp14) from maize. To clone the CCD, we used a degenerate primer PCR approach. Degenerate primers CCDfwd1 (5′-TGYYTNAAYGGNGARTTYGTNMGNGTNGGNCCNAAYCCNAARTTY-3′) and CCDfwd2 (5′-GTNGCNGGNTAYCAYTGGTTYGAYGGNGAYGGNATGATHCAYGGN-3′) and CCDrev1 (5′-ATGATGCAYGAYTTYGCNATHACNGAR-3′) and CCDrev2 (5′-ACNAARAARGCNMGNTTYGGNGTNYTNCCNMGNTAYGCN-3′) were used to amplify a PCR fragment using 1 ul of cDNA in an amplification reaction mix. The mix further contained 0.5 mM dNTP, 2.5 ul 10× BD Advantage 2 PCR buffer (BD Bioscience), 0.5 ul 50× Advantage 2 polymerase mix (BD Bioscience) and 0.4 uM of forward and reverse primers. The amplification reaction mix was incubated for 5 minutes at 94°, and subsequently subjected to 30 cycles of 30 seconds 94°, 30 seconds 45° C. and 3 minutes 72° C. After these cycles, the mixture was incubated at 72° C. for 5 minutes, after which it was cooled to 10° C. 1 ul of this reaction was used as a template for a second PCR reaction, under the same conditions, but with oligonucleotides CCDfwd2 and CCDrev2. The resulting PCR fragment was cloned, sequenced and BLASTed and found to belong to accession TC220599 in the TIGR database. The full length cDNA was amplified using specific primers ZmCCD1fwd1 (5′-CTTCGCTACAAGTCATCTCG-3′) and ZmCCD1fwd2 (5′-CAAGTCATCTCGCCGCAACC-3′) and ZmCCD1rev1 (5′-AGTGAAGATACGGCACCTGC-3′) and ZmCCD1rev2 (5′-GCAGGACGTGTATTCGAACC-3′) and using the conditions as above except that for the annealing temperature 55° C. was used. The 1928 bp product was cloned into pGEMTeasy and this plasmid was sequenced. The sequence of the ZmCCD1 coding region, and the encoded protein, are provided in the Sequence information section (Sequence ID 19-20).

The ZmNCED (vp14) gene was amplified essentially as described above for ZmCCD1 but using sequence information from Genbank and specific and degenerate primers ZmNCEDfwd1 (5′-TTYGAYGGIGAYGGIATGRTICAYG-C3′), ZmNCEDfwd2 (5′-CCIAARSCIATHGGIGARYTICAYGGNCA-3′) and ZmNCEDfwd3 (5′-GARAAYTTYGTIGTIRTICCIGAYCANCA-3′) and ZmNCEDrev1 (5′-TCIGTDATIGCRAARTCRTGIATCATNGT-3′), ZmNCEDrev2 (5′-TCCCAIGCRTTCCAIARRTGRAARCARAA-3′) and ZmNCEDrev3 (5′-AYRAAIGTICCRTGRAAICCRWAIGGNAC-3′). The obtained sequence is displayed as Sequence ID 17-18.

Cloning into an Expression Vector

One ug of plasmid DNA from pGEMT-ZmCCD1 and from pRSETA was digested with BamHI and EcoRI in the appropriate buffer. Both digestions were loaded on a 1% agarose gel. After electrophoresis, fragments of the expected size (about 1650 bp for the ZmCCD fragment and about 2900 bp for the vector DNA) were observed, and isolated from the gel using Qiaex II DNA isolation kit (Qiagen). Fragments were suspended into 30 ul EB buffer (50 mM Tris pH=8.5). To clone the ZmCCDgene into pRSETA, 1 ul of BamHI-EcoRI cleaved pRSETA and 10 ul of purified and cleaved ZmCCD product were mixed with 3 ul 5xligase buffer (Invitrogen) and 1 ul of T4 ligase (Invitrogen). The ligation mixture was incubated for 3 hours at 16° C. and 10 ul was used for transformation of competent E. coli XL-1 blue by standard procedures. The transformation mixture was plated on 25 ml petridishes containing LB medium, 1.5% technical agar and 100 ug/ml ampicillin. After overnight incubation at 37° C., colonies were picked into 3 ml liquid LB medium with 100 ug/ml ampicillin and grown overnight at 37° C. shaking at 250 rpm. Plasmid was isolated from 1.5 ml of this culture using the Qiagen plasmid isolation kit, and clones containing plasmids with inserts were identified by restriction digestion with KpnI and BamHI. Plasmid pRSETA-ZmCCD1 was identified in this way. Other genes are cloned essentially similar.

### Characterisation of Cloned Genes

To assess the substrate specificity of the cloned cleavage enzymes they were either cloned into a host cell producing T7 polymerase and the carotenoid substrates to be assayed or isolated from the expression host described above and assayed in vitro by applying the relevant carotenoid substrates. For cloning into a carotenoid-producing host cell, the T7 polymerase starts transcription from the T7 promoter, which is located just upstream of the ZmCCD cDNA in plasmid pRSETA-ZmCCD#1. E. coli strain BL21 is able to produce T7 polymerase in the absence of glucose. When this strain carried plasmid pRSETA-ZmCCD1, the ZmCCD protein was produced. β-Carotene can be produced in E. coli by providing it with the plasmid pAC-BETA, which has been described by Cunningham et al. (1996; Plant Cell 8, 1613-1626). To construct bacteria that are capable of producing both β-carotene and ZmCCD or NCED enzyme, E. coli BL21 CodonPlus-RIL (Stratagene) competent cells were transformed with pAC-BETA according to the manufacturer's instructions. Recombinant E. coli were selected overnight at 37° C. on LB-agar plates with 1% glucose and 30 ug/ml chloramphenicol. A colony of E. coli BL21 with pAC-BETA was inoculated in 1 ml LB with chloramphenicol and glucose and the culture grown overnight at 250 rpm and 37°. The BL21-pAC-BETA was made competent by diluting the overnight culture 100-fold in fresh LB medium with 1% glucose, and shaking it at 37° C. until an optical density at 600 nm of 0.4 was reached. 10 MI of culture was centrifuged for 5 minutes at 400×g. Supernatant was discarded and replaced by 10 ml of an ice-cold solution of 10 mM CaCl2 and 1 mM Tris-HCl pH=7.5. Cells were resuspended and immediately centrifuged again at 400×g for 5 minutes. After discarding the supernatant, cells were resuspended in 2 ml of an ice-cold solution of 75 mM CaCl2 and 1 mM Tris-HCl pH=7.5. After incubation on ice for at least 30 minutes, cells were used for plasmid transformation by standard procedures. Plasmids pRSETA and pRSETA-ZmCCD1 were used to transform these cells, and transformed colonies were selected on LB-agar plates supplied with 1% glucose, 20 ug/ml chloramphenicol and 50 ug/ml ampicillin. β-Carotene cleavage by ZmCCD was detected by discoloration of β-carotene in BL21-pAC-BETA-pRSETA-ZmCCD colonies, zeaxanthin cleavage by discoloration of zeaxanthin in BL21-pAC-ZEA-pRSETA-ZmCCD colonies and lycopene cleavage by discoloration of lycopene in BL21-pAC-ZEA-pRSETA-ZmCCD colonies. Alternative substrates are tested in vitro. Hereto, in vitro enzyme assays are used as described in the literature (e.g. (Bouvier et al., 2003, supra; Schwartz et al., 1997, supra; Schwartz et al., 2003, supra).

### Example 6

### Transformation of Maize to Decrease Germination Stimulant Formation

### 6.1 Constructs

RNAi constructs were made as depicted in FIG. 9. Hereto, PCR fragments of 331 nucleotides were generated using PCR. Fragments were cloned into pUBlcas (modified pUC18) as an inverted repeat separated by an intron (FIG. 9). As promoters we used root-specific promoters. As an example we describe the construct for NCED. Primer 1 forward (intron 5′-onwards oligo introducing BamH I site): 5′GGATCCCTCCTGGGTCTCTGAGAT3′; primer 2 reverse (intron 5′-onwards oligo introducing Kpn I site): 5′GGTACCCCAGCCACACCCTCCTTT3′; primer 3 forward (sense fragment 5′-onwards oligo introducing BamH I site): 5′GGATCCCCACGATGATCCACGACTTC3′; primer 4 reverse (sense fragment 5′-onwards oligo introducing Bgl II site): 5′AGATCTATCTCGGTCAGCACGCTCTC3′; primer 5 forward (antisense fragment 5′-onwards oligo introducing Sal I site): 5′GTCGACCCACGATGATCCACGACTTC3′; primer 6 reverse primer 1 (antisense fragment 5′-onwards oligo introducing Kpn I site): 5′GGTACCATCTCGGTCAGCACGCTCTC3′ The constructs were made by cloning the promoters in pUC18 (the nos terminator already inside). Three promoters were used for the constructs, ubiquitin from maize and two root specific promoters pHm62 from rice and MtPT1 from red clover. Then the intron was generated from OsSHN1 genomic DNA using primer 1 and primer 2 and inserted into the vector. Then the 331 bp sense fragment of NCED was inserted (obtained using primer 3 and 4). Finally the 331 bp antisense fragment (obtained using primer 5 and 6) was ligated into the vector.

### 6.2 Plant Transformation

Maize inbred lines A188 und H99 are transformed essentially as described by Brettschneider et al. (1997; Theor Appl Genet. 94, 737-748). Particle bombardment of the scutellar tissue of immature embryos is carried out using a plasmid containing the pat (phosphinothricin acetyltransferase) gene as the selection marker and the plasmid with the construct. The co-transformation frequency is between 70-80% depending on the size of the construct. Bombardment is carried out with a PDS 1000/He gun (BioRad) using optimal parameters, such as the amount of gold particles used per bombardment, particle velocity, preculture time of the scutellum prior to bombardment and osmotic treatment of the target tissue before and after bombardment (Brettschneider et al., 1997, supra). Transgenic regenerants are selected on medium containing Basta [glufosinate]. Transgenic plant lines are verified with PCR and Southern blots, to check copy number.

Primary transformants are assayed for the induction of germination of Striga and Orobanche as described above (Example 1). The recombinant plants result in a significant reduction of germination of both Orobanche and Striga seeds, indicating that the plants have enhanced resistance to parasitic weeds.

### Example 7

### Agrobacterium rhizogenes Transformation of Tomato for Quick Screening of Strigolactone Pathway Genes

In order to have a quick screening method for strigolactone pathway candidate genes, stable transformants are not required. Therefore we have adapted an Agrobacterium rhizogenes transformation protocol, developed by Limpens et al., 2004 (J Exp Botany, 55: 983-992) for use as a fast screening method for strigolactone biosynthetic pathway gene candidates. In this method RNAi constructs of candidate genes can be quickly made and transformed to create transgenic tomato roots. The effect of the gene knock/out can be efficiently evaluated using an in situ germination bioassay with Orobanche ramosa. The A. rhizogenes transformation, as additional advantage, usually results in transgenic and non-transgenic roots on the same seedling (that can be discriminated using a red fluorescent marker (see below), such that germination with wildtype root exudate and transgenic root exudate can be compared on the same seedling/plant.

For this method, tomato seeds were sterilised for 20 min in 2% hypochlorite, containing 0.02% (v/v) Tween 20 and washed 3×20 min in sterile demineralised water. Seeds were incubated in Petri-dishes on sterile 3 MM Whatmann paper wetted with Farhaeus medium without agar (Limpens et al. 2004, supra). Seeds were germinating and subsequently grown in a climate room at 21° C., 16/8 h light/darkness with the Petri-dish in vertical position. The roots of 11 days old seedlings were cut from the hypocotyl and the wound surface inoculated with A. rhizogenes containing the appropriate binary vector (see below). To increase infection efficiency, the hypocotyls were also wounded by needles immersed in a culture of the same A. rhizogenes. The seedlings were co-cultivated with the A. rhizogenes for 7 days at 21° C., 16/8 h light/darkness. Subsequently, the seedlings were transferred into Emergence medium, containing 300 μg ml⁻¹ Cefotaxime (Duchefa), without agar (Limpens et al., 2004, supra). The plates were partly covered by aluminium foil and seedlings grown at 21° C., 16/8 h light/darkness in vertical position for 7-14 days. In this time new roots were formed, partly co-transformed with T-DNA of the binary vector, partly not (control roots). The co-transformed roots can be discriminated by the presence of DsRED1.

Single plants with transformed as well as non-transformed adventitious roots were transferred into a square Petri dish placed under an angle of about 45° with a small hole at the top, through which the shoot protuded out of the Petri dish while the roots grow inside on 3 MM Whatmann paper wetted with Farhaeus medium. The Petri dish was sealed with parafilm and covered with aluminium foil. For germination bioassays a 40×15 mm sheet of sterile plastic foil covered with a 35×12 mm sheet of GFFP containing preconditioned O. ramosa seeds was placed on the 3 MM Whatmann paper but under the tomato roots (transformed and non-transformed). In this way any contact between the O. ramosa seeds and possibly accumulated germination stimulants in the Whatmann paper is avoided. The GFFP containing O. ramosa seeds was wetted with Farhaeus medium. Two to three days later the GFFP with Orobanche seeds was transferred into a new Petri dish to allow Orobache seeds to germinate. The germination of Orobanche seeds induced by transformed/non-transformed roots was scored. As an example of how candidate genes can be screened using this method, below we describe the cloning of RNAi constructs for LeNCED1 and a generic LeNCED RNAi construct (aimed to target all tomato NCEDs).

Genomic DNA was isolated from tomato plants, cultivar Moneymaker of 2 weeks old plants. The DNA was isolated from 2 youngest leaves using Tri Reagent (Sigma), according to the manufacturer's protocol. The LeNCED1gene was amplified from genomic DNA using nested PCR. The primer designed was based on the LeNCED1 gene sequences from the NCBI database.

| | |
|---|---|
| First PCR | |
| Forward 1 | CCATAATTCCACACTCTCCC |
| Reverse 1 | GGACGTATATTCTAAAACCATCCC |
| Second PCR | |
| Forward 2 | CTCTCCTCATTTCCCACCTC |
| Reverse 2 | GTCACAATCATGCCTGATTTGCC |

LeNCED1 was ligated into pRSET-A (Invitrogen) and transformed to E. coli using restriction sites introduced by PCR using primers:

| | |
|---|---|
| Forward | GTGACGGATCCATGGCAACTACTACTTCACA |
| Reverse | GTGACGAATTCTCATGCCTGATTTGCCAAAT |

The tomato NCED fragments used for RNAi silencing were amplified by PCR using the isolated and purified LeNCED1-gene as a template. As a selection marker for co-transformed roots the gene coding for the fluorescent protein DsRED1 is used as a non-destructive selectable marker

Primer design based on conserved region (NCEDs)

| | |
|---|---|
| Forward | CACCGTCGGCTAGTTACGCTTG |
| Reversed | CTTGAGGTATGGCTTCTG |

Primer design based on non-conserved region
(LeNCED1)

| | |
|---|---|
| Forward | CACCTGGCTATCGCTGAACCAT |
| Reversed | CACAGTTGCCTCCAACTT |

Amplified sequences from LeNCED1 for RNAi constructs:

For conserved region
CACCGTCGGCTAGTTACGCTTGCCGTTTCACTGAAACAGAGAGGCTTGTT
CAAGAAAAAGCTTTGGGTCGCCCTGTTTTCCCTAAAGCCATTGGTGAATT
ACATGGTCACTCTGGAATTGCAAGGCTTATGCTGTTTTACGCTCGTGGGC
TCTTCGGACTTGTTGATCACAGTAAAGGAACTGGTGTTGCAAACGCCGGT
TTAGTCTATTTCAATAACCGATTACTTGCTATGTCTGAAGATGATTTGCC
TTACCATGTAAAGGTAACACCCACCGGCGATCTTAAAACAGAGGGTCGAT
TCGATTTCGACGGCCAGCTAAAATCCACCATGATAGCTCACCCAAAGCTC
GACCCAGTTTCCGGTGAGCTATTTGCTCTTAGCTACGATGTGATTCAGAA
GCCATACCTCAAG
For specific region
CACCTGGCTATCGCTGAACCATGGCCAAAAGTTTCTGGTTTTGCAAAAGT
AAACCTGTTCACCGGTGAAGTTGAGAAATTCATTTATGGTGACAACAAAT
ATGGTGGGGAACCTCTTTTTTTACCAAGAGACCCCAACAGCAAGGAAGAA
GACGATGGTTATATTTTAGCTTTCGTTCACGATGAGAAAGAATGGAAATC
AGAACTGCAAATTGTTAACGCAATGAGTTTGAAGTTGGAGGCAACTGTG

The fragments were cloned into pENTR-D-TOPO (Invitrogen) and recombined into modified Gateway pK7GWIWG2(II)-Q10:DsRED binary vector (Limpens et al., 2005, PNAS, 102: 10375-10380), which was then used to transform A. rhizogenes and subsequently tomato (see above).

### Example 8

### Transformation of Rice to Select the Carotenoid Cleavage Enzyme Involved in Strigolactone Formation

The carotenoid cleavage enzyme family in rice (Oryza sativa) was classified into 7 clusters containing 15 putative genes (SEQ ID 21, 23, 25, 27, 29, 31, 33, 35, 37, 39, 41, 43, 45, 47 and 49). In order to test which of these genes is involved in the formation of the strigolactones, we designed RNAi constructs for all of these genes. The inverted repeat regions (300 to 500 bps) for the constructs were amplified using specific primers and then subcloned into the pENTR/D-TOPO cloning vector (Invitrogen, Carlsbad, Calif.) to yield the entry vectors. The RNAi silencing constructs were made using the pANDA vector (Maki and Shimamoto, 2004, Plant Cell Physiol. 45, 490-495, Maki and Shimamoto, 2005, Plant Physiol. 138, 1903-1913). The final RNAi vectors were developed by an LR clonase reaction between an entry vector and the pANDA vector. Transgenic rice plants were generated from transformed calli (cv Nipponbare) by selecting for hygromycin resistance. The protocol used was according to Ouwerkerk, Institute of Biology, Leiden University. The transgenic plants are being characterized using the Striga germination bioassays described in Examples 1 and 3 and show significantly reduced germination for one of the RNAi constructs.

1. A method for making a recombinant plant being resistant to one or more species of parasitic plants belonging to the genera Orobanche or Striga, said method comprising:
a. generating a gene silencing vector comprising a promoter active in plant cells operably linked to a sense and/or antisense nucleic acid sequence of a carotenoid or apocarotenoid pathway gene, said pathway gene encoding a phytoene synthase enzyme or an enzyme downstream of phytoene synthase,
b. transforming a plant cell, plant tissue or plant with the vector of step (a),
c. regenerating a recombinant plant from a transformed plant cell plant tissue or plant,
d. testing the germination of Orobanche and/or Striga seeds in the presence of tissue, tissue exudates or tissue extracts of the recombinant plants and
e. selecting a plant which results in a lower seed germination compared to a non-recombinant control plant,
wherein said recombinant plant produces reduced amounts of at least one strigolactone in the root tissue.

2. The method according to claim 1, wherein said carotenoid or apocarotenoid pathway gene is selected from the group consisting of: phytoene synthase, phytoene desaturase, carotene desaturase, carotene isomerase, lycopene cyclase, β-carotene hydroxylase, zeaxanthin epoxidase, neoxanthine synthase or any enzyme involved in carotenoid catabolism to strigolactone germination stimulants, such as carotenoid cleavage diooxygenase, 9-cis-epoxycarotenoid dioxygenase, cytochrom P450 hydroxylase, epoxidase, dehydrogenase, demethylase and D-ring coupling enzyme.

3. The method according to claim 1, wherein said carotenoid or apocarotenoid pathway gene is selected from any one of SEQ ID NO: 1, 3, 5, 7, 9, 11, 13, 15, 17, 19, 21, 23, 25, 27, 29, 31, 33, 35, 37, 39, 41, 43, 45, 47 and 49 or from a nucleic acid sequence having at least 70% sequence identity over the entire length to any one of these sequences.

4. The method according to claim 1, wherein the plant is selected from maize, rice, milet, sorghum, cowpea, tomato, tobacco, melon, rapeseed, pea and sunflower.

5-6. (canceled)

7. A recombinant plant comprising, integrated in its genome, a sense and/or antisense nucleic acid sequence of an enzyme involved in carotenoid biosynthesis or carotenoid catabolism to strigolactones operably linked to a root-specific promoter active in plant cells, characterized in that said plant produces reduced or enhanced amounts of strigolactones in the roots compared to control plants and wherein root exudates from said plant are capable of reducing or increasing, respectively the percentage of germination of Orobanche and/or Striga seeds by at least 20% compared to non-recombinant controls.

8. (canceled)

9. The recombinant plant according to claim 7, wherein said nucleic acid sequence is selected from a sequence encoding an enzyme selected from the group consisting of: phytoene synthase, phytoene desaturase, carotene desaturase, carotene isomerase, lycopene cyclase, beta-carotene hydroxylase, zeaxanthin epoxidase, neoxanthine synthase or any enzyme involved in carotenoid catabolism to strigolactones, such as carotenoid cleavage diooxygenase, 9-cis-epoxycarotenoid dioxygenase, cytochrom P450 hydroxylase, epoxidase, dehydrogenase, demethylase and D-ring coupling enzyme.

10. A method for identifying an enzyme involved in carotenoid catabolism to strigolactones, comprising:
a. contacting plants with one or more species of mycorrhizae,
b. identifying one or more genes that are downregulated in said plants by mycorrhizal colonization,
c. obtaining the cDNA of said downregulated genes, and optionally
d. using said cDNA to generate RNAi constructs, and optionally
e. transforming a plant or a plant root with said constructs and testing the percentage of germination of Orobanche and/or Striga seeds in the presence of said root exudate.

11. A method for identifying an enzyme involved in carotenoid catabolism to strigolactones, comprising:
a. growing plants under phosphate limitation,
b. identifying one or more genes that are upregulated in said plants by phosphate limitation,
c. obtaining the cDNA of said upregulated genes, and optionally
d. using said cDNA to generate a RNAi constructs, and optionally
e. transforming a plant or a plant root with said constructs and testing the percentage of germination of Orobanche and/or Striga seeds in the presence of said root exudate.

12-13. (canceled)

14. A method of reducing parasitic weed infestation, comprising: irrigating or spraying crop plants, or soil on which crop plants are to be sown, with a composition comprising at least one carotenoid or apocarotenoid biosynthesis inhibitor.

15. The use according to claim 14, wherein the carotenoid or apocarotenoid biosynthesis inhibitor is: fluridone, norflurazone, isoxaflutole, flurtamone, clomazone, fluorochloridone, pyridazinone, nicotinanilide, amitrole, naproxen or abamine, or a mixture of any of these.

16. (canceled)

17. A method for identifying a mycorrhiza—parasitic weed host plant combination said method comprising:
a) inoculating a plurality of parasitic weed host species and/or varieties with one or more mycorrhiza species, and
b) testing the germination of Orobanche and/or Striga seeds in the presence of root exudates or root extracts obtained from the mycorrhiza-colonized plants, and
c) identifying the mycorrhiza-parasitic weed host plant combination which results in significantly lower Orobanche and/or Striga seed germination compared to a control plant lacking said mycorrhiza.

18. The method according to claim 17, wherein the root exudates or root extracts from the identified mycorrhiza-parasitic weed host plant combination results in at least 5% less Orobanche and/or Striga seed germination than the control plant lacking said mycorrhiza.

19. The method according to claim 1, wherein the promoter is root-specific.

20. The method according to claim 19, wherein the tissue is root tissue.

21. The recombinant plant according to claim 8, wherein said nucleic acid sequence is selected from a sequence encoding an enzyme selected from the group consisting of: phytoene synthase, phytoene desaturase, carotene desaturase, carotene isomerase, lycopene cyclase, beta-carotene hydroxylase, zeaxanthin epoxidase, neoxanthine synthase or any enzyme involved in carotenoid catabolism to strigolactones, such as carotenoid cleavage diooxygenase, 9-cis-epoxycarotenoid dioxygenase, cytochrom P450 hydroxylase, epoxidase, dehydrogenase, demethylase and D-ring coupling enzyme.


SEQUENCE LISTING (50 sequences)

SEQ ID NO: 1 - DNA, 1233 bp, Zea mays
atggccatcatactcgtacgagcagcgtcgccggggctctccgccgccgacagcatcagccaccaggggactctccagtgctccaccctgctcaagacgaagaggccggcggcgcgccggtggatgccctgctcgctccttggcctccacccgtgggaggctggccgtccctcccccgccgtctactccagcctcgccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggaactatgttgatgacagaggagcggcgccgcgccatatgggccatctatgtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcagccattcagggacatgattgaagggatgaggagtgatcttaggaagacaaggtataacaacttcgacgagctctacatgtactgctactatgttgctggaactgtcgggttaatgagcgtacctgtgatgggcatcgcaaccgagtctaaagcaacaactgaaagcgtatacagtgctgccttggctctgggaattgcgaaccaactcacgaacatactccgggatgttggagaggatgctagaagaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaactgagctctcacaggctagcagatggccagtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctag

SEQ ID NO: 2 - protein, 410 aa, Zea mays
MAIILVRAASPGLSAADSISHQGTLQCSTLLKTKRPAARRWMPCSLLGLHPWEAGRPSPAVYSSLAVNPAGEAVVSSEQKVYDVVLKQAALLKRQLRTPVLDARPQDMDMPRNGLKEAYDRCGEICEEYAKTFYLGTMLMTEERRRAIWAIYVWCRRTDELVDGPNANYITPTALDRWEKRLEDLFTGRPYDMLDAALSDTISRFPIDIQPFRDMIEGMRSDLRKTRYNNFDELYMYCYYVAGTVGLMSVPVMGIATESKATTESVYSAALALGIANQLTNILRDVGEDARRGRIYLPQDELAQAGLSDEDIFKGVVTNRWRNFMKRQIKRARMFFEEAERGVTELSQASRWPVWASLLLYRQILDEIEANDYNNFTKRAYVGKGKKLLALPVAYGKSLLLPCSLRNGQT

SEQ ID NO: 3 - DNA, 2264 bp, Zea mays
ctccaaatgcggaggtctcgactcttctctcttcctccatctttatcatcgccccacgtacacacccaattcctcgcaactgggctcccccgcctccacgacactgccccccgtctcaagtccgccgcctccattcttcagctctcctatcctccgcctagaatatcttcatcggtattttaccaacctggatcaatttactcacgatactctgaagcgtatacatatgccatatgggaaatgacttcatagctgtgggttgtcttatggctccttgaatttgcagtagtctgcctgtacctattggctgaagcagagctgacccccactttatcaagagttgctcaacgatggacactggctgcctgtcatctatgaatattactggagctagccagacaagatcttttgcggggcaacttcctcctcagagatgttttgcgagtagtcactatacaagctttgccgtgaaaaaacttgtctcaaggaataaaggaaggagatcacaccgtagacatcctgccttgcaggttgtctgcaaggattttccaagacctccactagaaagcacaataaactatttggaagctggacagctctcttcattttttagaaacagcgaacgccccagtaagccgttgcaggtcgtggttgctggtgcaggattggctggtctatcaacagcgaagtatctggcagatgctggccataaacccatattgcttgaggcaagagatgttttgggtggaaaggtagctgcttggaaggatgaagatggagattggtacgagactgggcttcatatattttttggagcttatcccaacatacagaatctgtttggcgagcttaggattgaggatcgtttgcagtggaaagaacactctatgatattcgccatgccaaacaagccaggagaattcagccggttcgatttcccagaaactttgccagcacctataaatgggatatgggccatattgagaaacaatgaaatgcttacttggccggagaaggtgaagtttgcaatcggacttctgccagcaatggttggtggtcaaccttatgttgaagctcaagatggcttaaccgtttcagaatggatgaaaaagcagggtgttcctgatcgggtgaacgatgaggtttttattgcaatgtccaaggcactcaatttcataaatcctgatgagctatctatgcagtgcattttgattgctttgaaccgatttcttcaggagaagcatggttctaaaatggcattcttggatggtaatccgcctgaaaggctatgcatgcctattgttgatcacattcggtctaggggtggagaggtccgcctgaattctcgtattaaaaagatagagctgaatcctgatggaactgtaaaacacttcgcacttagtgatggaactcaaataactggagatgcttatgtttgtgcaacaccagtcgatatcttcaagcttcttgtacctcaagagtggagtgaaattacttatttcaagaaactggagaagttggtgggagttcctgttatcaatgttcatatatggtttgacagaaaactgaacaacacatatgaccaccttcttttcagcaggagttcacttttaagtgtctatgcagacatgtcagtaacctgcaaggaatactatgacccaaaccgttcaatgctggagttggtctttgctcctgcagacgaatggattggtcgaagtgacactgaaatcatcgatgcaactatggaagagctagccaagttatttcctgatgaaattgctgctgatcagagtaaagcaaagattcttaagtatcatattgtgaagacaccgagatcggtttacaaaactgtcccaaactgtgagccttgccggcctctccaaaggtcacctatcgaaggtttctatctagctggtgattacacaaagcagaaatacctggcttctatggaaggtgcagtcctatccgggaagctttgtgcccagtccatagtgcaggattatagcaggctcgcactcaggagccagaaaagcctacaatcaggagaagttcccgtcccatcttagttgtagttggctttagctatcgtcatccccactgggtgctatcttatctcctatttcaatgggaacccacccaatggtcatgttggagacaacacctgttatggtcctttgaccatctcgtggtgactgtagttgatgtcatattcggatatatatgtaaaaggacctgcatagcaattgttagaccttggaaaaaaa

SEQ ID NO: 4 - protein, 571 aa, Zea mays
MDTGCLSSMNITGASQTRSFAGQLPPQRCFASSHYTSFAVKKLVSRNKGRRSHRRHPALQVVCKDFPRPPLESTINYLEAGQLSSFFRNSERPSKPLQVVVAGAGLAGLSTAKYLADAGHKPILLEARDVLGGKVAAWKDEDGDWYETGLHIFFGAYPNIQNLFGELRIEDRLQWKEHSMIFAMPNKPGEFSRFDFPETLPAPINGIWAILRNNEMLTWPEKVKFAIGLLPAMVGGQPYVEAQDGLTVSEWMKKQGVPDRVNDEVFIAMSKALNFINPDELSMQCILIALNRFLQEKHGSKMAFLDGNPPERLCMPIVDHIRSRGGEVRLNSRIKKIELNPDGTVKHFALSDGTQITGDAYVCATPVDIFKLLVPQEWSEITYFKKLEKLVGVPVINVHIWFDRKLNNTYDHLLFSRSSLLSVYADMSVTCKEYYDPNRSMLELVFAPADEWIGRSDTEIIDATMEELAKLFPDEIAADQSKAKILKYHIVKTPRSVYKTVPNCEPCRPLQRSPIEGFYLAGDYTKQKYLASMEGAVLSGKLCAQSIVQDYSRLALRSQKSLQSGEVPVPS

SEQ ID NO: 5 - DNA, 2265 bp, Zea mays
ccctgccacgacgcccgcgacaaatccctgcgcgacggcatcttcgcctcccatcccctcccagcttcccctcccactccggccctcacacaaattgcccctcttcttctcctcctctttacacgctgccgaccacggctgccgccaaccacccgccccacccgtccaccgctgccgagtgctagccatttggagctgccgcgccatggcgtccgtggccgccaccaccacgctggcaccggcactcgccccgcgccgggcgcggccagggactgggctcgtgccgccgcgccgggcctcggccgtcgctgctcgctcgaccgtaacgtctccgacatggcgtcaacgctcccaaaggttattcccacccgagccagagcactacaggggcccgaagctcaaggtggccatcataggggcaggccttgcgggcatgtccaccgctgttgagctcttggaccagggccatgaggttgatttgtacgagtcccgtccgtttatcggtggcaaggttggctcctttgttgacaggcaaggaaaccatatcgagatggggctgcatgtgttcttcgggtgctacagcaatctcttccgcctcatgaagaaggttggcgctgataataatctgctggtgaaggaacatacccatacttttgtaaataaagggggcacgattggtgaacttgattttcggttcccggtgggagctccgttacatggcattcaagcattcctaagaactaatcagctcaaggtttatgataaagcaagaaatgcagttgctcttgcccttagtccagttgttcgggctctggttgatcctgatggtgcattgcagcaagtgcgggacttggatgatataagtttcagtgattggttcatgtccaaagggggtactcgggagagtatcacaagaatgtgggatcctgttcgttacgctttgggtttcattgactgtgataatatcagtgcacgttgcatgcttactattttcaccttgtttgccacaaagacagaggcatccctgttacgcatgttaaagggttcacctgatgtttacttaagtggtccaataaagaagtatataacagacaggggtggtaggtttcacttaaggtggggatgcagagaggttctctatgagaagtcacctgatggagagacctatgttaagggccttctactcaccaaggctacaagtagagagataatcaaagctgatgcatacgtcgcagcctgtgatgttccaggtatcaaaagattacttccatcagaatggagggagtgggaaatgtttgacaatatctacaagttagatggtgtccctgttgtcactgtccagctccgctacaacggatgggtcactgaacttcaagatttggagaaatcaagacaactgcaaagggcggttgggttggataaccttttgtacacggcggatgcagacttttcctgtttttcggaccttgctctctcatctcctgctgattactacattgaagggcaaggttccctgatccaagctgtgctgactcctggagatccatacatgccattgccaaacgaggagatcattagtaaggttcaaaagcaggttgtagaactgttcccatcttcccggggcttagaagttacatggtccagtgtggtaaagatcggacaatcgctgtaccgtgaggctcctggaaacgacccattcaggcctgatcagaagacgcccgttaaaaacttcttcctctctggatcttacacgaaacaggactacatcgacagcatggaaggagcaactctctccggcaggcgaacgtcggcctacatctgcggtgccggggaggagctgctggccctccgaaagaagctactcatcgacgacggcgagaaggcgctggggaacgttcaagtcctgcaggctagctgaacaacccctcctgcactgcagagaagcttggatctttccaaccacacatacatgctggaatggacaaaccaaccaaccattgtcttttctcgcttcagggtgctggcgattcccgcagcaacctcctgtgtatcgtatccaatttgagcattagatctgccccccccccctgcaggcgtttctttcctatccctgatccgagaagcagggtgtagtctaggtggctggcatacgggattacatcaggcagtgtgtaagttcagctggaactcgattggtaattgggatggatgattgatgatatatatatagcacacactgttcttgcgtcttgcaaaaaaaaaaaa

SEQ ID NO: 6 - protein, 570 aa, Zea mays
MASVAATTTLAPALAPRRARPGTGLVPPRRASAVAARSTVTSPTWRQRSQRLFPPEPEHYRGPKLKVAIIGAGLAGMSTAVELLDQGHEVDLYESRPFIGGKVGSFVDRQGNHIEMGLHVFFGCYSNLFRLMKKVGADNNLLVKEHTHTFVNKGGTIGELDFRFPVGAPLHGIQAFLRTNQLKVYDKARNAVALALSPVVRALVDPDGALQQVRDLDDISFSDWFMSKGGTRESITRMWDPVRYALGFIDCDNISARCMLTIFTLFATKTEASLLRMLKGSPDVYLSGPIKKYITDRGGRFHLRWGCREVLYEKSPDGETYVKGLLLTKATSREIIKADAYVAACDVPGIKRLLPSEWREWEMFDNIYKLDGVPVVTVQLRYNGWVTELQDLEKSRQLQRAVGLDNLLYTADADFSCFSDLALSSPADYYIEGQGSLIQAVLTPGDPYMPLPNEEIISKVQKQVVELFPSSRGLEVTWSSVVKIGQSLYREAPGNDPFRPDQKTPVKNFFLSGSYTKQDYIDSMEGATLSGRRTSAYICGAGEELLALRKKLLIDDGEKALGNVQVLQAS

SEQ ID NO: 7 - DNA, 1848 bp, Lycopersicon esculentum
atgtgtaccttgagttttatgtatcctaattcacttcttgatggtacctgcaagactgtagctttgggtgatagcaaaccaagatacaataaacagagaagttcttgttttgaccctttgataattggaaattgtactgatcagcagcagctttgtggcttgagttggggggtggacaaggctaagggaagaagagggggtactgtttccaatttgaaagcagttgtagatgtagacaaaagagtggagagctatggcagtagtgatgtagaaggaaatgagagtggcagctatgatgccattgttataggttcaggaataggtggattggtggcagcgacgcagctggcggttaagggagctaaggttttagttctggagaagtatgttattcctggtggaagctctggcttttacgagagggatggttataagtttgatgttggttcatcagtgatgtttggattcagtgataagggaaacctcaatttaattactcaagcattggcagcagtaggacgtaaattagaagttatacctgacccaacaactgtacatttccacctgccaaatgacctttctgttcgtatacaccgagagtatgatgacttcattgaagagcttgtgagtaaatttccacatgaaaaggaagggattatcaaattttacagtgaatgctggaagatctttaattctctgaattcattggaactgaagtctttggaggaacccatctacctttttggccagttctttaagaagccccttgaatgcttgactcttgcctactatttgccccagaatgctggtagcatcgctcggaagtatataagagatcctgggttgctgtcttttatagatgcagagtgctttatcgtgagtacagttaatgcattacaaacaccaatgatcaatgcaagcatggttctatgtgacagacattttggcggaatcaactaccccgttggtggagttggcgagatcgccaaatccttagcaaaaggcttggatgatcacggaagtcagatactttatagggcaaatgttacaagtatcattttggacaatggcaaagctgtgggagtgaagctttctgacgggaggaagttttatgctaaaaccatagtatcgaatgctaccagatgggatacttttggaaagcttttaaaagctgagaatctgccaaaagaagaagaaaatttccagaaagcttatgtaaaagcaccttcttttctttctattcatatgggagttaaagcagatgtactcccaccagacacagattgtcaccattttgtcctcgaggatgattggacaaatttggagaaaccatatggaagtatattcttgagtattccaacagttcttgattcctcattggccccagaaggacaccatattcttcacatttttacaacatcgagcattgaagattgggagggactctctccgaaagactatgaagcgaagaaagaggttgttgctgaaaggattataagcagacttgaaaaaacactcttcccagggcttaagtcatctattctctttaaggaggtgggaactccaaagacccacagacgataccttgctcgtgatagtggtacctatggaccaatgccacgcggaacacctaagggactcctgggaatgcctttcaataccactgctatagatggtctatattgtgttggcgatagttgcttcccaggacaaggtgttatagctgtagccttttcaggagtaatgtgcgctcatcgtgttgcagctgacttagggtttgaaaaaaaatcagatgtgctggacagtgctcttcttagactacttggttggttaaggacactagcatga

SEQ ID NO: 8 - protein, 615 aa, Lycopersicon esculentum
MCTLSFMYPNSLLDGTCKTVALGDSKPRYNKQRSSCFDPLIIGNCTDQQQLCGLSWGVDKAKGRRGGTVSNLKAVVDVDKRVESYGSSDVEGNESGSYDAIVIGSGIGGLVAATQLAVKGAKVLVLEKYVIPGGSSGFYERDGYKFDVGSSVMFGFSDKGNLNLITQALAAVGRKLEVIPDPTTVHFHLPNDLSVRIHREYDDFIEELVSKFPHEKEGIIKFYSECWKIFNSLNSLELKSLEEPIYLFGQFFKKPLECLTLAYYLPQNAGSIARKYIRDPGLLSFIDAECFIVSTVNALQTPMINASMVLCDRHFGGINYPVGGVGEIAKSLAKGLDDHGSQILYRANVTSIILDNGKAVGVKLSDGRKFYAKTIVSNATRWDTFGKLLKAENLPKEEENFQKAYVKAPSFLSIHMGVKADVLPPDTDCHHFVLEDDWTNLEKPYGSIFLSIPTVLDSSLAPEGHHILHIFTTSSIEDWEGLSPKDYEAKKEVVAERIISRLEKTLFPGLKSSILFKEVGTPKTHRRYLARDSGTYGPMPRGTPKGLLGMPFNTTAIDGLYCVGDSCFPGQGVIAVAFSGVMCAHRVAADLGFEKKSDVLDSALLRLLGWLRTLA

SEQ ID NO: 9 - DNA, 2276 bp, Zea mays
taggccagtcgcccagacacagagaggaaacatcgtctggtcattgtaaggcctgaataaacatggggtcggggcatggcaattcccggtaaccaccagcgcaatttggacggtttgaaaggtgacaagtagtgggggtaagcttccgatgctgcgctcgtggtggttgtgaaggccggcgttgtctcttacctgcgaaggccggcgcaggatctatcggtagaatcggccagcagcgttttcgtggacttgtccttcgtctttaaggccaaaagtaatattcaaaaaacatttgaggtgcttgggtgaagaatgccacaatggaattggatcgacggtggtgcccccactcaaagcagctgcagcttatcaccaccgcctcaggctcaggattgcacggctccgccgcgctcgaattccccccaccttccaccaggccagcaccatggccactaccgccctcctcctccgcactcaccaccatccctgcaagccgccggcgccgcgcgcgtccgtactctgccgcgccacggcggggatggctgggccggcgtcggccgcggcgctgcggtccctggccccgcccacgcggccggagctgctgtcgctcgacctgccccgctacgacccagcgcccgcccgccccgtggacctcgccgtggtgggcggcgggcccgcgggcctcgctgtggcgcagcgcgtcgcggaggcgggcctgtcggtgtgcgccatcgacccgtcccccgcggtcgtgtggcccaacaactacggcgtgtgggtggacgagttcgaggccatgggcctctcccactgcctcgacaccgtctggccctccgcctccgtcttcatcgacgatggcggcgccaagtcgctcgaccgcccctacgcccgcgtcgcgcgccgcaagctcaagtccaccatgatggaccgctgcgtcgccaacggcgtcgtcttccaccaggccaaggtcgccaaggccgtccactacgacgcctcgtccctcctcatctgtgacgacggcgtcgccgtcccggcaagcgtcgtgctcgacgccacgggcttctcgcgctgcctcgtgcagtacgacaagccgtacaacccggggtaccaggtcgcctacggcatcctcgccgaggtcgacgcccacccgttcgacatcgacaagatgctcttcatggactggcgcgactcgcacctccccgaagggtcggagatcagggagcgcaaccgccgcatccccaccttcctctacgccatgcccttctcccccaccaggatcttcctcgaggagacgtccctggtcgcgcgcccggggctcgccatggacgacatccaggagcgcatggccgcgcgcctcaggcacctgggtatacgcgtccgaagcgtcgaggaggacgagcgctgcgtcatccccatgggagggccgctgcccgtcctgccgcagagggttgtcggcatcggcggcacggcagggatggtgcacccgtccacgggctacatggtggcgcgcacgcttgccaccgcgcctatcgtggcggacgccatcgtaaggttcctcgacaccggcaccggcaacggcatgggtggcctggcaggggacgcgctctccgccgaggtgtggaagcagctgtggccagccaacaggcggcggcagagggagttcttctgcttcggcatggacgtcctgctcaagctggacctcgagggaacgcggcggttcttcgacgccttcttcgacctggagccacactactggcacggtttcctgtcatccagactgttcctgccggagctcttgatgttcggcctcgcactgttcgggaacgcctccaactcgtcgaggctggagatcatggccaagggcaccgtgcctcttggcaagatgattggcaacttgatacaggacagagatgggtgaggagggtatgtatacctacatttttcacgtgaagatctgatctccattggatctctgattttggtatcgatgattttcactgtatttacgatttgcaaagatggattcacaaaacacagttagcaacagcagttcaggacctcctgtcagatataggaattgctgctgcaacgctacttcagtatggtgattacagaggtgtatagttgcacttgcacactgagggatgtcgtgagaatctacgtatcagatatcatggtcttcatataaaagatcaaatttccaacaaaatatgaaattgacagttgtgtattatgatagggtgttttcgatatttcaagaacatagaaagaacggg

SEQ ID NO: 10 - protein, 490 aa, Zea mays
MATTALLLRTHHHPCKPPAPRASVLCRATAGMAGPASAAALRSLAPPTRPELLSLDLPRYDPAPARPVDLAVVGGGPAGLAVAQRVAEAGLSVCAIDPSPAVVWPNNYGVWVDEFEAMGLSHCLDTVWPSASVFIDDGGAKSLDRPYARVARRKLKSTMMDRCVANGVVFHQAKVAKAVHYDASSLLICDDGVAVPASVVLDATGFSRCLVQYDKPYNPGYQVAYGILAEVDAHPFDIDKMLFMDWRDSHLPEGSEIRERNRRIPTFLYAMPFSPTRIFLEETSLVARPGLAMDDIQERMAARLRHLGIRVRSVEEDERCVIPMGGPLPVLPQRVVGIGGTAGMVHPSTGYMVARTLATAPIVADAIVRFLDTGTGNGMGGLAGDALSAEVWKQLWPANRRRQREFFCFGMDVLLKLDLEGTRRFFDAFFDLEPHYWHGFLSSRLFLPELLMFGLALFGNASNSSRLEIMAKGTVPLGKMIGNLIQDRDG

SEQ ID NO: 11 - DNA, 879 bp, Oryza sativa
atggccgtcgcgaggctggtggtcatcacccccgccgtcctcctcggccgcaccgcccgcgtctcgccgtcggcggtgccgcggctgcggcccatcgtcgccggccgccgcgccgtggcggcgcccacacgcgccgtcctgggagacggggcgggtgtcggcggcgaggaggatgcggtggtggcggtggtggaggaggacgcggtcgcccggcgcgcggcgaggaagcggtcggagcggcgcacgtacctggtggcggcggtgatgtccagcctcgggttcacgtccatggccgccgccgccgtctactaccgcttcgcctggcaaatggaggccggcggcggcgacgttccggcgacggagatggtcggcacgttcgcgttgtcggtgggggcggcggtggggatggagttctgggcgcggtgggcgcaccgggcgctgtggcacgcgtcgctgtggcacatgcacgagtcgcaccaccgcccgcgcgacggcccgttcgagctcaacgacgtcttcgccatcgccaacgccgccccggccatctccctcctcgcctacggcctcctcaaccgcggcctcctccccggcctctgcttcggcgcggggcttgggattacgctgttcgggatggcgtacatgttcgtccacgacggcctggtccaccggcgcttccccgtggggcccatcgagaacgtgccctacttccgccgagttgctgccgcccaccagatacatcacacggacaagttcgaaggcgtgccctacggcctgttcctcggacccaaggagttggaggaggtgggtgggactgaggagctggacaaggagatcaagaagaggatcaagaggaaggaggccatggacgccatcagatga

SEQ ID NO: 12 - protein, 292 aa, Oryza sativa
MAVARLVVITPAVLLGRTARVSPSAVPRLRPIVAGRRAVAAPTRAVLGDGAGVGGEEDAVVAVVEEDAVARRAARKRSERRTYLVAAVMSSLGFTSMAAAAVYYRFAWQMEAGGGDVPATEMVGTFALSVGAAVGMEFWARWAHRALWHASLWHMHESHHRPRDGPFELNDVFAIANAAPAISLLAYGLLNRGLLPGLCFGAGLGITLFGMAYMFVHDGLVHRRFPVGPIENVPYFRRVAAAHQIHHTDKFEGVPYGLFLGPKELEEVGGTEELDKEIKKRIKRKEAMDAIR

SEQ ID NO: 13 - DNA, 1881 bp, Oryza sativa
atggcgctcctctccgcgaccgcccccgccaagacgcgcttctcgctcttctcccacgaggaggcgcagcacccgcacccgcacgcgctctcggcgtgctgcggcggcggcgccagcggcaagaggcagcgggcgcgggccagggtggcggcggcaatgcggccggcggacgcggccgcctccgtcgcgcaggcggcgtcgccgggcggcggcggcgagggcacgcggaggccgcgggtgctcgtggccggcggcggcatcggggggctggtgctggcgctggcggcgaggcggaaggggtacgaggtgacggtgttcgagcgcgacatgagcgcggtgcgcggcgaggggcagtaccgcgggccgatacagatccagagcaacgcgctcgcggcgctggcggccatcgacatgtccgtcgccgaggaggtcatgcgcgaaggctgcgtcaccggcgaccgcatcaacggcctcgtcgacggcatctccggctcctggtacatcaagtttgatacatttactcctgcagctgagcgaggtctcccagttacaagggttattagccgaatgacgctgcagcagattcttgctcgtgcggttggtgatgatgctatactgaacgatagccatgttgttgatttcatagatgatggcaacaaggtaactgcaattttggaggatggccggaaatttgaaggtgaccttttggttggtgctgatggaatatggtcaaaggtgaggaaggtgcttttcgggcaatcagaagcaacttattcagaatatacttgctacactggcattgcagactttgtgcctcctgacattgacacagttgggtaccgtgtatttcttggtcacaaacaatatttcgtctcctcagatgtcggtgctggaaaaatgcagtggtatgcatttcataaggaacctgctggtggcactgatcctgaaaatggtaaaaataaaagattgctcgagatatttaatggttggtgcgataacgtcgttgatctgataaatgcaactgatgaggaagcaattcttcgccgggatatatatgaccgaccacctacttttaactggggaaaaggtcgtgtcactttgctaggtgactctgtacatgctatgcagccaaatctgggtcaaggtggctgcatggctattgaggatggttaccagctggctgtagaacttgagaagtcctggcaggagagtgcgaagtctggaactcctatggatatagtttcctccttgaggagatatgagaaggagagaatactacgtgtttctgttatacatgggttggcaagaatggcagcaatcatggctaccacttatagaccatacttgggtgtgggtctgggaccattgtcgtttttaacgaagttgaggataccacatcctggaagagttggtgggagattcttcatcaagtatggaatgcctttgatgttgagctgggttctaggaggaaacagcacgaagttagaaggaagaccgttaagctgtaggctttctgacaaggcaaacgaccagcttcgtcgatggtttgaggatgacgatgcattggaacaagccatgggtggagagtggtacctcctccccacaagttctggagactcgcaacccattcgattaatcagggatgaaaaaaagtcactctccattggaagccggtcagatcccagcaattcgactgcttccctggcattgcccttgccacagatatcagaaaaccatgctactatcacatgcaagaataaggccttttatgtgactgataatggaagtgaacatggtacatggattaccgacaacgaaggtagacgctatcggcgtacctccgaacttccctgtccgtttccatccctcggatgccattga

SEQ ID NO: 14 - protein, 626 aa, Oryza sativa
MALLSATAPAKTRFSLFSHEEAQHPHPHALSACCGGGASGKRQRARARVAAAMRPADAAASVAQAASPGGGGEGTRRPRVLVAGGGIGGLVLALAARRKGYEVTVFERDMSAVRGEGQYRGPIQIQSNALAALAAIDMSVAEEVMREGCVTGDRINGLVDGISGSWYIKFDTFTPAAERGLPVTRVISRMTLQQILARAVGDDAILNDSHVVDFIDDGNKVTAILEDGRKFEGDLLVGADGIWSKVRKVLFGQSEATYSEYTCYTGIADFVPPDIDTVGYRVFLGHKQYFVSSDVGAGKMQWYAFHKEPAGGTDPENGKNKRLLEIFNGWCDNVVDLINATDEEAILRRDIYDRPPTFNWGKGRVTLLGDSVHAMQPNLGQGGCMAIEDGYQLAVELEKSWQESAKSGTPMDIVSSLRRYEKERILRVSVIHGLARMAAIMATTYRPYLGVGLGPLSFLTKLRIPHPGRVGGRFFIKYGMPLMLSWVLGGNSTKLEGRPLSCRLSDKANDQLRRWFEDDDALEQAMGGEWYLLPTSSGDSQPIRLIRDEKKSLSIGSRSDPSNSTASLALPLPQISENHATITCKNKAFYVTDNGSEHGTWITDNEGRRYRRTSELPCPFPSLGCH

SEQ ID NO: 15 - DNA, 1497 bp, Solanum tuberosum
atggaaacccttctaaagcctttgacatctcttttactttcctctcctacaccacacaggtctattttccaacaaaatcccccttctctaaatcccaccaccaaaaaaaaatcaagaaaatgtcattttagaaacgaaagcagtaaacttttttgtagctttcttgatttagcacccatatcaaagccagagtcttttgatgttaacatctcattggttgatcctaattcaggtcgggctcaattcgacgtgatcatcatcggagctggccctgctggtctcaggctagctgaacacgtttctaaatatggtattaaggtatgttgtgttgacccttcaccactttccatgtggccaaataattatggtgtttgggttgatgagtttgagaatttaggattggaagattgtttagatcataaatggcctatgacttgtgtacatataaatgatcataaaactaagtatttgggaagaccatatggtagagtaagtagaaagaagctgaagttgagattgttgaatagttgtgttgaaaacagagtgaagttttataaagctaaggtttggaaagtggaacatgaagaatttgagtcttcaattgtttgtgatgatggtaagaagataagaggtagtttggttgttgatgcaagtggttttgctagtgattttatagagtatgacaagccaagaaaccatggttatcaaattgctcatggggttttagtagaagttgataatcatccatttgatttggataaaatggtgcttatggattggagggattctcatttaggtaatgagccatatttaagggtgaataatgctaaagaaccaacattcttgtatgcaatgccatttgatagaaatttggttttcttggaggagacttctttggtgagtcgtcctgtgttatcgtatatggaagtaaaaagaaggatggtagcaagattaaggcatttggggatcaaagtgagaagtgttattgaggaagagaaatgtgtgatccctatgggaggaccacttccgcggattcctcaaaatgttatggctattggtgggaattcggggatagttcatccatcgacagggtacatggtggctaggagcatggctttggcaccagtactggctgaagccatcgtcaaggggcttggctcaacaagaatgataagagggtctcaactttaccatagagtttggaatggtttgtggcctttggatagaagatgtattggagaatgttattcatttgggatggagacattgttgaagcttgatttgaaagggactaggagattgtttgatgctttctttgatcttgatcctaaatactggcaagggttcctttcttcaagattgtctgtcaaagaacttgctatactcagcttgtgtctttttggacatggctcaaatttgactaggttggatattgttacaaaatgtcctgttcctttggttagactgattggcaatctagcaatagagagcctttga

SEQ ID NO: 16 - protein, 498 aa, Solanum tuberosum
METLLKPLTSLLLSSPTPHRSIFQQNPPSLNPTTKKKSRKCHFRNESSKLFCSFLDLAPISKPESFDVNISLVDPNSGRAQFDVIIIGAGPAGLRLAEHVSKYGIKVCCVDPSPLSMWPNNYGVWVDEFENLGLEDCLDHKWPMTCVHINDHKTKYLGRPYGRVSRKKLKLRLLNSCVENRVKFYKAKVWKVEHEEFESSIVCDDGKKIRGSLVVDASGFASDFIEYDKPRNHGYQIAHGVLVEVDNHPFDLDKMVLMDWRDSHLGNEPYLRVNNAKEPTFLYAMPFDRNLVFLEETSLVSRPVLSYMEVKRRMVARLRHLGIKVRSVIEEEKCVIPMGGPLPRIPQNVMAIGGNSGIVHPSTGYMVARSMALAPVLAEAIVKGLGSTRMIRGSQLYHRVWNGLWPLDRRCIGECYSFGMETLLKLDLKGTRRLFDAFFDLDPKYWQGFLSSRLSVKELAILSLCLFGHGSNLTRLDIVTKCPVPLVRLIGNLAIESL

SEQ ID NO: 17 - DNA, 2498 bp, Zea mays
caacaacagactacggaggaccgccgccgcccacgcaaaaacccacccgatccccgcaaaaacccccgcacgcgatccacgccaattccagtcaccgcgatgcagggtctcgccccgcccacctctgtttccatacaccggcacctgccggcccggtccagggcccgggcctccaattccgtcaggttctcgccgcgcgccgtcagctccgtgccgcccgccgagtgcctccaggcgccgttccacaagcccgtcgccgacctgcctgcgccgtccaggaagcccgccgccattgccgtcccagggcacgccgcggcgccgaggaaagcggagggcggcaagaagcagctcaacttgttccagcgcgccgcggcggccgcgctcgacgcgttcgaggaagggttcgtggccaacgtcctcgagcggccccacgggctgcccagcacggccgacccggccgtgcagatcgccggcaacttcgcgcccgtcggggagaggccgcccgtgcacgagctccccgtctccggccgcatcccgcccttcatcgacggggtctacgcgcgcaacggcgccaacccctgcttcgaccccgtcgcggggcaccacctcttcgacggcgacggcatggtgcacgcgctgcggatacgcaacggcgccgccgagtcctacgcctgccgcttcacggagaccgcgcgcctgcgccaggagcgcgcgatcggccgccccgtcttccccaaggccattggcgagctgcacgggcactccgggatcgcgcgcctcgccctgttctacgcgcgcgccgcgtgcggcctcgtggacccctcggccggcaccggcgtggccaacgccggcctcgtctacttcaacggccgcctgctcgccatgtccgaggacgacctcccctaccacgtccgcgtggcggacgacggcgacctcgagaccgtcggccgctacgacttcgacgggcagctcggctgcgccatgatcgcgcaccccaagctggacccggccaccggggagctccacgcgctcagctacgacgtcatcaagaggccgtacctcaagtacttctacttcaggcccgacggcaccaagtccgacgacgtggagatcccgctggagcagcccacgatgatccacgacttcgccatcaccgagaacttcgtggttgtgcccgaccaccaggtggtgttcaagctccaggagatgctgcgcggcgggtcgcccgtggtgctggacaaggagaagacgtcgcggttcggcgtgctccccaagcacgccgcggacgcgtcggagatggcgtgggtggacgtgccggactgcttctgcttccacctgtggaacgcgtgggaggacgaggcgacgggcgaggtggtggtgatcggctcctgcatgacccccgccgactccatcttcaacgagtccgacgagcgcctggagagcgtgctgaccgagatccgcctggacgcgcgcacgggccggtccacgcgccgcgccgtcctgccgccgtcgcagcaggagaacctggaggtgggcatggtgaaccgcaacctgctgggccgcgagagccggtacgcgtacctcgcggtggcggagccgtggcccaaggagtcgggcttcgccaaggaggacctgtccacgggcgagctcaccaagttcgagtacggcgagggccggttcggcggcgagccctgcttcgttcccatggacccggccgcggcccacccgcgcggcgaggacgacgggtacgtgctcaccttcgtccacgacgagcgcgccggcacgtcggagctacttgtggtcaatgccgccgacatccggctggaggccacggttcagctgccgtcccgcgtgcccttcggcttccacggcaccttcatcacgggccaggagctcgaggcccaggcggcctgaccgagctccacgtttctcggaggaggaacagaggagccagccttggatcaggggagaagtcaccagagggagcccagatcagttccccggggtcttcctgtccccccacgcacaccagagttacagttggtagttgctttgctttgcctttttttaacattatttcatttgacactagtgtaagtaaattggtatagtggcagcttagagagagagagagattagtagaaaggcgcccagctcgtagcttaacagctggtggtgcctctggctagtatagagtacatatttttttttcttcttctgtgctttatcccttttttttttctccctttggatgacatggatgtgcatccagctccagttgttgtcacccagtccagtggactggggtgtctgggatcttggttgctgttacttgctcgccattgccacccagttgctgccgctgctgctgaggtgttgctggcttggcttgtatactctgtgttccctgtggctatcggtgcgtgtacatttgttcacagtatatgactgatggtattaaaataagaaccggtgacggcttctgtttcaaaaaaaaa

SEQ ID NO: 18 - protein, 604 aa, Zea mays
MQGLAPPTSVSIHRHLPARSRARASNSVRFSPRAVSSVPPAECLQAPFHKPVADLPAPSRKPAAIAVPGHAAAPRKAEGGKKQLNLFQRAAAAALDAFEEGFVANVLERPHGLPSTADPAVQIAGNFAPVGERPPVHELPVSGRIPPFIDGVYARNGANPCFDPVAGHHLFDGDGMVHALRIRNGAAESYACRFTETARLRQERAIGRPVFPKAIGELHGHSGIARLALFYARAACGLVDPSAGTGVANAGLVYFNGRLLAMSEDDLPYHVRVADDGDLETVGRYDFDGQLGCAMIAHPKLDPATGELHALSYDVIKRPYLKYFYFRPDGTKSDDVEIPLEQPTMIHDFAITENFVVVPDHQVVFKLQEMLRGGSPVVLDKEKTSRFGVLPKHAADASEMAWVDVPDCFCFHLWNAWEDEATGEVVVIGSCMTPADSIFNESDERLESVLTEIRLDARTGRSTRRAVLPPSQQENLEVGMVNRNLLGRESRYAYLAVAEPWPKESGFAKEDLSTGELTKFEYGEGRFGGEPCFVPMDPAAAHPRGEDDGYVLTFVHDERAGTSELLVVNAADIRLEATVQLPSRVPFGFHGTFITGQELEAQAA

SEQ ID NO: 19 - DNA, 1928 bp, Zea mays
ggccactccccttccatctcgtcccttcgctacaagtcatctcgccgcaaccggaacccgcagccatggggacggaggcggagcagccggacatggacagccaccgaaacgacggcgtcgtggtggtgccagcgccgcgcccgcgtaaggggctcgcctcctgggcgcttgacctgctcgagtccctcgccgtgcgcctcggccacgacaagaccaagccgctccactggctctccggcaacttcgcccccgtcgtcgaggagaccccgccggccccaaaccttagcgtccgcggacacctcccggagtgcttgaatggagagtttgtcagggttgggcctaatccgaagtttgctcctgttgcggggtatcactggtttgatggagacgggatgattcatgccatgcgtattaaggatggaaaagctacctatgtatcaagatatgtgaagactgcccgcctcaaacaagaggagtattttggtggagcaaagtttatgaagattggagaccttaagggattttttggattgtttatggtccaaatgcagcaacttcggaaaaaattcaaagtcttggattttacctatggatttgggacagctaatactgcacttatatatcatcatggtaaactcatggccttgtcagaagcagataagccatatgttgttaaggtccttgaagatggagacttgcagactcttggcttgttggattatgacaaaaggttgaaacattcttttactgcccatccaaaggttgacccttttacagatgaaatgttcacattcggatattcacatgaacctccatactgtacataccgtgtgattaacaaagaaggagctatgcttgatcctgtgccaataacaataccggaatctgtaatgatgcatgattttgccatcacagagaattactctatttttatggacctccctttattgttccgaccaaaggaaatggtgaagaacggtgagtttatctacaagtttgatcctacaaagaaaggtcgttttggtattctcccccgctatgcaaaggatgacaaactcatcagatggtttcaactccctaattgtttcatattccataatgctaatgcttgggaagagggtgatgaagttgttctcattacctgccgccttgagaatccagatttggacaaggtgaatggatatcaaagtgacaagctcgaaaacttcgggaatgagctgtacgagatgagattcaacatgaaaacgggtgctgcttcacaaaagcaactgtctgtttctgctgtggattttcctcgtgttaatgagagctatactggcagaaagcagcggtatgtctactgcactatacttgacagcattgcgaaggtgactggcatcataaagtttgatctgcatgctgaaccggaaagtggtgtgaaagaacttgaagtgggaggaaatgtacaaggcatatatgacctgggacctggtagatttggttcagaggcaatttttgttcccaagcatccaggtgtgtctggagaagaagatgacggctatttgatattctttgtacacgacgagaacacagggaaatctgaagtaaatgttatcgatgcaaagacaatgtctgctgatccagttgcggtggttgagcttcctaatagggttccttatggattccatgccttttttgtaactgaggaccaactggctcgacaggcggaggggcagtgaagatacggcacctgcagattctgcacacgcgggtacaggttggaaattattgcagaggacatgtatatgtataggacaagtttattacacatgtattcgaaccacacattacacaagtttattgcaggacgtgtattcgaaccacacattacacatgtatatgtataggacaagtttattacagtcctgattgattgcaatttgcaaatt

SEQ ID NO: 20 - protein, 550 aa, Zea mays
MGTEAEQPDMDSHRNDGVVVVPAPRPRKGLASWALDLLESLAVRLGHDKTKPLHWLSGNFAPVVEETPPAPNLSVRGHLPECLNGEFVRVGPNPKFAPVAGYHWFDGDGMIHAMRIKDGKATYVSRYVKTARLKQEEYFGGAKFMKIGDLKGFFGLFMVQMQQLRKKFKVLDFTYGFGTANTALIYHHGKLMALSEADKPYVVKVLEDGDLQTLGLLDYDKRLKHSFTAHPKVDPFTDEMFTFGYSHEPPYCTYRVINKEGAMLDPVPITIPESVMMHDFAITENYSIFMDLPLLFRPKEMVKNGEFIYKFDPTKKGRFGILPRYAKDDKLIRWFQLPNCFIFHNANAWEEGDEVVLITCRLENPDLDKVNGYQSDKLENFGNELYEMRFNMKTGAASQKQLSVSAVDFPRVNESYTGRKQRYVYCTILDSIAKVTGIIKFDLHAEPESGVKELEVGGNVQGIYDLGPGRFGSEAIFVPKHPGVSGEEDDGYLIFFVHDENTGKSEVNVIDAKTMSADPVAVVELPNRVPYGFHAFFVTEDQLARQAEGQ

SEQ ID NO: 21 - DNA, 1827 bp, Oryza sativa
atggcgacgatcacgacgccaggatatgctcacatacagcggcagcacggcaggtgctcgacgacggcgggaaggcgtggggcgtccaattcggtgagattctccgcgcgcgcggttagctccgtgccgcacgcggcggcggcgtcatcggcgccggcgttcctgccggtgccgttcgtgcctggggccgacgcaccgtcgccgtcggggaagagtgccattggcgtcccgaaggcgccgaggaagggggaggaggggaagaggctcaacttcttccagcgcgccgcggcgatggcgctcgacgcgttcgaggaggggtttgtggcgaatgtcctcgagcgcccgcacgggctgccgagcacggccgaccccgcggtgcagatcgccggcaacttcgcgccggtcggtgagacgccgcccgcgcgcgcgctgccggtgtcggggcgcatcccgcccttcatcaacggcgtctacgcgcgcaacggcgccaacccgcacttcgaccccgtcgccgggcaccacctgttcgacggcgatggcatggtgcacgccgtcaggatacgcaacggcgccgccgagtcgtacgcgtgccggttcacggagaccgcgcggctgcggcaggagcgcgcgatggggcggcccatgttccccaaggccattggggagctccatggccactccggcatcgcgcgccttgctctgttctacgcgcgcgccgcctgcggcctcctcgacccgtcacacggcaccggcgtcgccaacgccggcctcatctacttcaacggcaggctcctcgccatgtcggaggacgacctcccctaccaggtgcgcgtcaccgccgacggcgacctcgagaccgtcggccgctacgacttcgacgggcagctcggctgcgccatgatcgcgcaccccaagctcgacccggccaccggagagctccacgcgctcagctacgacgtgatcaagaagccgtacctcaagtacttctacttcgcgcccgacggcaccaagtcggccgacgtcgagatcccgctcgaccagcccaccatgatccacgacttcgccatcaccgagaactacgtggtggtacccgaccaccaggtggtgttcaagctccaggagatgctccgcggcggctcgcccgtggtgctcgacaaggagaagacgtcgcggttcggggtgctccccaagcacgccgcggacgcgtcggagatggtgtgggtggacgtcccggactgcttctgcttccacctctggaacgcgtgggaggaggcggacaccgacgaggtggtggtgatcggctcgtgcatgacccccgccgactccatcttcaacgagtccgacgaccgcctcgagagcgtcctcaccgagatccgcctcaacacccgcaccggcgagtcgacgcggcgcgccatcctgccgccgtcgagccaggtcaacctcgaggtgggcatggtcaaccgcaacctcctcggccgcaagacgcggtacgcctacctcgccgtggccgagccgtggcccaaggtgtcgggcttcgccaaggtggacctcgccacgggtgagctcaccaagttcgagtacggcgagggccggttcggcggcgagccctgcttcgtccccatggacgccgccgccgccacgccccgcggcgaggacgacggctacatcctgtccttcgtccacgacgagcgcgccgggacctccgagctcctcgtcgtcaatgccgccgacatgcgccttgaggccaccgtgcagctgccgtcccgcgtgccgtacggcttccacggcacgttcatcaccggcgacgagctcaccacccaggcctga

SEQ ID NO: 22 - protein, 608 aa, Oryza sativa
MATITTPGYAHIQRQHGRCSTTAGRRGASNSVRFSARAVSSVPHAAAASSAPAFLPVPFVPGADAPSPSGKSAIGVPKAPRKGEEGKRLNFFQRAAAMALDAFEEGFVANVLERPHGLPSTADPAVQIAGNFAPVGETPPARALPVSGRIPPFINGVYARNGANPHFDPVAGHHLFDGDGMVHAVRIRNGAAESYACRFTETARLRQERAMGRPMFPKAIGELHGHSGIARLALFYARAACGLLDPSHGTGVANAGLIYFNGRLLAMSEDDLPYQVRVTADGDLETVGRYDFDGQLGCAMIAHPKLDPATGELHALSYDVIKKPYLKYFYFAPDGTKSADVEIPLDQPTMIHDFAITENYVVVPDHQVVFKLQEMLRGGSPVVLDKEKTSRFGVLPKHAADASEMVWVDVPDCFCFHLWNAWEEADTDEVVVIGSCMTPADSIFNESDDRLESVLTEIRLNTRTGESTRRAILPPSSQVNLEVGMVNRNLLGRKTRYAYLAVAEPWPKVSGFAKVDLATGELTKFEYGEGRFGGEPCFVPMDAAAATPRGEDDGYILSFVHDERAGTSELLVVNAADMRLEATVQLPSRVPYGFHGTFITGDELTTQA

SEQ ID NO: 23 - DNA, 1842 bp, Oryza sativa
atgccgaccaccttcacgcccaattcccccgcctcctcgtgttccatacaccaccgcgcctccccgtcgaggggtgcccgcaattcggtgcggttcacgcgcccgcgcgccgccgccgcggcgacgaactcggtgctcagcgcgccgtcgtccgtgccgcccgcgtacgtgccgccgccgccgccgccgccgaccaagatgttcccggaggcgggcgacgcggcggcggccaaggctgcggcgaggaggtgtggcaagaagaaggatgggctgaacttcttccagcgcgcggcggcggtggcgctcgacgcgttcgaggaagggttcatcacgaatgtgctggagaggccgcacgcgctgccgcggacggccgacccggcggtgcagatcgccgggaacttcgcgccggtgggggagcagccgccggtgcggtcgctcccggtgtccggccgcatcccgcccttcatcaatggcgtctacgcccgcaacggcgccaacccgcacttcgagcccaccgcgggccaccacctgttcgacggcgacggcatggtccacgccgtccgcatccgcaacggcgccgccgagtcctacgcctgccgcttcaccgagactgcgcgcctcggccaggagcgcgccctcggccgcgccgtcttccccaaggccatcggcgagctccatggccactccggcatcgcccgcctcgccctcttctacgcgcgggggctctgcggcctcgtcgacccgtcgcacggcaccggcgtcgccaacgccggcctcgtctacttcaacggccgcctcctcgccatgtccgaggacgacctcccgtaccaggtccgcgtcaccgccgacggcgacctcgagacggtggggcgctacgacttcgacggccagctcggctgcgccatgatcgcccaccccaagctcgacccggtctccggcgagctcttcgccctcagctacgacgtgatcaagaagccatacctgaaatacttctacttcgacgccgatggcaccaagtcacccgacgtcgagatcgagcttgagcagccgacgatgatccacgacttcgccatcaccgagaacttcgtggtggtacccgaccaccaggtggtgttcaagctcggcgagatgttccgcggcggctcgccggtggtgctcgacagggagaagacgtcgcggttcggcgtgctccccaagcacgcgacgagctcgttggagatggtgtgggtcgacgtccccgactgcttctgcttccacctgtggaatgcgtgggaggaggccgagtccggcgaggtggtggtggtgggatcctgcatgacgcccgccgactccatcttcaacgagtcggacgaacacctcgagagcgtgctcaccgagatccgcctcaacacgcgcaccggcgagtccacccgccgcgccgtgctgccgccggcggcgcaggtgaacctcgaggtcggcatggtgaaccgcgccatgctcggccggaagacgaggtacgcctacctcgccgtcgccgagccgtggcccaaggtgtccggcttcgccaaggtggacctcgccaccggcgagctcaccaagttcgagtacggcgagggccggttcggcggcgagccgtgcttcgtgcccatgggcggcgccggcgccgccgcgtccccggcgcgcggcgaggacgacggctacatcctctccttcgtccgcgacgaggccgcgggcacatccgagctcctcgtcgtgaacgccgccgacatgaggctggaggccaccgtccagctgccgtcgcgcgtcccctacggcttccacggcaccttcatcaacgccggcgagctcgccacgcaggcctag

SEQ ID NO: 24 - protein, 613 aa, Oryza sativa
MPTTFTPNSPASSCSIHHRASPSRGARNSVRFTRPRAAAAATNSVLSAPSSVPPAYVPPPPPPPTKMFPEAGDAAAAKAAARRCGKKKDGLNFFQRAAAVALDAFEEGFITNVLERPHALPRTADPAVQIAGNFAPVGEQPPVRSLPVSGRIPPFINGVYARNGANPHFEPTAGHHLFDGDGMVHAVRIRNGAAESYACRFTETARLGQERALGRAVFPKAIGELHGHSGIARLALFYARGLCGLVDPSHGTGVANAGLVYFNGRLLAMSEDDLPYQVRVTADGDLETVGRYDFDGQLGCAMIAHPKLDPVSGELFALSYDVIKKPYLKYFYFDADGTKSPDVEIELEQPTMIHDFAITENFVVVPDHQVVFKLGEMFRGGSPVVLDREKTSRFGVLPKHATSSLEMVWVDVPDCFCFHLWNAWEEAESGEVVVVGSCMTPADSIFNESDEHLESVLTEIRLNTRTGESTRRAVLPPAAQVNLEVGMVNRAMLGRKTRYAYLAVAEPWPKVSGFAKVDLATGELTKFEYGEGRFGGEPCFVPMGGAGAAASPARGEDDGYILSFVRDEAAGTSELLVVNAADMRLEATVQLPSRVPYGFHGTFINAGELATQA

SEQ ID NO: 25 - DNA, 1749 bp, Oryza sativa
atggcgtcctccgcgccttccgcccccggcctcgcgccggtcgccaagccgccgccgccgccgtccaaggtgaaggtggcgacagcaaccgtgccaaccaatggcaagatcaagcagggtgcgaggccaatgcgtgtctcggcgccgccggtggagccgcggcggcggatgaacccgctccagcggctggcggcggcggcgattgacgccgtggaggaaggcctcgtcgccgggttgctcgagcgggggcacgcgctgccgcgcaccgctgatccggccgtgcagatcgccgggaactacgcgcccgtcggggagcgcccgccggtgagggggctgccggtgtccggccgcctcccggcgtgcctcgacggggtgtacgtccgcaacggcgccaacccgctccacgcgccgcgcgccgggcaccacctgttcgacggcgacgggatgctgcacgccgtgcggctcgccggggggcgcgccgagtcgtacgcgtgccggttcacggagacggcgcggctgcggcaggagcgggagatgggacgccccgtgttccccaaggccatcggcgagctccacggccactccggcgtcgcgcggcttctgctgttcggctcgcgcgcgctctgcggcgtgctcgacgcgtcccggggcatcggcgtcgccaacgccggcctcgtctaccacgacggccgcctcctcgccatgtccgaggacgacctcccctaccacgtccgtgtcacccacgacggcgacctcgagaccgtcgggaggtacgacttccatgggcagctcgacgccgacggcaccatgatcgcgcaccccaagctcgacccggtcaccggcgagctcttcgcgctcagctacaatgtcgtgtccaagccgtacctcaagtacttctacttcaccgccgacggccgcaagtcccgagacgtcgacatccccgtcggcgcgccgacgatgatccacgacttcgccgtcaccgagaactatgccgtcgtccccgaccagcagatcgtgttcaagctccaggagatggtgcgcggcggctcgccggtggtatacgacagggagaaggcgtcgcggttcggcgtgctcccgaagcgcgccgccgacgcgtcggagctccggtgggtggaggtccccggctgcttctgcttccacctctggaacgcgtgggaggacgacgccaccggcgagatcgtggtcatcggctcctgcatgacgccgccggacgccgtgttcaacgagccgtcgcagtcgccggaggaggagagcttccgcagcgtgctctccgagatccgcctcgacccgcgcaccggcgtgtcgcggcggcgcgacgtgctgcgcgacgccgccgagcaggtgaacctcgaggccggcatggtgaaccggcagctgctcggccggaagacgcggtacgcctacctcgccatcgccgagccatggccgagggtgtcgggcttcgccaaggtggacctcgagagcggcacggcggagaagttcatctacggcgaggggaggtacggcggcgagccatgcttcgttccgcgcgccggcgccgcggcggaggacgacggccacgtgctgtgcttcgtccacgacgaggagcgcggcacgtcggagctggtggtggtggacgccggcagcgaggcgatggaggaggtcgcggccgtgaagctgccggggcgcgtgccgtacggattgcacggcaccttcattggcgccaacgagctgcagcgacaagcttag

SEQ ID NO: 26 - protein, 582 aa, Oryza sativa
MASSAPSAPGLAPVAKPPPPPSKVKVATATVPTNGKIKQGARPMRVSAPPVEPRRRMNPLQRLAAAAIDAVEEGLVAGLLERGHALPRTADPAVQIAGNYAPVGERPPVRGLPVSGRLPACLDGVYVRNGANPLHAPRAGHHLFDGDGMLHAVRLAGGRAESYACRFTETARLRQEREMGRPVFPKAIGELHGHSGVARLLLFGSRALCGVLDASRGIGVANAGLVYHDGRLLAMSEDDLPYHVRVTHDGDLETVGRYDFHGQLDADGTMIAHPKLDPVTGELFALSYNVVSKPYLKYFYFTADGRKSRDVDIPVGAPTMIHDFAVTENYAVVPDQQIVFKLQEMVRGGSPVVYDREKASRFGVLPKRAADASELRWVEVPGCFCFHLWNAWEDDATGEIVVIGSCMTPPDAVFNEPSQSPEEESFRSVLSEIRLDPRTGVSRRRDVLRDAAEQVNLEAGMVNRQLLGRKTRYAYLAIAEPWPRVSGFAKVDLESGTAEKFIYGEGRYGGEPCFVPRAGAAAEDDGHVLCFVHDEERGTSELVVVDAGSEAMEEVAAVKLPGRVPYGLHGTFIGANELQRQA

SEQ ID NO: 27 - DNA, 1917 bp, Oryza sativa
atgcaaaggatttgccctgctcactgctcggtcactcactcactcaccatgaagtccatgaggctttcctacatccctcctgctgcttctgctgctccacagagccccagctatggcaggaagaagaacgcctccgccgctccgccatcggctgccgcctccaccaccgttctcacctccccgctggtgaccaccacccgcactccgaagcagaccgagcaagaggacgagcagttggtagccaagaccaagactacgagaactgttattgctacgacgaatggcagggcggcgccgagccagtctcggcctcgccgccggcctgcccccgccgccgcggcgtcggccgcttcgctgccgatgacgttctgcaacgcgctggaggaggtgatcaacacgttcatcgacccgccggcgcttcggccggcggtggacccgcggaacgtgctgaccagcaacttcgtgcccgtggacgagctgccgccgacgccctgccccgtcgtgcgcggcgccatcccgcgctgcctcgccggcggcgcctacatccgcaacgggcccaacccgcagcacctcccgcgcgggccgcaccacctgttcgacggcgacggcatgctgcactccctcctcctcccgtcgcccgcgtcgtccggcgacgaccccgtcctgtgctcgcgctacgtgcagacgtacaagtacctcgtggagcgcgacgccggcgcgcccgtcctgcccaacgtcttctccggcttccacggcgtggccgggatggcgcgcggcgccgtcgtggcggccagggtcctgaccgggcagatgaatccgttggagggcgtcgggctcgccaacaccagcctcgcctacttcgccggccgcctctacgcgctcggcgagtccgacctcccctacgccgtgcgcgtccacccggacaccggcgaggtgaccacgcacggcaggtgcgacttcggcggccgcctcgtcatgggcatgaccgcgcaccccaagaaggaccccgtcaccggcgagctcttcgcgttccgctacggccccgtgccgccgttcgtgacgtacttccggttcgacccggccggcaacaagggcgccgacgtgcccatcttctccgtgcagcagccgtcgttcctgcacgacttcgccatcaccgagcggtacgccatcttcccggagatccagatcgtgatgaagcccatggacatggtggtgggcggcggctcgcccgtggggtcggaccccggcaaggtgccccgcctcggcgtgatcccgcgctacgccaccgacgagtcggagatgcggtggttcgaggtgccgggcttcaacatcatgcactcggtgaacgcgtgggaggaggccggcggcgaggagctggtgctggtggcgcccaacgtcctctccatcgagcacgccctggagcacatggagctagtgcactcctgcgtcgagaaggtgcgcatcaacctccgcaccggcgtcgtcacgcgcaccccgctcgccgccgggaacttcgacttccccgtgatcaacccggctttcctcggccgccgcaacaggtacggctacttcggcgtcggcgaccccgcgcccaagatcggcggcgtggccaagctcgacttcgaccgcgccggcgagggcgactgcaccgtggcgcagcgcgacttcgggcccgggtgcttcgccggcgaaccgttcttcgtggccgacgacgtcgagggcaacggcaacgaggatgacgggtacttggtgtgctacgtccacgacgaggccaccggcgagaaccggttcgtggtgatggacgcgcggtcgccggacctggagatcgtcgcggaggtgcagctgcccggacgcgtcccctacggcttccatggcctgttcgtcacgcaggccgagctccagtcacagcaccaatga

SEQ ID NO: 28 - protein, 638 aa, Oryza sativa
MQRICPAHCSVTHSLTMKSMRLSYIPPAASAAPQSPSYGRKKNASAAPPSAAASTTVLTSPLVTTTRTPKQTEQEDEQLVAKTKTTRTVIATTNGRAAPSQSRPRRRPAPAAAASAASLPMTFCNALEEVINTFIDPPALRPAVDPRNVLTSNFVPVDELPPTPCPVVRGAIPRCLAGGAYIRNGPNPQHLPRGPHHLFDGDGMLHSLLLPSPASSGDDPVLCSRYVQTYKYLVERDAGAPVLPNVFSGFHGVAGMARGAVVAARVLTGQMNPLEGVGLANTSLAYFAGRLYALGESDLPYAVRVHPDTGEVTTHGRCDFGGRLVMGMTAHPKKDPVTGELFAFRYGPVPPFVTYFRFDPAGNKGADVPIFSVQQPSFLHDFAITERYAIFPEIQIVMKPMDMVVGGGSPVGSDPGKVPRLGVIPRYATDESEMRWFEVPGFNIMHSVNAWEEAGGEELVLVAPNVLSIEHALEHMELVHSCVEKVRINLRTGVVTRTPLAAGNFDFPVINPAFLGRRNRYGYFGVGDPAPKIGGVAKLDFDRAGEGDCTVAQRDFGPGCFAGEPFFVADDVEGNGNEDDGYLVCYVHDEATGENRFVVMDARSPDLEIVAEVQLPGRVPYGFHGLFVTQAELQSQHQ

SEQ ID NO: 29 - DNA, 1731 bp, Oryza sativa
atggaggtacccattgctgccatgacttttgcccacccagccaatgttatgactctggcttcaaggcagccaaagagtaaaaggtcccatatctcccctgctaccacggctcaccgtaatctacagactcgcctggctcaccaccaccatgcaacaccagcttcattgcctatggcaatctgcaacacagtagacaaagtgatcaataggttcattgacctgccggagcagcgaccaacggtggatccgcggcgtgtgctctctggcaacttcgctcctgttgatgagctgcccccgacaagctgccatgtcatccgcggctccatcccaagctgcctcgccggtggggtctacatccgcaatggtcccaacccacagcaccggcttccccagcgaacacaccacctcttcgatggtgatggcatgctccactcccttctcattccctcggcctcgtcaacactgttgtcggagcctgtgctttgttcacgctatgtgcacacgtacaagtatctcttggagcgtgagaccggaggaccggttttaccaaacttcttcgctggcttccatggagtggccggcttggctcgtgcagtggtcatgatcgcaagagtgcttgctggtcaaattaacctgaacaagggcttcgggctggccaacactagcatcactctttttgcagattgcctatatgcgctatgcgaatctgaccttccctactccatgcacatcaacccagccaacggagaagtcaccacacttggtcgatgtgactttggtggtgatctttcttttaggatgacagcacaccccaagaaggacccggtcaccatggagttgtttgcttttcgctacaatgtcttccaaccattcataacatacttctggttcgatcgagcaggcagcaaggtcgcagatgtgcccatcttgtccttgcagaaaccatcggtgatgcatgactttgcaataacagagagatatgcaatctttccagagtcacaactcatcgttaatcccatggacatggtcatgcgggggagctcgttggtaggattggaccgtaccatggtgccacggattggcgtgcttccaaggtacgccaaggatgagtcagacatgagatggtttgaggtgcctagatttaatatgttgcacacgacgaatggttgggaagaggctgatggagaggagattgtgctcgtggcacccaatatcctatctatcgaacacatgctaggaaacatggagctcatgcgagctcgtgtcgacatggtacgtatcaacctctgcaccggtgacgtgtcgtgcactgcactctcaccggagagccttgagttcggtgtcatccaccaaggttatgttggtcgcaaaaatcgctatggctactttggtgtaagtggtccgttgcccaagatcaaggggataagaaagcttgactttgatctcgtcggctctggtgattgcacggttggacgtcgtgactttggtctagggtgctttgctggggaaccattttttgttccagacaacatcgacgggtatggaaacgaggatagtggttatgtggtgtgctacacccatgaagaggacaccggagagagttggtttgtggtgatggatgcaaagtctccagagctagacattgttgcagaagtgcaacttcctagtcgtatcccctatggctttcatggtatttttgtcaaacaggccgaacttctcgcacaacaataa

SEQ ID NO: 30 - protein, 576 aa, Oryza sativa
MEVPIAAMTFAHPANVMTLASRQPKSKRSHISPATTAHRNLQTRLAHHHHATPASLPMAICNTVDKVINRFIDLPEQRPTVDPRRVLSGNFAPVDELPPTSCHVIRGSIPSCLAGGVYIRNGPNPQHRLPQRTHHLFDGDGMLHSLLIPSASSTLLSEPVLCSRYVHTYKYLLERETGGPVLPNFFAGFHGVAGLARAVVMIARVLAGQINLNKGFGLANTSITLFADCLYALCESDLPYSMHINPANGEVTTLGRCDFGGDLSFRMTAHPKKDPVTMELFAFRYNVFQPFITYFWFDRAGSKVADVPILSLQKPSVMHDFAITERYAIFPESQLIVNPMDMVMRGSSLVGLDRTMVPRIGVLPRYAKDESDMRWFEVPRFNMLHTTNGWEEADGEEIVLVAPNILSIEHMLGNMELMRARVDMVRINLCTGDVSCTALSPESLEFGVIHQGYVGRKNRYGYFGVSGPLPKIKGIRKLDFDLVGSGDCTVGRRDFGLGCFAGEPFFVPDNIDGYGNEDSGYVVCYTHEEDTGESWFVVMDAKSPELDIVAEVQLPSRIPYGFHGIFVKQAELLAQQ

SEQ ID NO: 31 - DNA, 522 bp, Oryza sativa
atgaggtggttcgacgtgccgggctgcttctgcttccacatctggaacgcctgggacgaacccgccgtcgtcatcgtctgctcctgtatcacgccgcccgacgcgctcttgtcctccgtgcgcgccgtcctgtccgaggtccggctcgacctccggacggggcggtacagccggcgcgagctcgtgccagggctcaacctcgaggccggcacggtgaaccggtcgctgctcggccgccgcactcgcttcacctacctcgccgtcgccgagccgtggccgcggtgccgcggcgtggccaaggtggaccttggcaccggcgagctcgcggccgtgcacgagtacggcgagggacggttcagcggcgagcccacgttcgtgccggcgacgtcggcgacgtccggcaccggcaccggaggcagggaggacgacgggcacgtggtggtgatggtccacgacgaggcggcgggcacggcggagctggtggtgctcgacgccgggaagatggaggtggcggcgacgtag

SEQ ID NO: 32 - protein, 173 aa, Oryza sativa
MRWFDVPGCFCFHIWNAWDEPAVVIVCSCITPPDALLSSVRAVLSEVRLDLRTGRYSRRELVPGLNLEAGTVNRSLLGRRTRFTYLAVAEPWPRCRGVAKVDLGTGELAAVHEYGEGRFSGEPTFVPATSATSGTGTGGREDDGHVVVMVHDEAAGTAELVVLDAGKMEVAAT

SEQ ID NO: 33 - DNA, 1992 bp, Oryza sativa
atggagagcacgtgctctccaatttatcctactaatataacacaagaaacgcaatcacgactgaaaaggagaccaaaaacggtgccatccatgtatacccttcaaccacgcgtatgcatctccagctccaggtcatccatctcccctaaagctgctcgtctatctcatcagaaagcatccactggaaagccgtatttcagggagattcaggtgcatctaagctccaaactgggagaggcatcaaatgccatgaacagtacttaccaacagctgctggattcatttgttgatcacaccttcacattcaaatgtcaacctttgcgtcccactgagagcaattttgcaccagtcgacgagatcggggagatcacacgggttatagagattgaaggagagattccagcagattttcctgagggtgtctacataagaaatggcggaaaccctctctatggaggcctccaatctgttagttccattttcgggcagtcccataacatctgggtggaaggagaaggcatgctccatgctgtatacttctgcaaaagtaataacagcacatggtcaatctcgtacaataaccgctacgtgcaatctgaaacatttaggattgagaaagaacgtcagaaaccatgctttctccctatgaccgatgggaaccctcccgccatgctcatcgcctctgttcttaacacagtatggtgcctgctgctattactacatcgcatttcttctgtaatttttttgggattgtcgcaaacattttggaaatgctatatgttgactgttgagtatgtgcagctgagattccgaaaggtaatgaaaagcatgagcaacactagcgtgtttgagcatgcagggagagtttatgcagcatcagaggatgatgttccccacgaggttgacttgcataacctcagtacactaggcagctggcatctcggtggtgaatggaagttgccatttacagcacaccccaaggtaatccctgggtcaaaagagatggtcattttcgggatcaatgcggtacagcctttcctaacagttggcataatctcagaggatggagaaaaacttaaacaaaaggttggtctcaagctagacagatgcacatattgtcatgaaattggggtcacggggacgtacaacatcatcatagattcaccacttaccctcaacccaactaggatgctaagaggggctccagtgcttgagtttgaagaggaaagctattcaagaattggggttatgccccattacggtgatgcagactcagtaatatggttttatgtagaaccattctgcacattccatcttgtcaattgcttcgaggaaggtcatgaggttgtagtaaggggatttcatgtaccaagttctgcgattatgggtccaaggcagaagaacatggttatggatacttccagtcaagaaccaaatgaagagaacttttctcgcttgtatgaatggagattgaacctaaaaacaaggactgttgcaggcaaatacttaactagcttagatgttgccttggaattcccagttatcaatgataaattttctggcctacgccataggtacgcatatgtacaagtggcggattgttcagcttgtttcggaggtggtcatgaaatagaaactaatttcagtctaaatcttgcagctcgaccaaagtttattggttttgcaaaactttgtcttgaagaaaaacaaaacatagctactaagatagacagagaagatctaataaaggtagagtatcatcagctagcaaaaaatcaattctgctcaggagtaacgtttgtgcctaaggcagcaggtgcgcatgaagatgatggctggatagtttcatttgttcatgatgaggaaaccaatatatcaaaagtacacatcattgacgcccgaaactttgagagtgaacctatagccaagataatattaccacaaagagtaccctatggtttgcatggagcattcattacaaaaagaacatga

SEQ ID NO: 34 - protein, 663 aa, Oryza sativa
MESTCSPIYPTNITQETQSRLKRRPKTVPSMYTLQPRVCISSSRSSISPKAARLSHQKASTGKPYFREIQVHLSSKLGEASNAMNSTYQQLLDSFVDHTFTFKCQPLRPTESNFAPVDEIGEITRVIEIEGEIPADFPEGVYIRNGGNPLYGGLQSVSSIFGQSHNIWVEGEGMLHAVYFCKSNNSTWSISYNNRYVQSETFRIEKERQKPCFLPMTDGNPPAMLIASVLNTVWCLLLLLHRISSVIFLGLSQTFWKCYMLTVEYVQLRFRKVMKSMSNTSVFEHAGRVYAASEDDVPHEVDLHNLSTLGSWHLGGEWKLPFTAHPKVIPGSKEMVIFGINAVQPFLTVGIISEDGEKLKQKVGLKLDRCTYCHEIGVTGTYNIIIDSPLTLNPTRMLRGAPVLEFEEESYSRIGVMPHYGDADSVIWFYVEPFCTFHLVNCFEEGHEVVVRGFHVPSSAIMGPRQKNMVMDTSSQEPNEENFSRLYEWRLNLKTRTVAGKYLTSLDVALEFPVINDKFSGLRHRYAYVQVADCSACFGGGHEIETNFSLNLAARPKFIGFAKLCLEEKQNIATKIDREDLIKVEYHQLAKNQFCSGVTFVPKAAGAHEDDGWIVSFVHDEETNISKVHIIDARNFESEPIAKIILPQRVPYGLHGAFITKRT

SEQ ID NO: 35 - DNA, 1659 bp, Oryza sativa
atggcgacctccctgaccctgatcgccacaccatgcacggctcctagatcatcctcgtccttcgcactcgctccacggctgccgcctcgttgcagcaacgcgactgcagcacgtcgccgcgccgtgagggcaaccacgctgcaatcggatcaggaacctgcaggatcaggagacagcggcgccacgacgacgaagctgtctgcctcgaccagcgtccgccaagaacgctgggaaggcgacctgcccatcgagggatgcctccctccttggctgaatggcacgtacatcaggaacgggccggggatgtgggacgtcggggagcacgcgttccaccacctgttcgacggctacgcgacgctcgtccgcgtctccttccgcggcgggggtggtgcgcgcgccacgggggcgcaccggcagatcgagtcggaggcgtacagggccgccgtggcgcgcggccgcccggtcctccgcgagttctcccactgccccgcgccggccaagagcctgctccaccgcttcggcgatctcgtcggcctcgtcaccggcgccgcgctcaccgacaaccccaacagcgccgtgctgccgctcggcgacggccgggtgatgtgcctcaccgagaccaccaagagctccgtcctcatcgacccggacacgctcgagacggtgggcaggttccggtacacggacaggctgggtggcatggtgcagtcggcgcacccgatcgtgaccgacacggagttcctgacgctgctgccggacctcgtccgtccgggccacctcgtcgtgaggatggaggccgggagcaacgagaggaaggtgatcgggagaatggactgccgcggcgggccgtcgcccgggtggctgcactcgttcgccgtcacggagaagtacgccgtcgtgccggagatgccgctccggtactcctccgccagcctgctcgcctccgagctcgccccgttctacgccttcgactgggtcccggcgtccggcagctacatgcacgtcatgtgcaagtccaccggcaagaccgtggcgagcgtggaggtgcctcctttcatggcgatccacttcatcaacgcatacgaggaggaaggcgacgaggccgccgtcgttgtcgactgctgtgagcactacggtgaccctgccatcatcgagacactcgtcctcagtagactgagattgttaaggggcaaggacgttttacctaacgccagggtggggcggttcaggatcccgctggatgggagcccgttcggcgaacttgagacggcgctggacccggaggagcacgggcgggggatggacatgtgcagcatcaacccggcgcgcctcggcaggaagtaccagtatgcctacgcgtgcggcgcgcgtcgcccgtgcaacttccccaacacgctcaccaagatcgacctggtggagaagaaggccaagagctggcacgaggagggctccgtgccgtccgagcctttcttcgtcgcgaggccgggagccaccgacgaagacgatggagtggtgatatctattgtaagctccgacgatggcgaagggtacgcactggtgctagacgcgaccacgtttgaggagattgcgcgcgtcaggttcccctacggattgccctacggcttccatggctgctggatccctgcgacggaagagtga

SEQ ID NO: 36 - protein, 552 aa, Oryza sativa
MATSLTLIATPCTAPRSSSSFALAPRLPPRCSNATAARRRAVRATTLQSDQEPAGSGDSGATTTKLSASTSVRQERWEGDLPIEGCLPPWLNGTYIRNGPGMWDVGEHAFHHLFDGYATLVRVSFRGGGGARATGAHRQIESEAYRAAVARGRPVLREFSHCPAPAKSLLHRFGDLVGLVTGAALTDNPNSAVLPLGDGRVMCLTETTKSSVLIDPDTLETVGRFRYTDRLGGMVQSAHPIVTDTEFLTLLPDLVRPGHLVVRMEAGSNERKVIGRMDCRGGPSPGWLHSFAVTEKYAVVPEMPLRYSSASLLASELAPFYAFDWVPASGSYMHVMCKSTGKTVASVEVPPFMAIHFINAYEEEGDEAAVVVDCCEHYGDPAIIETLVLSRLRLLRGKDVLPNARVGRFRIPLDGSPFGELETALDPEEHGRGMDMCSINPARLGRKYQYAYACGARRPCNFPNTLTKIDLVEKKAKSWHEEGSVPSEPFFVARPGATDEDDGVVISIVSSDDGEGYALVLDATTFEEIARVRFPYGLPYGFHGCWIPATEE

SEQ ID NO: 37 - DNA, 756 bp, Oryza sativa
atgctacatgactttgcaatcacggagcattatgccatcttccctgagtcgcaacttgtcatgtgtccgatgaacatggctctacgtggtggctcgctgataggattggatagtgcaatggtacaacggattggtgtgctaccaaggtatgccgaggacgagtcagagatgaggtggttcaagctaatgcctgggaggaagctaatggagaggagattatattggtgtcaaccaacaacttatctgtcacacatgcttggcaacatggaactcatgcaagttcaggttgacatgatacatatcaatctccgcactggtaccgttttgcgcactgcactctcactggagagccttgagtttggtgtaattcatcagggctacgtcggtcgatacaaccgctatggatactttggtgtaagcgcaccgttacccaggttctctggaatcagaaagctcgactttgccatggttggtgctgacgactgcacggttgcacgccgtgactttggccccgggtgctttgttggggagccatttttcgtgccaagcaatgataatggggatggtaatgaggacaatggatatgtggtgtgctatacacacaaagaggataccggagagagccagttcgtggtgatggatgccatgtctccagaactggaaattgttgcagcggtgcagctgcctgcccgtgttccctatggctttcatggtcgttttatcacgcaggccgaacttttgtcgcagcagaaataa

SEQ ID NO: 38 - protein, 251 aa, Oryza sativa
MLHDFAITEHYAIFPESQLVMCPMNMALRGGSLIGLDSAMVQRIGVLPRYAEDESEMRWFKLMPGRKLMERRLYWCQPTTYLSHMLGNMELMQVQVDMIHINLRTGTVLRTALSLESLEFGVIHQGYVGRYNRYGYFGVSAPLPRFSGIRKLDFAMVGADDCTVARRDFGPGCFVGEPFFVPSNDNGDGNEDNGYVVCYTHKEDTGESQFVVMDAMSPELEIVAAVQLPARVPYGFHGRFITQAELLSQQK

SEQ ID NO: 39 - DNA, 969 bp, Oryza sativa
atgcaaaggatttgccctgctcactgctcggtcactcactcactcaccatgaagtccatgaggctttcctacatccctcctgctgcttctgctgctccacagagccccagctatggcaggaagaagaacgcctccgccgctccgccatcggctgccgcctccaccaccgttctcacctccccgctggtgaccaccacccgcactccgaagcagaccgagcaagaggacgagcagttggtagccaagaccaagactacgagaactgttattgctacgacgaatggcagggcggcgccgagccagtctcggcctcgccgccggcctgcccccgccgccgcggcgtcggccgcttcgctgccgatgacgttctgcaacgcgctggaggaggtgatcaacacgttcatcgacccgccggcgcttcggccggcggtggacccgcggaacgtgctgaccagcaacttcgtgcccgtggacgagctgccgccgacgccctgccccgtcgtgcgcggcgccatcccgcgctgcctcgccggcggcgcctacatccgcaacgggcccaacccgcagcacctcccgcgcgggccgcaccacctgttcgacggcgacggcatgctgcactccctcctcctcccgtcgcccgcgtcgtccggcgacgaccccgtcctgtgctcgcgctacgtgcagacgtacaagtacctcgtggagcgcgacgccggcgcgcccgtcctgcccaacgtcttctccggcttccacggcgtggccgggatggcgcgcggcgccgtcgtggcggccagggtcctgaccgggcagatgaatccgttggagggcgtcgggctcgccaacaccagcctcgcctacttcgccggccgcctctacgcgctcggcgagtccgacctcccctacgccgtgcgcgtccacccggacaccggcgaggtgaccacgcacggcaggtgcgacttcggcggccgc

SEQ ID NO: 40 - protein, 323 aa, Oryza sativa
MQRICPAHCSVTHSLTMKSMRLSYIPPAASAAPQSPSYGRKKNASAAPPSAAASTTVLTSPLVTTTRTPKQTEQEDEQLVAKTKTTRTVIATTNGRAAPSQSRPRRRPAPAAAASAASLPMTFCNALEEVINTFIDPPALRPAVDPRNVLTSNFVPVDELPPTPCPVVRGAIPRCLAGGAYIRNGPNPQHLPRGPHHLFDGDGMLHSLLLPSPASSGDDPVLCSRYVQTYKYLVERDAGAPVLPNVFSGFHGVAGMARGAVVAARVLTGQMNPLEGVGLANTSLAYFAGRLYALGESDLPYAVRVHPDTGEVTTHGRCDFGGR

SEQ ID NO: 41 - DNA, 1437 bp, Oryza sativa
atggtggatggtgtggtgaccttactagaggcaaggaagcttgtggggaaggtgatcctagcggggagtgctaatttatatgatggatctatccatgagcagagcaatttcgcaccagttgatgaaatcggtggaagaacagagatatggaggatagaagggacgatctcagatgattttcctgagggtgtttatatcagaaatggttccaaccctctctttggagccttgcacaaggtgaactcaatcttcggccagtctgaagacatctgggtcgagggagaaggcatgctgcatgccctctacttcaccaagagcagagaaggaaacacttggtcagtctcatacaacaaccgctacgtgcaatctgacacttttaatactgaaagggatcgtcagagaccatgcttcctctcagcaatcaagggtgaccctcttgccataatcgcagccagcattttgaacatgctgaggtttggcaaggtgtttagaaacatgagtaataccggtgtgtttgagcacgccgagagggttttctcagtggcagagaatgacattccctacgagattgacttggacaatcttggcacactgtgtagctgggtcgtcgatggtcagtggaacatgccattcacagcacaccccaaggtagccccagggtcaggagagttagtcatatatgggtttaacattgtgaagcccttcctaactattggagttgtctcagaggatggaaagaaacttgaacggaaggttgatctcaagctagaaagatgtacatattgccatgaaattggggtcactaaaatgtacaatattatcatggatatgcctcttacagtggaccttactaggattctaagaggagctccgttgattgattttgaaacagaaagttatgcaagaattggagtcatgccccgtcatggagatgcagattcagtaatatggttcgatgtagaaccattttgcacattgcatctcatcaattgctttgaggaagatcatgaggttgtcatcagaggatttcgagtgccgggttccataattactggcataacacttgagcacacggctaatgaagagccagctaatcaaggacccagtgagaaatcttttcctcgcttatatgagtggagattaaacatgaaaagcagggctgtcacagtgatcaataacaaatatgctggcctgcatcacaagtatgcatatgcacaagtgattgatgtccaagggagcctggaaggtggctgtggaacagtgcgaccaaaatttggaggtttcgcaaaactacatcttcaagataacaacaaggcacatgtcattgatgctcagagatttgaaaatggaccaattgctaagataacattgccacaaagagtaccctatggctttcatggcacattcatcccaagaactacatacaagaagacatga

SEQ ID NO: 42 - protein, 478 aa, Oryza sativa
MVDGVVTLLEARKLVGKVILAGSANLYDGSIHEQSNFAPVDEIGGRTEIWRIEGTISDDFPEGVYIRNGSNPLFGALHKVNSIFGQSEDIWVEGEGMLHALYFTKSREGNTWSVSYNNRYVQSDTFNTERDRQRPCFLSAIKGDPLAIIAASILNMLRFGKVFRNMSNTGVFEHAERVFSVAENDIPYEIDLDNLGTLCSWVVDGQWNMPFTAHPKVAPGSGELVIYGFNIVKPFLTIGVVSEDGKKLERKVDLKLERCTYCHEIGVTKMYNIIMDMPLTVDLTRILRGAPLIDFETESYARIGVMPRHGDADSVIWFDVEPFCTLHLINCFEEDHEVVIRGFRVPGSIITGITLEHTANEEPANQGPSEKSFPRLYEWRLNMKSRAVTVINNKYAGLHHKYAYAQVIDVQGSLEGGCGTVRPKFGGFAKLHLQDNNKAHVIDAQRFENGPIAKITLPQRVPYGFHGTFIPRTTYKKT

SEQ ID NO: 43 - DNA, 1647 bp, Oryza sativa
atgatgacagcttctctgcatccatgtgtctgcaaggcctctcctgccttcagacctgcctcttctctgggtgcaagaacccagccaaaatccacagccacaaacccaaagagacctttgtttcaggagcttcagaggcgactttctttcaggattgacgaagcctcaaaggcgctggagacagcaaaacaggggcttttggatgcgttggtcgactcaaccttcaagttttctgaccaacctatgctcccatcagagaacaattttgccccagtcaatgagatcagtgaagccatagagattctgcagattgaaggagagatacctgaagatttcccagagggttccaatccactgtttggagccctccattcgaccgtctcaatcttcggaaaatccagcgaaatatgggttgaaggcgagggcatgctccatgccatctacttcacaaagaacagttcagatacttggtcagtctcctatgcaaatcgatatgtgcaatctgaaacattaaagattgagaaaaccagacagaagccatgtttcctccctgccatcatgggcgactctgcagccatcgtcgcggcctacattctcaactatatgaggtttggcaaagtgaacaagaacatcagcaacaccaatgtatttgagcatgctggaaaggtgtatgctgtttctgagaaccacctgcctcaggaaatcagcattcagaatctcgacacgggtgatagctgggacattaatggggaatggaaaaggcctttcacagctcacccaaaggttgctcctggatcaggagagctagtcatttttggttcagatgcaaagaggcctttcctaatggtcggagttgtctcagctgatggaactcaactaaaacataaagttgacctcaaactggacagatgtatactctgccatgacataggagttactgttaagtacaacataataatggacatacctcttaccatcgacatcagtagacttatcagaggcaatcagttgatcaagtttgagaaggacagctatgcaagaataggagttatgccccgttatggtgatgcagaatcagttatgtggtttgacgttgaaccattctgcatgttccattttatcaactgctttgaagagggtgatgaggttgttatcaggggctttcgtgcggctgactccatcattccaggccctcggatcagcctaaacaaaaatgatttgctctctgatccttctaagtgttctgtgaaacaaggaattaatgaagaatttttctctcgattataccagtggagattaaacacgaagacaaaggctgtttcagggcaatatttaagtggaactgagttttccatggaattccctgtgatcaatgaccactacacaggcttgcatcatagttatgcctatgcacaagtggtggattctttggaaagctcttacggcgttaatgagaaagtaatcctgaaatatggaggccttgcaaaactttgtcttgaagaggcagataatgtaattgcagaggtgcacattattgacgcccaaacatttgaaggtgctcctgtggccaaaatagtattgccacaaagagtaccctatggttttcatggaacgttcagatcatcactagcaaacacaatgacgtga

SEQ ID NO: 44 - protein, 548 aa, Oryza sativa
MMTASLHPCVCKASPAFRPASSLGARTQPKSTATNPKRPLFQELQRRLSFRIDEASKALETAKQGLLDALVDSTFKFSDQPMLPSENNFAPVNEISEAIEILQIEGEIPEDFPEGSNPLFGALHSTVSIFGKSSEIWVEGEGMLHAIYFTKNSSDTWSVSYANRYVQSETLKIEKTRQKPCFLPAIMGDSAAIVAAYILNYMRFGKVNKNISNTNVFEHAGKVYAVSENHLPQEISIQNLDTGDSWDINGEWKRPFTAHPKVAPGSGELVIFGSDAKRPFLMVGVVSADGTQLKHKVDLKLDRCILCHDIGVTVKYNIIMDIPLTIDISRLIRGNQLIKFEKDSYARIGVMPRYGDAESVMWFDVEPFCMFHFINCFEEGDEVVIRGFRAADSIIPGPRISLNKNDLLSDPSKCSVKQGINEEFFSRLYQWRLNTKTKAVSGQYLSGTEFSMEFPVINDHYTGLHHSYAYAQVVDSLESSYGVNEKVILKYGGLAKLCLEEADNVIAEVHIIDAQTFEGAPVAKIVLPQRVPYGFHGTFRSSLANTMT

SEQ ID NO: 45 - DNA, 1710 bp, Oryza sativa
atgtctcccgctatgctgcaggcgtcgtcgctgtgcgtatccgcggcgctgtcaggcgccgcgagccggccgggccgcctggccagccaggggcaccagggcaagcgggccgtggcgcagcctctcgcggctagcgccgtgacggaggcagcgccgcccgcgccggtcgtcgcgccgccggcccgccccgtcgacgccccgcggcgccgtggcggacgtggcggcggcggaggcggcggcgagctcgtggcgtggaagagtgtacggcaggagaggtgggagggtgcgctcgaggtggacggagagctgcctctctggctggatggcacgtacctgaggaacggcccgggactatggaacctcggcgactacggcttccggcacctgttcgacggctacgcgacgctggtgcgcgtctcgttccgcggcggccgcgccgtgggcgcgcaccggcagatcgagtcggaggcgtacaaggcggcgcgcgcgcacggcaaggtgtgctaccgcgagttctcggaggtgcccaagccggacaacttcctgtcctacgtcggccagctggcgaccctcttctcgggctcgtcgctcaccgacaactccaacaccggcgtcgtcatgctcggcgacggccgcgtgctctgcctcacggagaccatcaagggctccatccaggtcgacccggacacgctcgacacggtcggcaagttccagtacacggacaagctgggcgggctgatccactcggcgcacccgatcgtgaccgacaccgagttctggacgctgatccccgacctgatccggcccggctacgtggtggcgaggatggacgccggtagcaacgagaggcagttcgtcggcagggtggactgccgcggcgggccggcgccagggtgggtgcactcgttccccgtcaccgagcactacgtcgtcgtgccggagatgccgctccgctactgcgccaagaacctcctccgcgccgagcccacgccgctgtacaagttcgagtggcacctcgagtccggcagctacatgcacgtcatgtgcaaggccagcggcaagattgtggcgagcgtggaggtgccgccgttcgtgacgttccacttcatcaacgcgtacgaggagacggacgaggaggggcgcgtgacggcgatcatcgccgactgctgcgagcacaacgccaacaccgccatcctcgacaagctccgcctccacaacctccgctcctccagcggccaggacgtcctccccgacgccagggtggggcggttcaggatccccctggacgggagccagttcggcgagctggagacggcgctggacccggaggagcacgggcggggcatggacatgtgcagcatcaacccggcgcacgtcggcagggagtaccggtacgcctacgcctgcggcgcccgccggccgtgcaacttccccaacacgctcaccaaggtcgacctggtggagaggacggccaagaactggcacgaggagggctccgtgccgtccgagcccttcttcgtgccacgccccggcgccaccgaggaagacgacggcgtggcgatatcgatggtgagcgccaaggacgggtcgggctatgcgctggtgctggacggcaagacgttcgaggaggtcgcgcgggccaagttcccgtacgggctgccctacggcttgcactgctgctgggtgcccaggaaaaggaacagcaagtaa

SEQ ID NO: 46 - protein, 569 aa, Oryza sativa
MSPAMLQASSLCVSAALSGAASRPGRLASQGHQGKRAVAQPLAASAVTEAAPPAPVVAPPARPVDAPRRRGGRGGGGGGGELVAWKSVRQERWEGALEVDGELPLWLDGTYLRNGPGLWNLGDYGFRHLFDGYATLVRVSFRGGRAVGAHRQIESEAYKAARAHGKVCYREFSEVPKPDNFLSYVGQLATLFSGSSLTDNSNTGVVMLGDGRVLCLTETIKGSIQVDPDTLDTVGKFQYTDKLGGLIHSAHPIVTDTEFWTLIPDLIRPGYVVARMDAGSNERQFVGRVDCRGGPAPGWVHSFPVTEHYVVVPEMPLRYCAKNLLRAEPTPLYKFEWHLESGSYMHVMCKASGKIVASVEVPPFVTFHFINAYEETDEEGRVTAIIADCCEHNANTAILDKLRLHNLRSSSGQDVLPDARVGRFRIPLDGSQFGELETALDPEEHGRGMDMCSINPAHVGREYRYAYACGARRPCNFPNTLTKVDLVERTAKNWHEEGSVPSEPFFVPRPGATEEDDGVAISMVSAKDGSGYALVLDGKTFEEVARAKFPYGLPYGLHCCWVPRKRNSK

SEQ ID NO: 47 - DNA, 1830 bp, Oryza sativa
atggcaacacaagcgattgcaccgatgcacgccgccgtcgtgcaccgccaccacgttctaccaccccgccgctgcgtgcgccgccgtggcgtcttcgtccgcgcctcggccgccgccgccgccgccgccgccgagacggacacgctgtccgcggccttctgggactacaacctcctcttccggtcgcagcgcgacgagtgcctcgactccatcccgctccgcgtcaccgagggcgcgatcccgcccgacttcccggccggcacctactacctcgccgggccgggcatcttctccgacgaccacggctccaccgtccaccccctcgacggccacggctacctccgctccttccgcttccggcccggcgaccgcaccatccactactccgcgcggttcgtggagacggcggcgaagagggaggagagccgggacggcgcgtcgtggcggttcacgcaccgggggcccttctccgtgctgcagggcgggaagaaggtgggcaatgtgaaggtgatgaagaacgtggccaacaccagcgtgctgcggtggggcggccggctgctctgcctctgggagggcggccagccgtacgaggttgacccccggacgctcgagaccgtcggcccgttcgacctgctcggcctcgccgccgccgacgacaacaaggcaacgaacgcgtctgcagcacgacggccgtggctgcaggaggccggcctcgacgccgccgcgcgcctgctgcgccctgttcttagcggggtgttcgacatgccgggcaagaggctgctggcgcactacaagatcgacccgcggcgggggcgtctgctgatggtcgcctgcaacgccgaggacatgctcctcccgcgatcccacttcactttctacgagttcgacgcccacttcgacctcgtccagaagcgtgagttcgtcgtgccggaccacctcatgatccacgactgggccttcaccgacacccactacatcctcctcggcaacaggatcaagctcgacatccccggatcgctgctggcattgacgggcactcacccgatgatcgcggcgctggccgtggacccgagaaggcagtcgacgccggtgtacctgcttccgcgctccccggagaccgaggcgggcggccgcgactggagcgtgccgatcgaggcgccgtcgcagatgtggtccgtgcacgtcggcaacgcgttcgaggaggcgaaccgccggggcggcctcgacgtccggctgcacatgtcaagctgctcctaccagtggttccatttccacaggatgtttggttacaattggcaccacaagaagctggacccgtcgttcatgaacgcggcgaagggaaaggagtggctgcctcgcctcgttcaggtggccatcgagctcgacaggacgggagagtgccggaggtgctcagtcaggaggctgtccgatcagcacgccaggccggcggacttcccggcgataaacccaagctacgccaaccagaggaaccggttcgtctacgccggcgccgcgtccggctcccgcagattcctcccgtacttcccgttcgacagcgtggtgaaggtcgacgtctccgatggatcggcgcggtggtggtctaccgacgggcgcaagttcgtcggcgagccggtcttcgtcccgaccggcggcggagaggatggtggctatgttcttcttgtagagtatgcagtctccaagcacagatgccatctagtggtgctggatgcaaagaagatagggacagagaatgcacttgtggcaaaactagaggtgccaaagaacctcacttttccaatgggattccatggtttctggggagatgaatga

SEQ ID NO: 48 - protein, 609 aa, Oryza sativa
MATQAIAPMHAAVVHRHHVLPPRRCVRRRGVFVRASAAAAAAAAETDTLSAAFWDYNLLFRSQRDECLDSIPLRVTEGAIPPDFPAGTYYLAGPGIFSDDHGSTVHPLDGHGYLRSFRFRPGDRTIHYSARFVETAAKREESRDGASWRFTHRGPFSVLQGGKKVGNVKVMKNVANTSVLRWGGRLLCLWEGGQPYEVDPRTLETVGPFDLLGLAAADDNKATNASAARRPWLQEAGLDAAARLLRPVLSGVFDMPGKRLLAHYKIDPRRGRLLMVACNAEDMLLPRSHFTFYEFDAHFDLVQKREFVVPDHLMIHDWAFTDTHYILLGNRIKLDIPGSLLALTGTHPMIAALAVDPRRQSTPVYLLPRSPETEAGGRDWSVPIEAPSQMWSVHVGNAFEEANRRGGLDVRLHMSSCSYQWFHFHRMFGYNWHHKKLDPSFMNAAKGKEWLPRLVQVAIELDRTGECRRCSVRRLSDQHARPADFPAINPSYANQRNRFVYAGAASGSRRFLPYFPFDSVVKVDVSDGSARWWSTDGRKFVGEPVFVPTGGGEDGGYVLLVEYAVSKHRCHLVVLDAKKIGTENALVAKLEVPKNLTFPMGFHGFWGDE

SEQ ID NO: 49 - DNA, 1644 bp, Oryza sativa
atgggaggcggcgatggcgatgaggtgctgctgctgccggagccgcgccctcgcaggggcctcgcctcctgggcgctcgatctgctggagcgcgccgccgtccgcctcggccacgacgcctccaagccgctctactggctctccggcaacttcgcccccgtccaccacgagaccccgccggccccggccctccccgtccgcggccacctccccgagtgcttgaatggagaatttgtcagggtaggacccaatccaaagtttgtccctgtcgctggctatcattggtttgatggagatggaatgatccatgcgatgcgtatcaaagatgggaaagctacctatgtgtcgagatatgtgaagacttctcgtctcaagcaagaagagtattttggtggagcaaagtttatgaagattggagacctgaagggattttatggattgtttatggtccaaatgcaacaactccggaaaaaactcaaagtattggattttacatatggacatgggacagctaatactgcacttatctatcaccatggtaaacttatggccctgtcagaggcagataagccctgtaagttctcatcccataatgatgttgttaaggtccttgaagatggagacttgcaaactcttggattgttggattacgacaaacggttgaaacactctttcactgctcatccaaaggtcgatccatttacagacgaaatgttcgcttttggatattcgcatgaacctccttactgtacataccgggtcattaccaaggatggagccatgcttgatcctgtgccaataacaattccagaatctgtaatgatgcacgactttgccattacagagaattattctattttcatggaccttcctctgttgttccgaccaaaggaaatggtgaagaatggtgagtttatctacaagtttgatcctacaaagaaagctcgttttggtatactccaacgttatgaaaaggatgacacaaacatcagatggtttgaacttcccaactgcttcatattccacaatgctaatgcttgggaagagggtgacgaagttatcctaattacctgccgcttggagaatcctgacttggacaaggtgaacggttaccaaagcgacaatctcgagaactttgggaatgagctgtatgagatgagattcaacatgaaaaccggtgctgcttcacaaaagcaactatctgtttctgctgtagattttcctcgaattaatgagagctacactggcagaaagcagcggtatgtgtattgcgctattctaaacagcatagcgaaggtagcaggcattataaaatttgatctacatgctgaaccggagatcagtggctttaaacaacttgaagtggggggaaatgtgagaggaatatttgacttgggacctggtagattcgggtccgaggcaatttttgtgcctagggaacccggtgtatctggagaagaagatgatggttatttgatattctttgtccacgacgagaatacagggaaatctgaagtcaatgtgattgatgcaaagacaatgtctgctgatccggtggcagttgttgagctaccaagccgagttccctacggattccatgctttctttataaacgaggaacaactggcaaaacaatcagcgtga

SEQ ID NO: 50 - protein, 547 aa, Oryza sativa
MGGGDGDEVLLLPEPRPRRGLASWALDLLERAAVRLGHDASKPLYWLSGNFAPVHHETPPAPALPVRGHLPECLNGEFVRVGPNPKFVPVAGYHWFDGDGMIHAMRIKDGKATYVSRYVKTSRLKQEEYFGGAKFMKIGDLKGFYGLFMVQMQQLRKKLKVLDFTYGHGTANTALIYHHGKLMALSEADKPCKFSSHNDVVKVLEDGDLQTLGLLDYDKRLKHSFTAHPKVDPFTDEMFAFGYSHEPPYCTYRVITKDGAMLDPVPITIPESVMMHDFAITENYSIFMDLPLLFRPKEMVKNGEFIYKFDPTKKARFGILQRYEKDDTNIRWFELPNCFIFHNANAWEEGDEVILITCRLENPDLDKVNGYQSDNLENFGNELYEMRFNMKTGAASQKQLSVSAVDFPRINESYTGRKQRYVYCAILNSIAKVAGIIKFDLHAEPEISGFKQLEVGGNVRGIFDLGPGRFGSEAIFVPREPGVSGEEDDGYLIFFVHDENTGKSEVNVIDAKTMSADPVAVVELPSRVPYGFHAFFINEEQLAKQSA